(19) United States
(12) Patent Application Publication
(10) Pub. No.: US 2005/0282263 A1
(43) Pub. Date: Dec. 22, 2005

(54) Flexible vaccine assembly and vaccine delivery platform

(75) Inventors: Alison A. McCormick, Vacaville, CA (US); Mark L. Smith, Davis, CA (US); Kenneth E. Palmer, Vacaville, CA (US); John A. Lindbo, Wooster, OH (US); Long V. Nguyen, Vacaville, CA (US); Gregory P. Pogue, Vacaville, CA (US)

Correspondence Address: LARGE SCALE BIOLOGY CORPORATION, 3333 VACA VALLEY PARKWAY, SUITE 1000, VACAVILLE, CA, 95688, US

(73) Assignee: LARGE SCALE BIOLOGY CORPORATION, Vacaville, CA (US)

(21) Appl. No.: 11/090,497

(22) Filed: Mar. 25, 2005

Related U.S. Application Data
(63) Continuation-in-part of application No. 10/654,200, filed on Sep. 3, 2003.
(63) Continuation-in-part of application No. 10/457,082, filed on Jun. 6, 2003.
(60) Provisional application No. 60/556,931, filed on Mar. 25, 2004.

(51) Int. Cl. C12N 7/00; C12N 15/86

(52) U.S. Cl. 435/235.1; 435/456

(57) ABSTRACT

Herein described are various methods for making a vaccine that are made of re-assembled virus like particles (VLP). First, the VLPs are disassembled into coat proteins or encapsidation intermediate populations. Each population undergoes, for instance, chemical conjugation of unique peptide or nucleic moieties to form separate populations. Thereafter, a predetermined amount of each of the several (one or more) different coat proteins or encapsidation intermediates from the different populations is mixed and joined, forming intact VLPs, surrounding a nucleic acid core, that are composed of different coat proteins such that the reassembled VLP displays more than one peptide or other molecule. The nucleic acid can function either as a scaffold alone or can be engineered for the expression of an immunomodulatory protein in a eukaryotic cell.

### PRIORITY CLAIM

This application is a continuation-in-part of U.S. Provisional Patent Application No. 60/556,931, filed Mar. 25, 2004, and U.S. patent application Ser. No. 10/654,200, filed Sep. 3, 2003, U.S. patent application Ser. No. 10/457,082, filed Jun. 6, 2003, and U.S. Provisional Application No. 60/386,921, filed Jun. 7, 2002, all of which are incorporated herein by reference in their entirety.

This invention was made with United States Government Support under cooperative agreement number 70NANB2H3048 awarded by the National Institute of Standards and Technology.

### FIELD OF THE INVENTION

The invention relates to a novel vaccine platform that includes a reassembled virus constructed from one or more subunits, each subunit containing a different peptide or nucleic acid moiety added by genetic fusion or in vitro conjugation such that each subunit incorporates a target therapeutic agent. The invention further relates to a method for assembling RNA molecules in vitro for delivery and expression in eukaryotic cells. In particular, the invention provides for proteins, molecules and nucleic acid sequences necessary for the packaging of RNA molecules for delivery and expression in a eukaryotic cell. The packaged RNA molecules of the invention are capable of delivery to a wide range of eukaryotic cells. The packaged RNA molecules may also be targeted to specific eukaryotic cells. The invention further includes a delivery platform where the above described reassembled viruses or virus-like particles (VLPs), RNA vaccines are used to induce either cellular or humoral immunity, or both simultaneously, by the synergistic action of peptide fusions to the virus or VLP structure and the encoded proteins of the RNA.

### BACKGROUND OF THE INVENTION

To date, most traditional vaccines have been composed of live-attenuated or inactivated whole pathogen preparations. Generation of these sorts of vaccines is limited by the requirement for long and intensive basic research and development. Reliable production and scale-up technologies for live-attenuated or inactivated vaccines would be almost impossible to develop at short notice. There is, therefore, a need for the development of a safe, robust and broadly-useful technology that is suitable for the production of vaccines against unanticipated infectious disease threats. Vaccines developed from plant-virus-pathogen chimera's may provide a method to rapidly produce vaccines that can be used to prevent or treat a number of known or emerging disease threats.

Controlling immune responses to pathogens and tumor cells has been the focus of immunology, cell biology and pharmaceutical development for several decades. Much has been learned about the complexity of immune cells and the patterns and effect of cytokine expression in response to pathogen challenge, and vaccine administration. One key aspect of this work has been the identification of two major arms of the immune response, the Th1 response, which is largely cellular, and the Th2 response, which is predominantly humoral. The two types of immune responses are mounted in response to how foreign antigens are presented to the immune system, what cytokines are expressed by presenting cells and what types of immune cells are activated. Th1 responses result in cytotoxic immune cell function and production of neutralizing antibodies of a different subtype than observed with Th2 responses. While some pathogens can be susceptible to Th2 responses, the Th1 response is key to mounting an effective response to both pathogen and tumor cells. However, both pathogens and tumor cells have developed strategies to avoid immune surveillance, bypassing mechanisms that are essential to Th1 immunity.

A key goal in vaccine development is to direct Th1 type immunity, in addition to Th2 humoral responses, upon vaccine administration to the host. By using an attenuated cowpox virus, Jenner unknowingly took advantage of the powerful activation of Th1 pathway to prevent smallpox infections. Since his time, most pathogen vaccines have been killed or attenuated, which have generally shown good success in controlling pathogen morbidity and viral spread. However, two aspects of recent vaccine development have led to growing concerns for live or attenuated viral vaccines. The use of an attenuated or killed virus to treat human immunodeficiency virus (HIV) is impractical for several reasons. Occupational safety concerns, low yield of attenuated virus, and the threat of viral mutation or escape are serious drawback to both vaccine development and public acceptance. In other cases, as observed with measles virus and respiratory syncytial virus (RSV), unpredictable and severe adverse events are associated with whole virus immunization. Therefore, much research has focused on “subunit” vaccines, which are composed of pathogen protein(s) or peptides that are generally targeted by the host immune response for protective immunity (Vaccines, 3^{rd} ed 1999, Plotkin and Orenstein, Philadelphia Pa., Saunders Co). Unfortunately, protein subunit vaccines don't often elicit strong Th1 responses by themselves, and DNA subunit vaccines often fail to elicit antibodies. In most cases both antibodies and CTL responses are necessary in controlling pathogenesis or disease progression.

Two new types of vaccines have been created to overcome the deficiencies of current subunit vaccines. Non-pathogenic viruses have been genetically modified to encode immunogenic subunit proteins of a pathogen, thus taking advantage of the Th1 immune response to viral antigen presentation. Strong Th1 type immune responses have been demonstrated for many pathogen and self-antigens using adenovirus, vaccinia, fowlpox and alphavirus delivery systems (Walther and Stein. 2000 Drugs 60, 249). However, these “first generation” viral delivery systems encountered problems due to the vector immunogenicity, which precluded their subsequent use in booster immunizations. Viral priming followed by either protein or DNA boosting has been successful, but this approach requires the manufacture of at least two agents for a single vaccine. The large-scale manufacture of DNA and/or protein for these vaccines has encountered both technical and financial challenges.

A second strategy takes advantage of the self-assembly of viral coat proteins into virus like particles (VLPs), which by themselves stimulate strong Th1 antigen responses (Schiller and Lowy. 2001 Expert Opin Biol Ther. 1, 571). VLPs constructed from arrayed viral coat have been shown to be effective in stimulating both neutralizing antibody and cytotoxic T lymphocyte (CTL) responses. Viral coat proteins are also effective carriers of antigens through fusion to the external solvent-exposed residues, usually by genetic fusion (Pogue et al. 2002 Ann Rev Phyto Path 40, 3; Da Silva. 1999 Curr Opin Mol Ther 1, 82). Though promising, VLP technology also has drawbacks. Production is again limiting, and often fusion of a heterologous antigen to the coat reduces VLP yield, solubility, or prevents self-assembly. In addition, immune clearance, the same mechanism that limits whole virus boosting, also limits the use of VLPs. Clearly, there is a need for a cost effective viral coat antigen delivery system that overcomes the limitations of both whole virus and VLP technology for vaccine delivery. The properties of this system would include all the benefits of boosting Th1 responses via a virus-like antigen presentation to the immune system without pathogenicity, flexibility to rotate the VLP backbone to which the antigen is fused, generation of and control of immunogenicity, high yield and low cost.

Applicant and others have shown that coat proteins from plant viruses have all the immunologic presentation properties of animal virus coat, but without pathogenicity. A large number of positive (+) strand RNA plant viruses, including Tobacco Mosaic Virus (TMV), type member of the tobamovirus family, have been cloned and manipulated in vitro to express heterologous gene products in plants as well as to display biologically relevant peptides on its virion surface. A unique property of TMV virions is their ability to be disassociated to form monomers and self assemble into VLPs using a RNA scaffold. Plant coat proteins, including TMV, engineered to display foreign epitopes have been shown to promote functional immunity to both self-antigens (Savelyeva N 2001 Nat Biotechnol 19 760) and various pathogens (Pogue et al. 2002 Ann Rev Phyto Path 40, 3).

Essential for the encapsidation of the viral genomic RNA molecule into an infectious particle is the presence of a sequence element referred to as the origin of assembly (OAS). The TMV OAS is located approximately 1 Kb from the 3′ end of the viral genome and consists of a 440 nucleotide sequence that is predicted to form three hairpin stem-loop structures (Turner and Butler, 1986). The viral coat protein disks initially bind to loop 1 during viral assembly. In vitro packaging assays using mutual assembly origin transcripts have defined the 75 nucleotides comprising loop 1 as necessary and sufficient for encapsidation of foreign or viral RNA sequences (Turner et al., 1988). In vitro reconstitution studies have shown that preparations of purified coat protein, derived from virions from infected plant cells, are able to assemble into helical structures with TMV RNA at pH 7.0, resulting in assembly of TMV-like viral particles containing RNA (Fraenkel-Conrat and Williams, 1955). Furthermore, it has been shown that foreign chimeric RNA molecules containing OAS sequences, transcribed in vitro using SP6 or T7 RNA polymerase, may be assembled in vitro into pseudovirus particles (Sleat et al., 1986).

The cloning and sequencing of the viral coat proteins responsible for encapsidation has led to the insertion of these genes into bacterial expression vectors in, for example, E. coli (Shire et al., 1990). However, in vitro assembly with recombinant E. coli viral coat proteins results in a decreased reconstitution rate relative to native coat protein produced in plants (Shire et al., 1990). U.S. Pat. No. 5,443,969 attempts to overcome this deficiency in E. coli by packaging RNA sequences containing a TMV-OAS in vivo in E. coli, instead of in vitro. However, introduction of the encapsidated viral vectors into hosts outside of plants is problematic. The lack of acetylation of the TMV coat protein in E. coli results in poorly efficient encapsidation of non-capped RNAs. These RNAs are poorly translated in eukaryotic cells due to the lack of the cap structure. Further, the yields of recombinant TMV products in E. coli are very poor and not commercially feasible.

The process of intracellular delivery of genetic material for therapeutic purposes by either correcting an existing abnormality or providing cells with a new function is the basis behind gene therapy (Drew and Martin, 1999), and for DNA immunizations. Practically speaking, nucleic acid immunization technologies present an attractive front-line defense against new pathogens: there is probably no other system that can compete as the first line in a rapid-response subunit vaccine strategy. However, conventional DNA vaccines suffer from a number of significant drawbacks that makes reliance on this technology alone unwise. Most significantly, the dose of DNA required to stimulate an effective immune responses is very high, with the implication that production of significant quantities for large scale immunization will be challenging. DNA and RNA vaccines are generally capable of promoting good Th1 type cytotoxic T cell responses, which are essential for elimination of non-cytopathic pathogens. However, with few exceptions, the antibody response induced by DNA vaccines is poor. Hence, although nucleic acid vaccines are attractive from the prospective that production can be very rapid, ideally an initial DNA or RNA vaccination should be followed by a booster vaccination, preferably with protein, to induce efficient antibody production and more complete protection against pathogen challenge. The current invention addresses the issues raised above by introducing a novel and flexible vaccine delivery platform

### SUMMARY OF THE INVENTION

The present invention includes several unique solutions that address current limitations of VLP technology, while retaining all the positive characteristics of a successful VLP antigen scaffold. Applicant presents a method for generating VLP vaccines in adaptable, predictable, stable and scaleable manners. This work is highly innovative, and there is continuing development. The method includes generating muli-valent vaccines where different vaccine protein moieties are fused to the surface of a single VLP structure conferring a multi- functional effect—the availability of immune peptides (protein elements stimulating protective immunity) and peptides that either modulate the host immune response or facilitate efficient immune cell recognition or processing. The proposed vaccines will be also bi-functional, where the protein elements of the VLP, with or without a peptide fusion or series of fusions, encapsidate a modified RNA moiety. The modified RNA can carry an mRNA of interest and that protected RNA can then be used to carry nucleic acid content, along with protein, into an immune cell that takes up the vaccine. The RNA constituents works synergistically to generate strong, lasting immunological responses by encoding either an intact pathogen or oncology antigen, proteins that stimulate host immune responses or proteins that modulate either a type Th1 or Th2 immune response to the vaccine. The method alleviates problems associated with other VLP systems by having robust production potential, improved cellular uptake, and multi- epitope valency. A selection of structurally similar, yet immunologically distinct VLP carriers allows rotation of the coat backbone for prime-boost strategies that have proven unworkable in other VLP systems.

Vaccination with bi-functional RNAs presents an alternative to DNA vaccination, with some distinct advantages. In the first instance, there is little concern that an RNA-based vaccine could cause oncogenesis because it cannot incorporate into or transform the genome. Secondly, there is good evidence that one could deliver an RNA vaccine derived from an RNA virus (such as an alphavirus) as a safe self-amplifying vaccine vector. Alphavirus replicons are cytolytic for cells, and thus the replicating RNA vaccine is intrinsically transient and self- eliminating. Alphavirus “replicon” vaccines cause powerful immune responses-both antibody and cell-mediated-associated with both increases in the amount of antigen produced as well as the production of inflammatory cytokines induced by intracellular accumulation of the viral dsRNA replicative intermediate. These features indicate that the dosage of replicative RNA required for induction of effective immune responses would be orders of magnitude lower than that required by DNA immunization. However, the major drawback associated with naked RNA vaccines is the notoriously labile nature of the nucleic acid: this severely limits the application of RNA vaccines for mass immunizations.

Alphavirus replicon vaccines are currently delivered either as naked RNA transcribed in vitro, packaged in alphavirus-like particles (replicon particles), or as plasmids containing infectious cDNAs, driven by the cytomegalovirus immediate early promoter (CMV promoter). Replicon particles are very efficient as vehicles for carrying the replicon RNAs into cells, but production is complicated, inefficient and unreliable. An efficient packaging and RNA stabilization technology is therefore required to protect alphavirus-based RNA vaccines from degradation. Two viable options present themselves: (1) to deliver recombinant alphavirus constructs as infectious cDNA plasmids; (2) to package alphavirus RNA transcribed in vitro such that it is protected from nucleases and has good stability and storage properties. An approach for the latter option is presented below.

The inventors employ as a VLP carrier the well-characterized plant virus, tobacco mosaic virus (TMV), and exploit its unique abilities to reconstitute VLP structures in vitro onto various heterologous RNA sequences.

By introducing a cysteine in the solvent exposed sequences of TMV coat, we can introduce and fuse foreign antigen epitopes ex-vivo. Epitope sequences that are not amenable to in vitro synthesis will be fused in-frame genetically to the TMV coat protein. TMV VLPs will be reassembled in vitro decorated with a single epitope (monovalent), or with a collection of different epitopes (multivalent), derived from in vitro conjugation or expressed from a genetic fusion. Other easily modified amino acids or series of amino acids constituting a recognizable site (e.g. a glycosylation site) may also be employed as a target for ex-vitro attachment of an epitope sequence. These are particularly advantageous when the epitope is a polysaccharide, non-amino acid hapten, a sequence too large for genetic fusion, a combination of these, etc.

As a scaffold for reassembly, the present invention includes using an RNA that encodes a protein that will enhance vaccine potency, thereby creating a bi-functional antigen delivery system that derives its activity from both protein and nucleic acid. The RNA can also incorporate an alphavirus replicon to augment translation. Essential for the encapsidation of the RNA molecule by the TMV coat protein, to generate an RNA-containing VLP, is the presence of the 75 nucleotide sequence comprising loop 1 of the origin of assembly (OAS). By combining this 75 nucleotide sequence with foreign sequences encoding protein(s) or peptide(s) of therapeutic interest, the RNA molecule can function as an effective scaffold for the generation of a TMV-like VLP. The RNA can encode any number of immunomodulating factors (e.g. IL4, IL1β or IFNγ) that ensure a highly successful immune response to the vaccine, and help generate either protective or therapeutic immunity to the pathogen, or deliver inhibitory RNA signal (RNAi) for targeted gene inhibition. This VLP strategy can be applied to effectively target immune cells and stimulate Th1 type responses.

An important requirement to inducing a Th1 type immune response is getting VLPs into cells for processing and antigen presentation. Peptides with known cell targeting have been identified (Samuel O., Shai, Y., 2001 Bichem. 40, 1340; Magnusson et al. 2001 J. Virol. 75 7280; Bushkin-Harav et al. 1998 FEBS L. 424 243) and can be tested in vitro by direct examination of cell entry, and in vivo for augmented antigen presentation by examining the type and speed of immune response to target antigens. Targeting and fusion peptides will be tested for their ability to augment cellular uptake of TMV, as well as their ability to deliver encapsidated RNA in vitro and in vivo.

A common method to improving vaccination is to co-administer an adjuvant or a specific T-helper peptide to stimulate T-cell help. CpG DNA has been shown to be an easily administered adjuvant that improves Th1 type immune responses when co-administered with an appropriate vaccine (Krieg. 2000 Vaccine 19, 618). Most CpG DNA adjuvants have been given mixed with the vaccine and administered subcutaneously (s.c.), although the single strand thiolated DNA can also be fused to a protein carrier through SPDP conjugation chemistry. Also, several universal T-helper peptides have been identified (Kulkarni, A. B., et al., 1995 J. Virol. 69,1261; Panina-Bordignon, 1989 Eu. J. Imm. 19, 2237; Boraschi, 1988 J Exp Med. 168,675; Weiner, G. et al., 1997 Proc. Nat. Acad. Sci 94 10833). Immunostimulatory peptides, usually fragments of cytokines, have also been identified that direct Th1 type immunity after vaccination in combination with pathogen or self-antigen peptides or subunit vaccines (IL1, Boraschi, 1988 J Exp Med. 168,675). Coat fusions containing T-helper or adjuvant peptides or CpG DNA oligo will be used to augment the immunogenicity of co-expressed peptides, or encapsidated RNA. Many different adjuvants have been used previously, some of which are general in nature and others used to enhance certain types of responses. These adjuvants are known per se and may be used in the present invention.

Lastly, it is well established that cytokines play an important role in determining which arm of the immune system is activated after vaccine delivery. Interleukin 4 (IL4) has been implicated in directing Th2 type immune responses and interferon gamma (IFNY) is an important contributor to Th1 responses (Spellberg and Edwards. 2001 Clin Infect Dis 32, 76). By introducing IL4 and IFNγ RNA into cells by encapsidation into a TMV VLP, we may be able to influence the type of immune response that is generated. Applicant can test both antibody isotype responses to antigen, which are a reflection of Th1 or Th2 antigen presentation, as well as assess CTL responses that are primarily a consequence of Th1 immunity.

Cell fusion peptides, T-help, adjuvants, pathogen antigens, tumor antigens and encapsidated cytokine RNA will be tested systematically in combination with antigens from Papillomavirus and melanoma murine disease models. Immunogencity and challenge models will establish incremental improvements over vaccination with single peptides, and define the best peptide/RNA combinations for generating Th1 or Th2 immune responses.

The availability of such a flexible and effective vaccine platform provides opportunities to apply non-live vaccines for humans and livestock thus reducing side effects and increasing effectiveness. New vistas of medical practice, including applications for breaking self-tolerance and driving immune responses against weak antigens, may be opened by the synergistic and high specific-activity of the disclosed vaccine platform.

The invention relates to a method where a specified virus, such as a tobacco mosaic virus (TMV), is disrupted into a plurality of subunits. Each subunit contains a genetically fused peptide or is subjected to a conjugation reaction in order to attach a predetermined epitope, peptide or nucleotide thereto. A plurality of subunits are processed in this manner to produce a plurality of subunit groups, where one subunit group has attached thereto a predetermined peptide; another subunit group has a second peptide; another subunit has a predetermined epitope attached there to; and another subunit group has a nucleotide attached thereto, and so on, for as many subunit groups necessary to provide the building blocks for a plurality of virus vaccines.

An alternative strategy is to employ TMV RNA modified to initiate internal ribosomal entry by introducing specific sequences known to cause such an effect. These internal ribosomal entry sites (IRES) are effective in causing internal translation products from a polycystronic RNA in animal cells (Yang et al., J Virol 1989 63(4):1651-60). Introduction of an IRES into a TMV genome in frame with an RNA encoding either a full length gene product or immunostimulatory cytokine or other kind of immunmodulatory protein allows for translation of that protein. Because these IRES are introduced into non-replicating RNA, the amount of TMV and proportional transcript taken up by a cell after vaccination is conceivably lower than with a self replicating RNA such as encoded by an alphavirus replicon, but the level of translation product should be sufficient to induce the correct response.

The present invention includes research and development of technological solutions to help the USA to produce and supply effective vaccine reagents in response to unanticipated pathogen threats. Applicant specifically addresses issues that limit bio-defense application of nucleic acid vaccines: poor environmental stability and high dosage requirements. In addressing these issues, we will draw upon the core of knowledge that the inventors possesses in the field of positive stranded RNA viruses and their applications in biotechnology to develop a set of molecular tools to improve nucleic acid vaccines. Applicant will also demonstrate our capacity to produce protein subunit vaccines that will provide effective antibody responses. Production of protein subunit vaccines is inherently slower than nucleic acid vaccines and so, practically, will only be available within a delayed period following encounter with a new pathogen threat. However, the inventor's non-transgenic plant-based vaccine expression platform (GENEWARE®) has the capability to express a variety of proteins, including virus-like particles (VLP)—known to be potent inducers of antibodies in vaccinated individuals—rapidly. Applicant has recently used a modified TMV expression vector to produce 16 different human therapeutic vaccines in tobacco plants, and have shown excellent safety in a Phase I clinical trial (BB-IND #9283). Unlike other competing technologies, GENEWARE® does not require specialized fermentation facilities, and uses the efficient, rapid protein production strategy of the plant virus TMV to harness plant protein production machinery to produce vaccine proteins. A typical harvest time, post inoculation is less than 21 days. Since the same virus is used from pilot testing to large-scale manufacturing, there is little or no transition time between validation and manufacturing scale up. Most of the delay in delivery of vaccines via GENEWARE® technology would be in the growth of plants, and establishment of antigen-specific purification protocols. These aspects of the technology result in a low cost of production for plant-derived VLP vaccines.

### LIST OF FIGURES

FIG. 1 is a flow diagram outlining the standard methods for the generation of multivalent vaccines via chemical fusions

FIG. 2 is a flow diagram outlining methods to generate multivalent TMV-based vaccines via chemical fusions that can be bifunctional through the use of a translatable RNA species as a scaffold

FIG. 3 is a flow diagram outlining the standard methods for the generation of multivalent vaccines via genetic fusions

FIG. 4 is a flow diagram outlining methods to generate multivalent TMV-based vaccines via genetic fusions that can be bifunctional through the use of a translatable RNA species as a scaffold

FIG. 5 is a rendering of TMV virion disassembly and in vitro virion reassembly, showing from left to right: an electron micrograph of a single TMV virion; space filling models of an individual TMV coat protein, with schematic placement of surface exposed N—(N) and C—(C) terminal domains and surface exposed loop (SL); space filling models of 20S disk subunits; and a reassembled VLP surrounding RNA.

FIG. 6 is a schematic of in vitro conjugation, or molecular fusion, of heterologous peptides of various biological functionalities to modified TMV 20S subunits and reassembly of heteropolymeric (multiple peptide display) VLP surrounding bioactive RNA.

FIG. 7 shows the expression levels of TMV-HA peptide fusions at different insertion sites in TMV U1 coat protein. N. Benthamiana plants (21 days post sow) were inoculated with encapsidated RNA with a mild abrasive and approximately 200 μg tissue was harvested 9 to 10 days post infection. Samples were ground in 300 μl acetate buffer pH 5, and insoluble material was pelleted by centrifugation. Total plant proteins were harvested by grinding 100 μg tissue in 100 μl SDS-PAGE buffer. The soluble supernatant was removed and then the pellet was resuspended in 200 μl Tris buffer pH 7.5 for a final pH extraction at pH7. 10 μl of each sample was then separated by 10-20% SDS-PAGE, stained in Coomassie brilliant blue and destained before photographing. HA N accumulates as a pH5 insoluble pH7 soluble coat fusion at approximately 19 kD (arrow). HA Loop is expressed, but insoluble (present in total SDS grind but not soluble fractions 5 or 7). HA GPAT is expressed and soluble at pH5 but is cleaved, and only partially cleaved at pH7. Ha C is expressed and is insoluble at pH5 and soluble at pH7 with minor cleavage products visible. 5: Acetate buffer pH5; 7: Tris buffer pH 7; S: SDS PAGE buffer total tissue grind.

FIG. 8 shows TMV proteins that were harvested from plants infected with p15eTMV or p15e DE TMV after signs of infection were evident. 20 mg leaf discs were then processed in Acetate buffer A: 50 mM Na-acetate (pH 5.0)/5 mM EDTA, then the insoluble material was resuspended in tris buffer T: 50 mM TRIS (pH 7.5)/10 mM EDTA, and material was compared to processing in SDS page buffer S: 78 mM TRIS (7.0)/10% (w/v) sodium dodecyl sulfate/0.05% bromophenyl blue/6.25% Glycerol/10% β-mercaptoethanol, for total protein analysis. Materials were then separated by SDS-PAGE, and visualized by Coomassie staining. The control was U1: wild type coat protein of tobacco mosaic virus strain U1, M: protein molecular weight standard.

FIG. 9A (1) shows the nucleic acid and amino acid composition for N terminal Cysteine TMV U1 (Seq ID No: 19). Alternatively the cysteine can be incorporated into other tobamovirus coats and at other positions within the coat protein, e.g., 60 s loop, C terminus, read through position. (2) Composition for N terminal Lysine TMV U1 (Seq ID No: 20). Alternatively the lysine can be incorporated into other tobamovirus coats and at other positions within the coat protein, e.g., 60 s loop, C terminus, read through position.

FIG. 9B shows chemical conjugation to cysteine containing TMV coat protein by glutaraldehyde. 1.0 mg of peptide was mixed with 1.0 mg of Cyst-N TMV (C—N), in a volume of 1 ml, and a 20 μl sample was removed for T=0. This sample was added to 20 μl of water and 40 μl of 2× PAGE buffer, and immediately boiled. (The water in T=0 equalizes its concentration with that of T=4 which has added glutaraldehyde.) Glutaraldehyde was added to the reaction to a final concentration of 1%, in a final volume of 2 ml. The reaction was allowed to proceed for 4 hours at room temperature, with constant rotation. After 4 hours, a sample was removed for a T=4 time point, added to an equal volume of 2× PAGE buffer and immediately boiled. 8 μl (2 μg peptide & 2 μg carrier) of each time point was loaded on a gel for Western transfer to nitrocellulose, and 16 μl (4 μg peptide & 4 μg carrier) of each time point was loaded on a gel for Coomassie staining.

FIG. 10 shows transmission electron micrograph (TEM) images of TMV wild-type and myc or V5 N terminal fusion virus. TMV, TMV-myc-N or TMV-V5-N were coated onto 400-mesh carbon-coated copper grids at 20 to 80 μg/ml. Samples were then negatively stained with 1% phosphotungstic acid, dried and stored at RT until visualized using a Philips CM120 TEM, at 37,000× magnification. The bar represents 130 nm.

FIG. 11 (A) shows a flow diagram for the purification of TMV U1 virus from infected plant material. (B) SDS-PAGE analysis (10-20% tris-glycine gel) for the isolation of TMV U1 from infected N tabacum MD609 plants. Since the majority of the virus partitioned into the S1 supernatant the S2 supernatant was not processed. GJ, green juice; S1, supernatant S1; S1 PEG 1, resuspended virus from the first PEG precipitation; S1 PEG2, resuspended virus from the second PEG precipitation.

FIG. 12 shows an SDS gel (10-20% tris glycine) illustrating the effect of salt on the virus partitioning between the S1 and S2 process streams for the Cysteine N coat protein fusion. GJ, initial green juice; S1, S1 process stream; S2, S2 process stream.

FIG. 13 shows a flow diagram for the precipitation of TMV virus in the presence of polyethylene glycol (PEG) and sodium chloride (NaCl).

FIG. 14 shows a flow diagram for the generation of free coat protein from TMV virus.

FIG. 15 (A) shows the ultraviolet absorption spectrum for TMV U1 coat protein at pH 8.0. (B to D) Treatment of Myc N coat protein with DEAE Sepharose to remove contaminating residual RNA. (B) and (C) Comparison of the ultraviolet absorbance spectrum before and after DEAE resin treatment. (D) Agarose gel electrophoresis to track contaminating RNA. Following binding of the starting coat (L) to the DEAE resin, the coat protein was eluted with 50 mM NaCl (E50) yielding a preparation free from RNA. 500 mM NaCl was required to elute the RNA from the resin (E500). FT represents the resin flow through.

FIG. 16 shows the change in the chromatogram for TMV U1 coat protein, analyzed by size exclusion chromatography, before and after incubation at room temperature. (A) Chromatogram profile for coat protein stored at 4° C. (B) Chromatogram profile for coat protein following storage for 16 hours at room temperature. A YMC-Pack Diol-300 column (5 μm bead; pore size, 30 nm) was employed and the flow rate was 0.5 ml/min. The buffer employed was 0.1 M phosphate, pH 7.0 at either 4° C. or room temperature, based on the temperature of the sample injected.

FIG. 17 (A) Shows the kinetics of virion reconstitution from viral RNA plus the U1 coat protein, which were followed by the increase in solution turbidity at 310 nm. This is approximately proportional to the average rod length. TMV virus, at a molarity equivalent to that of the starting RNA, was employed to indicate the optical density of a fully reconstituted in vitro encapsidation. (1) Standard IVE conditions; 0.1 M sodium phosphate, pH 7.2. (2) 0.1 M phosphate pH 7.2 with RNasin at 0.4 U/μl. (3) 0.1 M sodium pyrophosphate, pH 7.2. (B) Agarose gel electrophoresis of final reassembly reactions to assess RNA integrity. RNA Cntrl, RNA lacking coat protein; PO₄, phosphate buffered reassembly reaction; Pyro PO4, pyrophosphate buffered reassembly reaction; PO₄ RNasin, phosphate buffered reassembly reaction containing RNasin.

FIG. 18. (A) shows the A310 nm kinetic profile for reassembly reactions (IVE) with the ELDKWAS coat protein fusion, in the presence and absence of RNasin. The ELDKWAS virus control was present at the same molar concentration as the RNA in the reassembly reactions. (B) Agarose gel electrophoresis of reassembly reactions 5 hours after initiation. Reassembly reactions were performed in the presence (+) or absence (−) of RNasin and the coat protein employed is indicated. RNA alone, at the same concentration as in the reassembly reactions, was run as a control.

FIG. 19 shows images and data analysis for reassembly reactions viewed by transmission electron microscopy (TEM). The samples were negatively stained with 1% phosphotungstic acid, dried and stored at RT until visualized using a Philips CM120 TEM, at 37,000× magnification. (A) Coat protein control sample (no RNA present) (B) reassembly reaction with the same coat protein concentration as in (A) but with TMV RNA present at 50 μg/ml. Image is for reassembly reaction performed in the presence of RNasin. The scale bar represents 200 nm. (C) Comparison of the normalized particle size distribution for reassembly reactions with the ELDKWAS coat protein fusion, performed in the presence and absence of RNasin. n indicates the number of rods counted in the electron microscopy images.

FIG. 20 (A) shows the A310 nm kinetic profile for separate reassembly reactions (IVE) with the ELDKWAS, Myc and HPV ep2 coat protein fusions, all performed in the presence of RNasin. Wild type TMV RNA was employed as a scaffold. The ELDKWAS virus control was present at the same molar concentration as the RNA in the reassembly reactions. The RNA alone control is also shown, however, the coat protein alone and HPV ep2 and Myc virus controls are omitted for clarity. (B) shows the A310 nm kinetic profile for bivalent reassembly reactions (IVE) with the ELDKWAS, Myc and HPV ep2 coat protein fusions taken in pair wise combinations. All reassembly reactions were performed in the presence of RNasin and wild type TMV RNA was employed as a scaffold. The ELDKWAS virus control was present at the same molar concentration as the RNA in the reassembly reactions. The RNA alone control is also shown, however, the coat protein alone and HPV ep2 and Myc virus controls are omitted for clarity.

FIG. 21 shows MALDI and SDS-PAGE data for the CRPV 2.1 coat protein fusion. (A) MALDI TOF trace showing the spectrum for purified CRPV 2.1 coat protein fusion. The predicted sequence weight for the protein, with the Met cleaved is 19320 Da, in excellent agreement with the observed molecular weight. (B) SDS-PAGE gel for the CRPV 2.1 coat protein fusion, showing a protein purity of greater than 97% for the final virus preparation.

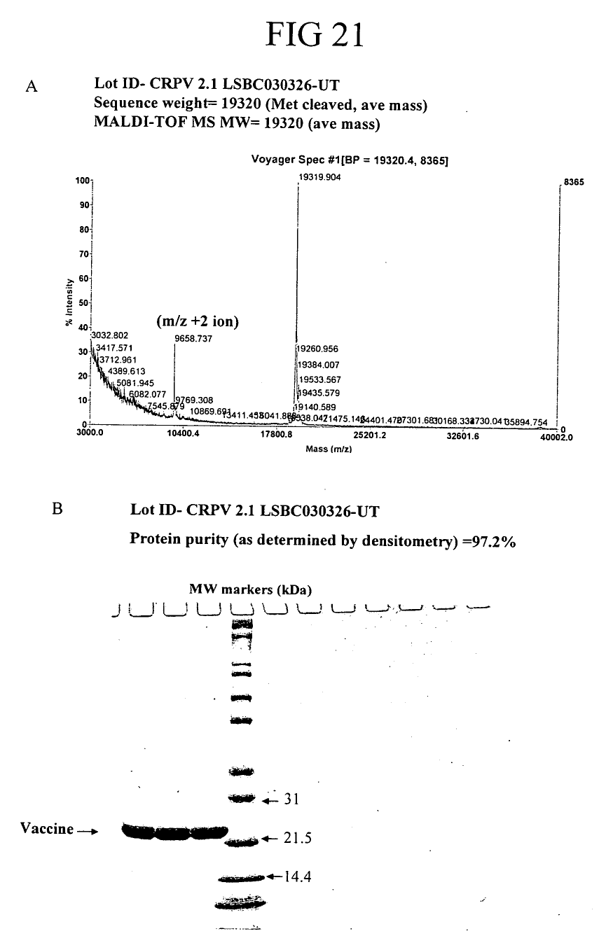

FIG. 22 shows various RNA constructs which may be used as a scaffold for the reassembly of multivalent TMV-based vaccines and which impart bifunctionality to the reconstituted virion, by virtue of the gene (s) that they encode. (A) TMV RNA containing a structural or non-structural gene. (B) TMV RNA containing IRES structural or non-structural gene. (C) Alphavirus replicon containing TMV OAS and structural or non-structural gene. (D) Chimeric mRNA containing TMV OAS and Omega with structural, non-structural or immune modulatory gene. (E) Chimeric mRNA containing TMV OAS with structural, non-structural or immune modulatory gene. For illustrative purposes the Figure shows the CRPV L1 or CRPV E7 genes in the RNA constructs. However, melanoma associated gene e.g. p15e, GP100 or any other structural or non-structural gene can replace these CRPV-associated genes.

FIG. 23 shows humoral responses to TMV coat fusion vaccines as measured by ELISA against the peptide. Sera were collected 10 days post vaccine 3 (pV3), serially diluted onto ELISA plates coated with either a c-myc-BSA conjugate or a foreign antigen (FNR) V5 fusion. Plates were then reacted with anti-mouse HRP and positives were visualized using a colorimetric substrate, and quantitated using statistical software. Commercially available positive controls were used as standards.

FIG. 24 shows the cellular response to TMV ova G vaccination as measured by fluorescent assisted cell sorting (FACS). Spleens from animals vaccinated with TMV ova G were harvested, and then cultured with 1 μg/ml ova peptide for 5 hours in the presence of Brefeldin A. Cells were then fixed, incubated with anti-CD4-FITC or anti-CD8-FITC antibodies, and then refixed and permeabilized. Cells were then incubated with anti-IFN-PE or anti-TNF-PE, and then visualized for PE and FITC staining by flow cytometry.

FIG. 25 is a chart depicting attachment of epitopes fused to the coat protein of TMV by molecular fusion or chemical fusion. Epitopes are fused to the coat protein of TMV or to the intact virus by disulfide or amide bond, e.g. TMV-cys and Sulfo-LC-SPDP. This enhances solubility and yield.

FIG. 26 shows the various steps of the GENEWARE® process wherein plants are inoculated with the TMV virus having GFP and the TMV-GFP expression in tobacco plants.

FIG. 27 includes a gel image and MALDI mass spec data qualification data demonstrating purity of expression of papillomavirus CRPV 2.1 using GENEWARE®.

FIG. 28 is a chart showing the results of epitope-TMV fusions with rabbit papillomavirus L2 epitope produced in planta, extracted, purified and qualified, and tested in mice.

FIG. 29 is a chart showing a further embodiment of the present invention, wherein two different epitopes are fused to the coat protein of TMV, one epitope at the N positions and one at the C positions.

FIG. 30 is a chart showing comparisons between single epitope fusion vaccines vs dual epitope vaccines.

FIG. 31 is a chart showing results of test conducted in mice using a single epitope vaccine having p15e melanoma epitopes.

FIG. 32 is a chart showing results of c57B6 and T-cell activation.

FIG. 33 is a chart showing the results of B16 melanoma tumor experiments where p15e and a variant of p15e having DE amino acids attached thereto were tested on mice.

FIG. 34 is a chart showing the results of immune responses after immunization with various Ova preparations.

FIG. 35 is a chart showing the death points after challenge with Ova EG7 tumor cells.

FIG. 36 is a chart showing the results of IFNg responses to Ova SH vaccines with various adjuvants.

FIG. 37 is a chart showing the results of IFNg responses to various Ova antigen preparations.

FIG. 38 is a chart showing the death points after challenge with Ova EG7 tumor cells.

FIG. 39 is a chart showing the expression of a gene intracellularly.

FIG. 40 is a chart showing the titers of antibody against bGal after vaccination.

FIG. 41 is a chart showing the results of IFNg responses to various vaccines with peptide or protein stimulation.

FIG. 42 is a chart showing the antibody titers induced by various groups of vaccinated animals

FIG. 43 is a chart showing the results of IFNg responses after vaccination and stimulation.

### DETAILED DESCRIPTION OF THE INVENTION

Definitions and Abbreviations

In order to facilitate understanding of the invention, certain terms used throughout are herein defined:

“GM-CSF” means Granulocyte-Macrophage Colony Stimulating Factor. GM-CSF may increase the immunogenicity of antigens by stimulating antibody production mechanisms.

“Non-native” means not derived or obtained from the same species.

“Native” means derived or obtained from the same species.

“IgG” means immunoglobulin-G.

“Intergenic sequences” means the non-coding DNA sequences, wherein the viral origin of replication is situated, that are located between open reading frames of viruses.

“OAS” means origin of assembly sequence. The origin of assembly sequence is necessary for assembling the RNA molecule with viral coat proteins into a viral particle.

“Reconstituted protein” means the isolated and hydrated form of protein from a complex protein mixture

“IL4” means interleukin 4, a cytokine that activates immune cells, especially B cells

“IL1b” means Interleukin 1, beta subtype, a cytokine that activates immune cells

“IL1b peptide” means a 9 amino acid section of IL1b that can stimulate T cells

“IFNγ” means interferon, gamma subtype, a cytokine that activates immune cells, especially T cells

“TMV” means tobacco mosaic virus

“VLP” means virus like particle

“Th1” means T-helper type one immune response, which is characterized by both antibody and cellular immunity

“Th2” means T-helper type two immune response, which is characterized by primarily an antibody response

“IVE” means in vitro encapsidation

“RNA” means ribonucleic acid

“DNA” means deoxyribonucleic acid

“HA” means a peptide sequence derived from influenza hemaglutinin

“V5” means a peptide sequence derived from simian virus 5

“myc or Myc” means the peptide derived from the myc oncogene

“N” position means the position the peptide or modification is inserted, at the N terminal location of coat protein

“L” position means the position the peptide or modification is inserted, at the extracellular loop location of coat protein

“G or GPAT” means the position the peptide or modification is inserted, at four amino acids from the C terminal location of coat protein

“C” position means the position the peptide or modification is inserted, at the C terminal location of coat protein

“Cys” means the amino acid Cysteine

“20S” subunit describes the sedimentation profile of the 34 subunit coat protein disk in a density gradient

“4S” subunit describes the sedimentation profile of the 4 subunit coat in a density gradient, which is an intermediate to the formation of a 20S disk
“kDa” means kiloDalton, which refers to the molecular weight or mass of the protein
“TEM” means transmission electron microscopy
“RT” means room temperature
“4C” means 4 degrees Celsius, or near zero Fahrenheit
“PAGE” means polyacrylamide agarose gel electrophoresis
“SDS” means sodium dodecyl sulfate, a detergent
“PEG” means poly ethylene glycol (molecular weight 6000-8000) “NaCl” means sodium chloride, or salt
“DEAE” mean diethyl aminoethyl, a molecule used on anion exchange resins
“PO4” means phosphate
“pyro PO₄” means pyrophosphate
“SU” mean subunits
“CRPV” means cottontail rabbit papillomavirus
“ROPV” means rabbit oral papillomavirus
“HPV” means human papillomavirus
“OVA” means ovalbumin
“GJ” means green juice, or total plant homogenate
“S1” means clarified plant extract supernatant
“S2 means supernatant derived from the S1 insoluble material by resuspension at pH 7
“BSA” means bovine serum albumin
“MW MALDI” means molecular weight mass determination by Matrix Assisted Laser Desorption Ionisation mass spectrometry
w/v” means weight per volume
“OD” means optical density
“DDT” means Dithiothreitol
“RNAse” is an ubiquitous cellular enzyme that degrades RNA
“RNAsin” is a commercially available RNase inhibitor
“DEPC” is diethyl pyro carbonate, a chemical inhibitor of RNAse activity
“Nab” means neutralizing antibody
“L1” means papillomavirus capsid protein L1
“L2” means papillomavirus capsid protein L2
“E1,2,4,6,7, and E8” are papillomavirus early gene products
“CTL” means cytotoxic T lymphocyte
“SFV” means semliki forest virus
“IRES” means internal ribosomal entry site, which allows for the initiation of translation in the middle (or anywhere that is not at the first ATG) of the RNA
“ORF” means open reading frame, the functional unit of RNA, which when translated encodes a protein
“B16” means the mouse melanoma tumor cell line named B16
“SPDP” N-succinimidyl-3-(2-pyridyldithio) propionate
“BCA assay” Protein assay based on bicinchoninic acid

The present invention relates to a novel method for for the colorimetric detection and quantitation of total protein.

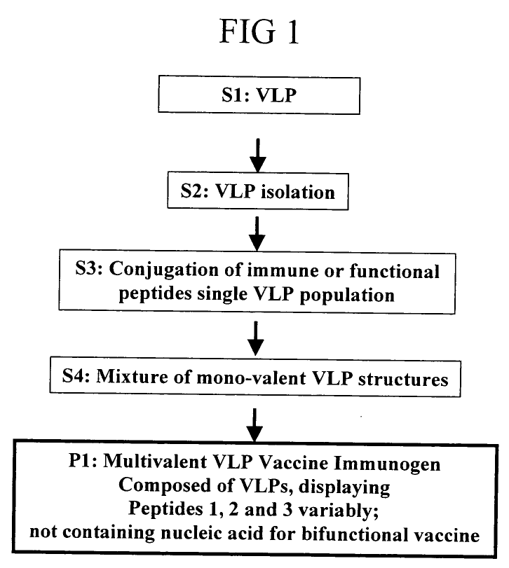

The present invention relates to a novel method for construction of a plurality of vaccines and pharmaceuticals using viruses, such as the tobacco mosaic virus (TMV). In broad terms, the invention is practiced in a manner depicted generically in FIGS. 1-6, as described below.

The description of the present invention is first provided in general terms, followed by a more detailed description that includes many bio-chemical procedures.

Standard methodologies can produce a pseudo-multivalent vaccine product by a chemical conjugation process outlined in FIG. 1. VLP particles are produced (S1) and isolated (S2). Individual peptides are individually chemically conjugated to the surface of independent lots of VLPs (S3) to produce distinct populations of VLPs, each displaying a unique peptide adduct. It is possible to conceive that multiple peptides could be simultaneously conjugated on the surface of the same population of VLPs to produce VLPs with a random distribution of unique peptides. The distinct populations of immune particles are then mixed (S4) to produce a population of VLP particles with distinct peptides covalently attached to the surface (P1). The resulting product will display distinct peptides in its mixture, but each will be independently taken up by immune cells and independently used to stimulate the immune system. There will be a lack of synergy between the fused peptides since there is no special connection between different peptides; each functions independently.

Mutivalent vaccines using the tobacco mosaic virus (TMV) coat protein involve the display of more than one peptide sequences on the same coat protein. Each peptide can be placed at one of at least the three surface exposed locations of the TMV coat protein (N, Loop, and C termini). These three positions are described above. Each peptide may be inserted at or attached to other locations as well. Since previous attempts at inserting various peptide epitopes has frequently resulted in problems with solubility and self assembly, it is particularly advantageous to have several different locations to try along with multiple different viral vectors. Such fusions can also be used to display more of a single peptide on the coat protein. At this time up to three different peptides may be displayed utilizing these three locations on the coat protein. In addition to the TMV coat protein, one may recombined between the coat protein of TMV (U1 strain) and tobacco mild green mosaic virus (TMGMV; U5 strain). This allows more flexibility in cases where a peptide may not be soluble in one carrier's but readily soluble in the second carrier's. The following table depicts different combinations of displays of one peptide on a single carrier and on recombined carriers in possession of the inventors. One can expand from this to several combination to display up to three different peptide sequences on the recombined carrier.

TABLE
Summary of the mutivalent peptide display on the TMV U1 coat
protein based on preliminary solubility results on SDS-PAGE

| Name of the | | C-terminal position |
|---|---|---|
| construct | N-terminal | (insertion at 4 amino |
| (sequence) | position | acids off the end; GPAT) |
| Int---Int | Integrin | Integrin (SGRGDSG) |
| | (SGRGDSG) | |
| Int---CRPV-L2.1 | Integrin | CRPV-L2.1 |
| | (SGRGDSG) | (VGPLDIVPEVADPGGPTL) |
| Int---ROPV-L2.2 | Integrin | ROPV-L2.2 |
| | (SGRGDSG) | (AGSSIVPLEEYPAEIPT) |
| Int---Ova | Integrin | Ova (SIINFEKL) |
| | (SGRGDSG) | |
| Int---IL1b | Integrin | IL1b (VQGEESNDK) |
| | (SGRGDSG) | |
| IL1b---IL1b | IL1b | IL1b (VQGEESNDK) |
| | (VQGEESNDK) | |
| IL1b---CRPV-L2.1 | IL1b | CRPV-L2.1 |
| | (VQGEESNDK) | (VGPLDIVPEVADPGGPTL) |
| IL1b---ROPV-L2.2 | IL1b | ROPV-L2.2 |
| | (VQGEESNDK) | (AGSSIVPLEEYPAEIPT) |
| IL1b---Ova | IL1b | Ova (SIINFEKL) |
| | (VQGEESNDK) | |
| IL1b---Int | IL1b | Integrin (SGRGDSG) |
| | (VQGEESNDK) | |

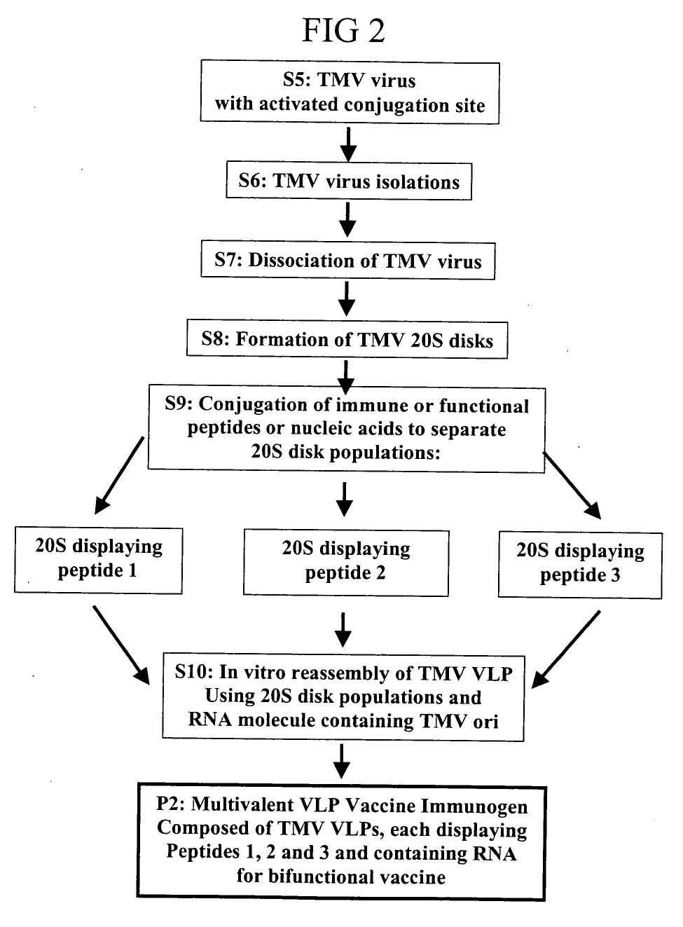

The described invention exploits the unique properties of the tobacco mosaic virus (TMV) that is amenable to the procedures outlined in FIG. 1, but also new methods (FIG. 2) with significant advantages. As represented by the box S5 in FIG. 1, a TMV virion is constructed with a surface associated amino acid allowing for improved chemical conjugation. This can be the presence of a unique, surface associated cysteine or lysine residue, although other methods can be employed. Large quantities of TMV are produced (S6) using, for instance, tobacco plants that are infected with the desired strain of TMV, then processed as described in co-pending patent application Ser. No. 09/962,527 filed Sep. 24, 2001, entitled PROCESS FOR ISOLATING AND PURIFYING VITAMINES AND SUGARS FROM PLANT SOURCES, and related U.S. Pat. Nos. 6,303,779, 6,033,895 and 6,037,456 all commonly assigned to Large Scale Biology Corporation, Vacaville, Calif., all of which are incorporated herein by reference in their entirety. Once large quantities of TMV are available, a process that is described in greater detail below disrupts the TMV in order to produce a large number of subunits (SU) or 20S disks, as represented by the box S7 and S8. The subunits are then separated at step S9 to form a plurality of subunits, each to be processed separately, as is described in greater detail below. As represented by step S9, each individual subunit group is subjected to a conjugation reaction in order to add predetermined components, such as a functional peptide, epitope, proteins or nucleic acid sequence to the subunits in that subunit group, in a manner that is described in greater detail below. As represented at step S10, pluralities of groups of subunits are now constructed into a single VLP structure where each subunit having specific epitopes, peptides, proteins or nucleotides attached thereto. TMV 20S disks naturally reassociate to form a rod-shaped virion surrounding an RNA molecule containing a unique sequence termed the TMV ori, or origin of assembly (OAS). This produces a multivalent vaccine (P2) that is not equivalent to a simple mixing reaction. Multifunctional peptide or nucleic acid adducts are linked physically to one another allowing each to synergistically enhance the cellular uptake of the VLP vaccine, immune processing, number of immune peptides presented to the immune system and the nature of the stimulated immune response. The simultaneous presentation of each peptide or nucleic acid component on the same VLP, rather than on distinct, unlinked VLP populations, is predicted to enhance the effectiveness of the VLP vaccine and lower the lower dose.

As an alternative to using disks, or other encapsulation intermediates for other viruses, one may use individual capsid proteins. Once these are modified by any of the methods of the present invention, they may then be used to self assemble into a virus or VLP.

Further basic steps in the method of the present invention are depicted in FIG. 2. Specifically, at step S10 a specific recombinant RNA sequence is selected to be the scaffold for assembly of the TMV VLPs. The specific VLP subunits selected in step S10 are combined with the RNA selected to form a reassembled TMV via a process that is described in greater detail below. The RNA can act only as a structural scaffold and could represent only the TMV RNA itself, not offering any augmented function other than a building block of the new VLP vaccine. However, recombinant RNAs can be constructed containing the TMV ori (S10) that also encode proteins. Once the VLP is taken up in immune cells, the TMV virion has unique function. It is preferentially bound by ribosomes and disassembled by a co-translational mechanism (Mundry et al., J Gen Virol. 1991 April;72 (Pt 4):769-77.). This would allow the efficient translation of this RNA so that the encoded protein is produced within the host immune cells. The encoded protein can either be an intact antigen to stimulate humoral or cellular immune responses against the targeted pathogen or cancer. Conversely, the RNA could encode immune stimulatory proteins (enhancing the amplitude of immune response) or modulatory proteins (insuring the direction, Th1 or Th2, of the immune response). This combination of protein elements that stimulate the immune response, as well as promoting the efficiency and effectiveness of the response,—in combination with an encoded nucleic acid component that is functional for augmenting the immune response, makes this vaccine truly bifunctional.

It should be noted that RNA is inherently unstable as a ‘naked’ element, or one not coated with a protective protein coating. However, it has an advantage over DNA in nucleic acid vaccines since it promotes translation of the desired product within immune cells, but is degraded and does not risk the immunized host with DNA recombination and the associated oncologic events. ‘Naked’ or uncoated nucleic acid vaccines of RNA or DNA types are very inefficient, where milligram (mg) quantities of DNA are required for any immune response in humans. Out of the mg of vaccine administered, picograms or less are taken up by immune cells. This results in expensive manufacturing and formulation costs, and very inefficient unpredictable immune responses. This invention allows the ‘naked’ RNA encoding important antigens or immune enhancing proteins to be coated and protected within the VLP structure of TMV. Such coating enhances the stability of the RNA and improves the delivery efficiency.

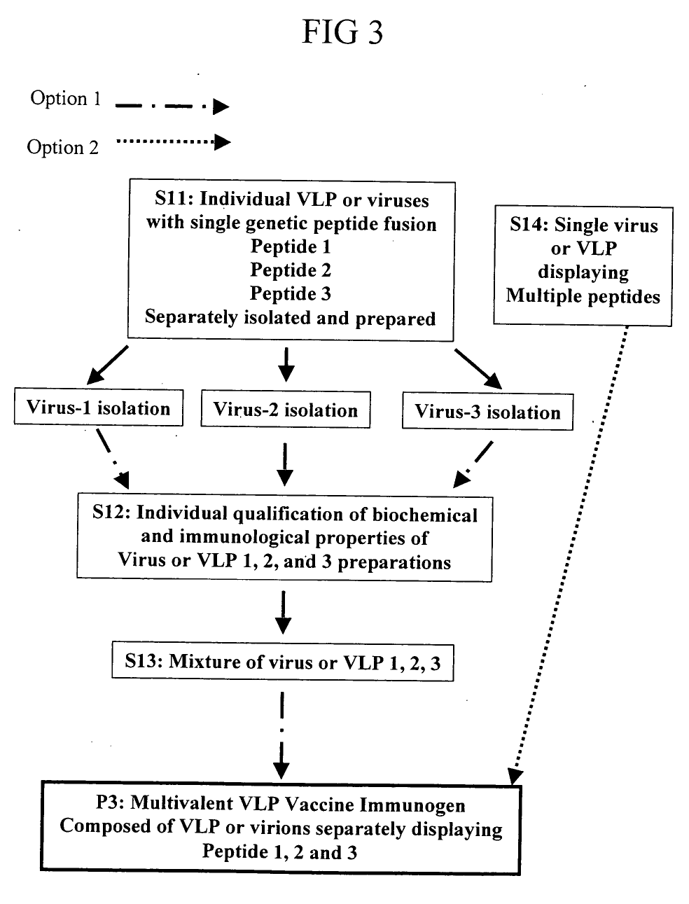

VLP vaccines are not dependent only on chemical conjugation to add immune peptides to their surface. The art describes methods for generating VLP vaccine through the genetic fusion of immunologically relevant peptides to the surface of VLPs. This process is described in FIG. 3. In this case, individual (S11) or multiple (S14) peptides are fused to the surface of the VLP protein through recombinant DNA procedures where the protein coding sequence for the immune peptide is fused to that of the VLP structure. Each individual or multi-peptide displayed VLP structure is purified (S11) and then qualified for its properties (S12). A multivalent vaccine is constructed by mixing either individual VLP populations displaying one or more peptides by genetic fusion (S13) or simply using a single population of VLP that is displaying more than one peptide by genetic means (S14). These procedures produce a multivalent VLP immunogen composed of multiple separate VLP populations, each displaying a unique immune peptide (P3). This approach suffers from the same limitations of the vaccines produced in FIG. 1 where little to no synergistic activity can be predicted by the simple mixture of non-linked peptides. Further, the VLP vaccines lack a nucleic acid component and are simply single functional vaccines—only providing a protein-based signal to the immune system.

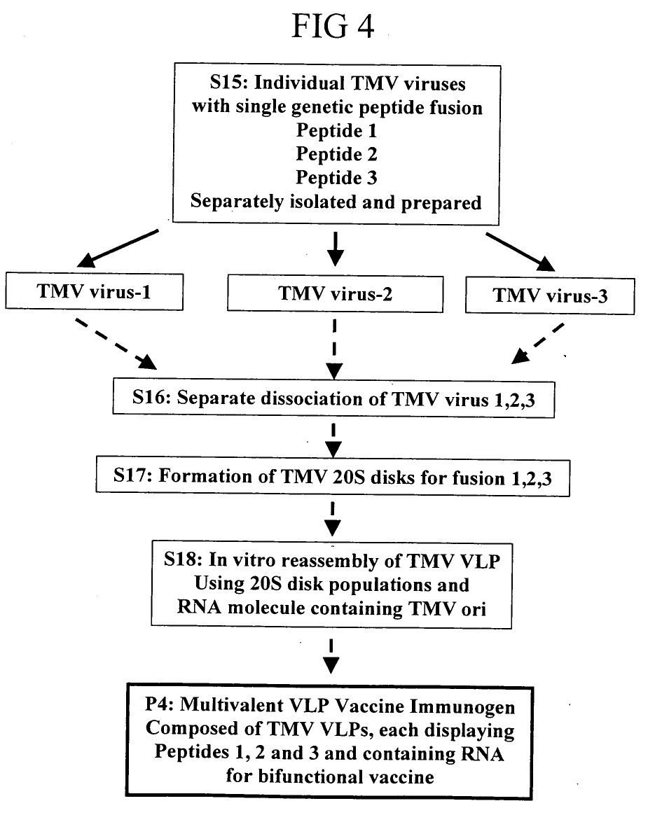

This invention overcomes these difficulties by allowing truly multi-valent and multi-functional vaccines to be derived. TMV is amenable to the same procedures described in FIG. 3 to produce mixtures of VLPs each with unique genetic fusions. However, its unique properties permit the procedure described in FIG. 4. Individual TMV virions can be prepared with single or multiple peptides by genetic means (S15). Each individual virion is isolated (S15) and qualified. Each TMV virion is separately disassembled (S16) and SU are prepared (S17) composed of 20S disks displaying a unique array of immune peptides. This plurality of SU are then reassembled surrounding a RNA containing the TMV ori to produce TMV VLP (S18). The final product is indeed a VLP vaccine that displays multiple immune peptides simultaneously on the surface of each VLP (P4) and contains RNA that functions both as a scaffold for VLP assembly and as a separate immune stimulus. The advantages of this approach are the same as described above in that the particle is multi-functional in terms of the plurality of immune, immune modulatory, immune stimulatory or cell uptake facilitating peptides simultaneously displayed on the surface of the VLP. This allows more efficient cellular uptake, processing and immune stimulation resulting in reduced dose and improved immune protection. The RNA again contributes essential functions beyond a scaffolding device. It can encode intact antigens, immune modulatory, immune stimulatory proteins to further augment the immune response. The RNA is protected within the VLP and is delivered efficiently to the cellular translation apparatus by the natural functions of TMV VLPs.

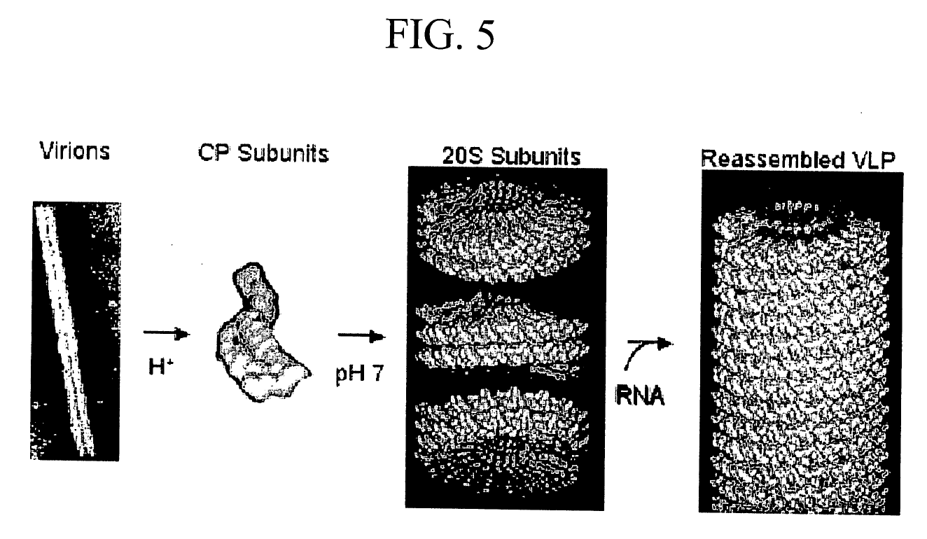

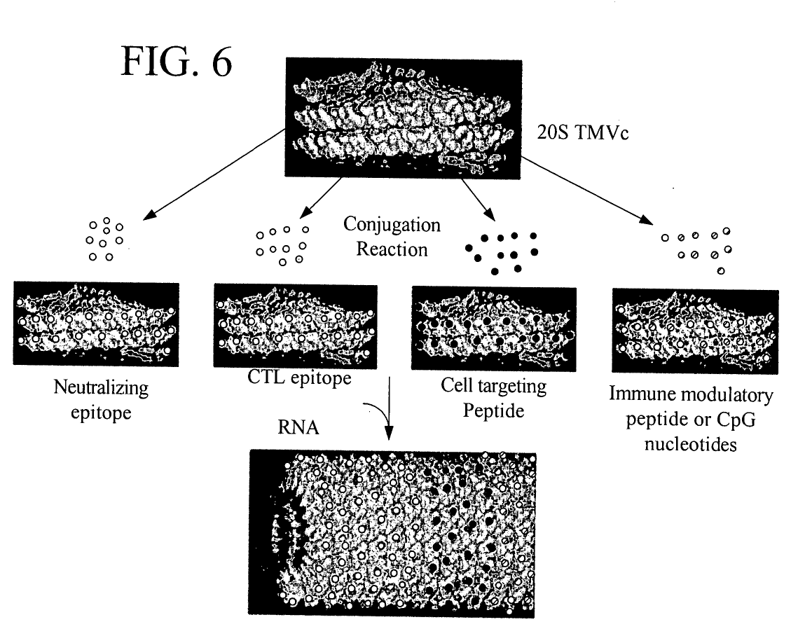

It should be understood that the above description is only a basic framework of steps upon which the present invention functions, and a basic understanding of the platform for constructing vaccines and pharmaceutical products in accordance with the present invention. The steps outlined in the flow diagrams in FIG. 2 and FIG. 4 are illustrated visually in FIGS. 5 and 6. Two further points should be noted with regard to the basic frameworks outlined in FIGS. 1 to 4. Firstly these figures indicate that various vaccine compositions contain 3 unique epitopes either displayed on separate VLPs or virions or all reassembled onto one VLP or virion. The number three was chosen purely for illustrative purposes and it should be understood that any number of epitopes can be recombined to form a multivalent vaccine. Secondly the entity displayed on the surface of the VLP or virion need not be limited to a peptide epitope as indicated in FIGS. 3 and 4. The displayed entity can also be a nucleotide, introduced by chemical fusion, or a complete protein, introduced by either chemical or genetic fusion. Furthermore all possible combinations of nucleotide, peptide epitope and complete protein, in terms of both number and ratio, can be envisioned for multivalent vaccine reassembly. For example peptide 1, nucleotide A and complete protein X, each displayed on separate virions or VLPs can be combined to yield a multivalent VLP vaccine similar to P3 in FIG. 3. Alternatively separate pools of 20S disks each displaying peptide 1, nucleotide A and complete protein X can be reassembled in vitro to generate a multivalent vaccine similar to P4 in FIG. 4, where all entities reside on a single VLP or virion.

The present invention may be used in many situations where one whishes an immune response to a number of different epitopes or antigens or microorganism/cell types from which they can be derived. For example there are many different strains of HPV and a vaccine against plural types is desirable, such as a bivalent vaccine with HPV L1 and L2 epitopes. Likewise for numerous other bacteria, fungi, parasites and viruses with constantly evolving antigens or for which numerous different strains exist such as influenza virus.

It may also be desirable to induce antibody production to multiple sites on an antigen in order to produce an antibody preparation well adapted for immunoassays, particularly “sandwich-type” binding assays. When one wishes to produce an antibody against a hapten(s) for assay purposes, the present invention is well suited as the generation of some anti-hapten antibodies with high specificity, affinity and avidity is problematic.

In the ideal situation, a multivalent vaccine could immunize an animal against many different pathogens with a single vaccine preparation. This would save time and costs, particularly for agricultural animals.

While the examples below are exemplified by using TMV vectors, it will be appreciated that other viruses with repeating capsid proteins may be used. Likewise, individual coat proteins or encapsulation intermediates of differing size from any virus may be used in the processes of the present invention.

While the examples below refer to in-vitro physical mixing of different viral coat proteins or encapsidation intermediates, the present invention may also mix in vivo during the normal synthesis of a virus.

In this embodiment, the virus may contain two or more different coat protein genes, each having a different epitope. As the virus replicates, it naturally produces both coat proteins and uses them randomly to package the viral nucleic acid. The resulting virus is a mosaic of the two or more different coat proteins. TMV has previously been used to produce several different non-TMV proteins by introducing the foreign gene into the TMV genome under the control of a subgenomic promotor. It would be preferred to include a second (or more) coat protein with a second epitope under control of the same subgenomic promotor as the first coat protein with a first epitope in order to produce approximately equal amounts of each coat protein, thereby assuring a suitable mosaic virus.

Production of Two or More Peptides on the Surface of TMV Particles In Vivo.

Here we describe an alternative method to display two or more peptides on the surface of tobamovirus particles in vivo. This method does not require re-assembly of the virus-like particle in vitro, and makes use of the GENEWARE® dual-subgenomic promoter technology as described in U.S. Pat. Nos. 5,316,931; 5,589,367; 5,866,785; 5,889,190 and others in the patent family entitled “Recombinant plant viral nucleic acids” and “Plant viral vectors having heterologous subgenomic promoters for systemic expression of foreign genes”.

GENEWARE® vector pGWHPV16L2.3 contains a recombinant tobacco mosaic virus (strain U5) coat protein that contains a human papillomavirus type 16 L2.3 peptide (sequence GTGGRTGYIPLGTRPPTATDT) fused near the C-terminus of the coat protein. We construct a similar recombinant U5 coat protein by polymerase chain reaction amplification of the wild type TMV U5 coat protein, with a fusion of a peptide encoding the human papillomavirus type 18 L2.3 peptide (sequence GTGSGTGGRTGYIPLGGRSNTVVDVG), with PacI (5′) and XhoI (3′) restriction sites flanking the recombinant U5 coat protein. This fragment is cloned as a PacI-XhoI fragment into the GENEWARE® vector pGWHPV16L2.3 to create dual subgenomic promoter vector pGWHPV16L2.3-HPV18L2.3. The DNA clone is transcribed in vitro according to methods established in the literature, and described in U.S. Pat. Nos. 5,316,931; 5,589,367; 5,866,785; 5,889,190 to generate infectious transcripts that are inoculated on Nicotiana benthamiana or other Nicotiana plants. Recombinant tobacco mosaic virus particles are isolated that comprise a virus particle chimera, with two different coat proteins, in this case TMV U5::HPV16L2.3 and TMVU5::HPV18L2.3. This process is illustrated in the attached FIGURE.

A preferred method to create these virus particles is to construct a synthetic gene for the second TMV coat protein gene, such that the sequence homology at the RNA level is as different as possible between the two coat proteins. This takes advantage of the degeneracy present in the genetic code to design a synthetic nucleotide sequence that is as different as possible to the native U5 gene sequence, but which still encodes the U5 coat protein.

The TMV coat protein gene used in this invention may be any one of the tobamovirus coat proteins. The second coat protein may or may not derive from the same tobamovirus species or strain, but it is anticipated that only closely related tobamovirus coat proteins will encapsidate the same RNA molecule.

Following are a series of detailed examples, which illustrate the general flow diagrams described on the preceding pages.

### EXAMPLE 1

### Peptide Fusions and Solubility as a Function of pH

The current industry standard for success with peptide fusions is 40-50%. To improve on this a series of fusions were tested at multiple insertion locations on the TMV U1 coat protein and each fusion was extracted under multiple conditions, to determine the influence of fusion position on virus solubility. This example describes the influence of genetic fusion position on the isolation of recombinant TMV viruses (step S15, FIG. 4).

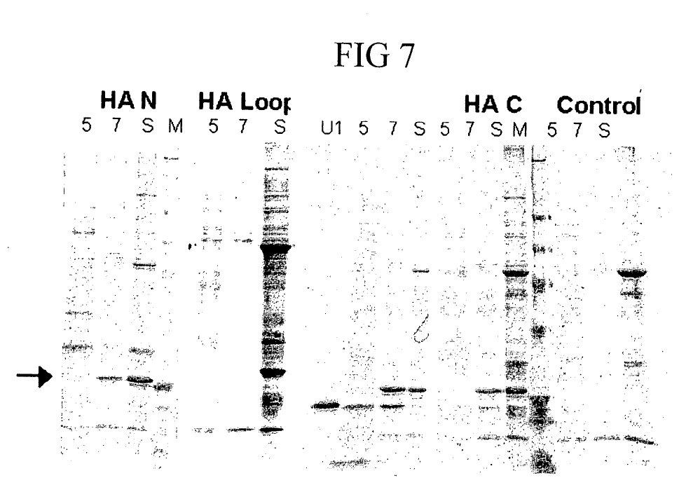

FIG. 7 illustrates results for the fusion HA, inserted at four different locations on the U1 coat protein; the N terminal, C terminal, surface loop (L) and 4 amino acids from the C terminus (GPAT). Clear differences in the extent of cleavage and virus solubility were evident. Approximately 100% HA GPAT was cleaved back to wild type U1 protein molecular weight when extracted at pH 5. Re-extraction at pH 7 improved full-length yield to 50%. Tissue extraction in SDS PAGE buffer yielded full-length coat fusion product, suggesting that cleavage was occurring during processing. This also occurred at the C terminal fusion location, although to a lesser extent. The processing of coat fusions appears to be site specific, as locating the epitope at the N-terminus yielded a full-length product. No virus was recovered at pH 5 or pH 7 with the HA epitope at the loop position; the SDS-PAGE buffer grind indicated that loop insertion was expressed but resulted in an insoluble product. For fusions that show cleavage during extraction e.g. HA GPAT, protease inhibitor cocktails can be incorporated to reduce or eliminate cleavage. Alternatively, other strains of N. tobaccum can be screened to identify hosts with reduced protease activity.

Table 1 summarizes the influence of epitope location on the solubility and relative recoveries for HA and two additional model epitope fusions, V5 and Myc. The V5, HA and myc epitope TMV fusion proteins were tested for reactivity to peptide specific antibodies by Western analysis, to confirm the identity and integrity of each fusion peptide (data not shown).

TABLE 1
Position of insert and Extraction buffer pH

| | N | | L | | GPAT | | C | |
|---|---|---|---|---|---|---|---|---|
| Fusion name | pH 5 | pH 7 | pH 5 | pH 7 | pH 5 | pH 7 | pH 5 | pH 7 |
| V5 | +++ | ++ | + | + | ++ | + | ++ | + |
| HA | − | ++ | − | − | − | ++ | − | ++ |
| Myc | +++ | ++ | − | +/− | ++ | +++ | ++ | ++ |

Table 1. Expression levels by insertion site for three antibody binding epitopes.

Following the confirmation of expression with the three model fusions the list of fusions was expanded to include clinically relevant epitopes of papillomavirus and melanoma as well as immuostimulatory and cell fusion epitopes aimed at incorporating biological functionality to reassembled fusion products. Table 2 summarizes the solubility results for all the epitope fusions. Of the 18 target epitopes attempted 15 were successfully expressed as soluble products, an 83% success rate. This represented a doubling of the previous industry standard of 40% expression/solubility. This improvement is due to the rotation of the insert position, performed in parallel with the extraction with two different pH buffers.

TABLE 2

| | | | |
|---|---|---|---|
| Peptides | Name | Solubility | Scalability |

| | | | | |
|---|---|---|---|---|
| GKPIPNPLLGLDSTK | (Seq ID No: 1) | V5 | N, G, C | N, G, C |
| YPYDVPDYAK | (Seq ID No: 2) | HA | N, G, C | G, C |
| EQKLISEEDLK | (Seq ID No: 3) | c-myc | N, G, C | N, G, C |
| Papillomavirus | | | | |
| VGPLDIVPEVADPGGPTLV | (Seq ID No: 4) | CRPV 2.1 | N, G | G |
| PGGPTLVSLHELPAETPY | (Seq ID No: 5) | CRPV 2.2 | N, G | G |
| VGPLEVIPEAVDPAGSSIV | (Seq ID No: 6) | ROPV 2.1 | N, G | G |
| PAGSSIVPLEEYPAEIPT | (Seq ID No: 7) | ROPV 2.2 | N, G | G |
| AALQAIELM | (Seq ID No: 8) | HPV16 ep2 | N | N |
| Melanoma | | | | |
| SVYDFFVWL | (Seq ID No: 9) | TRP-2181-188 | — | |
| KSPWFTTL | (Seq ID No: 10) | p15E 604-611 | — | |
| SIINFEKL | (Seq ID No: 11) | OVA | N, G, C | N, G |
| HIV | | | | |
| ELDKWAS | (Seq ID No: 12) | ELDKWAS | N | N |
| Immunostimulatory | | | | |
| CEYNVFHNKTFELPRA | (Seq ID No: 13) | Th SH 45-60 | G, C | |
| QYIKANSKFIGITELKK | (Seq ID No: 14) | P2 TT 830-846 | — | |
| VQGEESNDK | (Seq ID No: 15) | IL1β | N, G, C | N |
| Cell fusion | | | | |
| FAGVVLAGAALGVATAAQI | (Seq ID No: 16) | F1 Measles | L, G | |
| SGRGDSG | (Seq ID No: 17) | integrin | N, G, C | N |
| GYIGSR | (Seq ID No: 18) | laminin | N, G, C | N |

Table 2. 15 of 18 peptides have been expressed in frame with TMV U1 coat at either the N-terminus (N) the GPAT position (G) or at the C terminal location (C). Those fusions that were soluble in either pH 5 or 7 extraction buffer from leaf punch grinds (˜200 μg leaf tissue) are indicated in the Solubility column. Those fusions that were also successfully scaled up (>500 grams leaf tissue) are also indicated.

### EXAMPLE 2

### Improving Solubility and Accumulation by Modifying the Linker Amino Acids

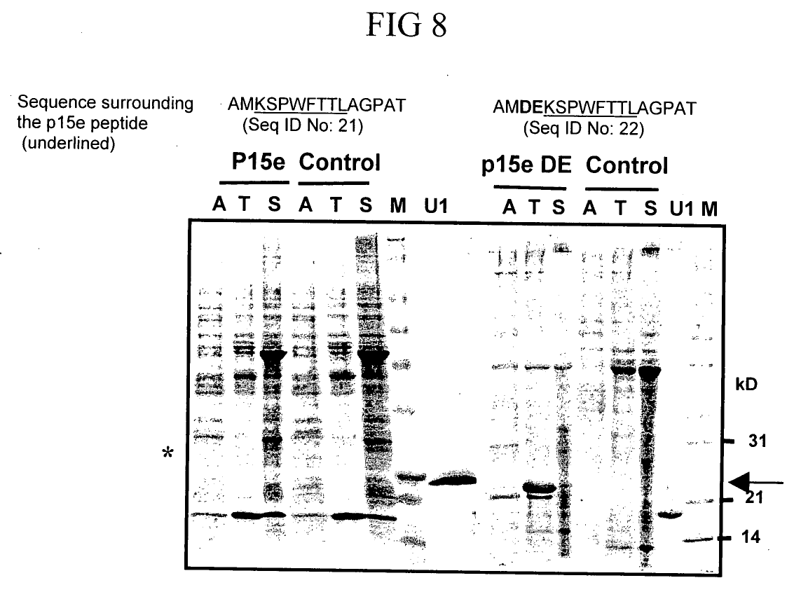

Molecular fusion of epitopes to TMV fail to accumulate when aromatic (for example W) or hydrophobic amino acids are present in the peptide. For example, p15e, a mouse melanoma antigen, contains the aromatic amino acid tryptophan (W). This peptide, when introduced onto the N or C-terminal positions on U1 coat, caused virus instability and no TMV systemic infection was observed. Applicant reasoned that to create a more favorable environment for peptide solubility, flanking amino acids could be added to increase hydrophilic interactions, counteracting the negative effects on virus assembly or stability when amino acids like W are introduced onto the solvent exposed surface of coat protein. Aspartic Acid (D) and Glutamic Acid (E) are amino acids that are charged, and were used to show that such a method will rescue the insoluble fusion of p15e to TMV coat (FIG. 8). Before addition of DE adjacent to the p15e peptide, no accumulation of product was observed (*). After addition DE to p15e, product accumulation is clearly visible (arrow). Other amino acids could also be used to alleviate negative effects of peptide composition on TMV accumulation, such as Asparagine (N), Glutamine (Q), Histidine (H), Lysine (K), Serine (S) or Threonine (T). The number and type of flanking amino acids that are sufficient to overcome negative effects on TMV expression levels or assembly may be fusion-peptide specific, and may need to be tested empirically for each peptide. This example illustrates the use of mitigating sequences to permit isolation of genetic fusions (S15, FIG. 4)

### EXAMPLE 3

### Chemically Conjugated Epitope Fusions to TMV U1

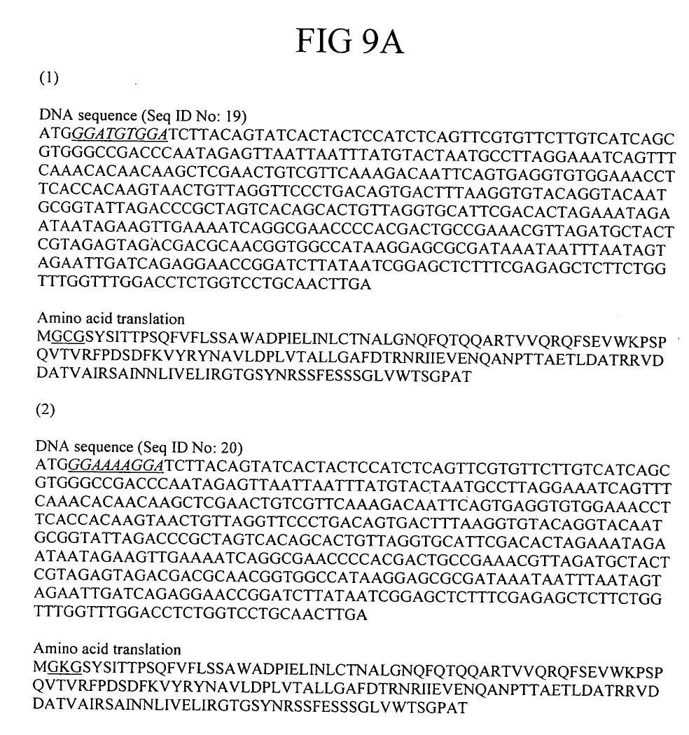

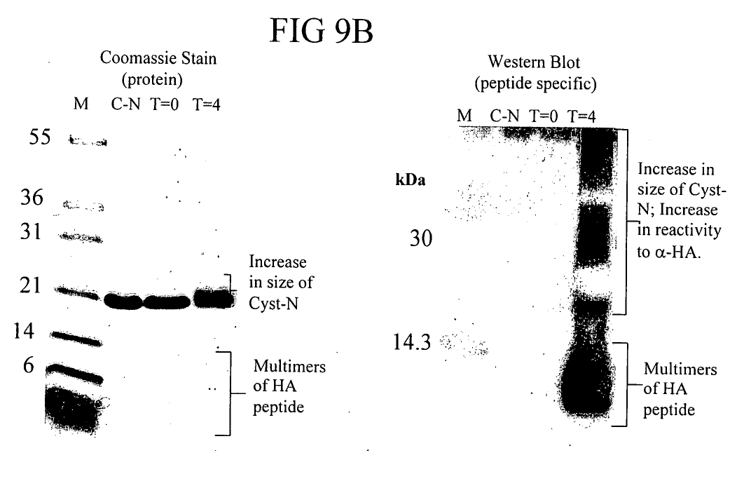

Only a percentage (70-80%; see Example 1) of genetic fusions are capable of functional VLP formation for many plant viruses. Many fusions fail to accumulate while others are simply insoluble. The present invention includes construction of coat protein fusions containing cysteine (Cys) residues as either N-terminal or surface loop fusions. The initial fusions to TMV U1, and to other tobamovirus coat proteins showing good expression in the U1 vector, are composed of glycine-cysteine-glycine (GCG) or GGCGG as N- and surface loop fusions (FIG. 9A (1)). Previous LSBC experiences have indicated that cysteine residues are tolerated on the virion surface and that under the reducing conditions of the plant cytosol, no disulfide bridges are formed between coat protein subunits or host proteins. The production of coat protein with surface exposed Cys residues allows peptide conjugation to the TMV virions through conjugation using heterobifunctional chemical cross-linking reagents, e.g. N-succinimidyl-3-(2-pyridyldithio) propionate (SPDP). SPDP allows coupling of free sulfhydryl group with a free amine group, such as that found on lysine (K), at neutral pH under mild reaction conditions. SPDP fused immuno-conjugates have been used extensively in in vivo administrations. Peptides used for initial studies and comparative biochemical response of the various tobamovirus coat proteins (CPs) are the c-myc tag (EQKLISEEDLK), the HA tag (YPYDVPDYAK) and the V5 tag (GKPIPNPLLGLDSTK). Each is synthesized (Sigma chemical) to contain a C-terminal lysine for conjugation to the sulfhydryl group. In addition to peptides, SPDP could be used to fuse the immune stimulatory single stranded DNA CpG polynucleotide using a thiolated 3′ terminus to the TMV virions as well. An alternative approach is to introduce a different reactive amino acid, such as lysine, into the region of solvent exposed residues of TMV coat protein (FIG. 9A (2)), and synthesize peptides with a C terminal or N terminal cysteine for conjugation.

Initially, SPDP conjugations are tested for reactivity to cysteine containing TMV that is not disassembled. Cross-linking reactions are carried out using short chain, long chain and sulfo-NHS forms of SPDP as described (Hermanson, G. Bioconjugate Techniques 1996 Rockford 11, Academic Press, and references therein). Peptide-SPDP adducts are mixed with cysteine TMV U1 virus and then analyzed 16 hours later for a size shift that represents physical association of the peptide with the virus. The procedure is then extended to 20S disks. An alternative approach was to use a less specific chemical conjugation strategy employing glutaraldehyde. The HA peptide was mixed with either TMV or N terminal cysteine TMV in the presence of glutaraldehyde. After a four hour incubation with glutaraldehyde, a HA peptide-TMV cysteine conjugate was formed and was visible as an increase in mass by Coomassie, as well as by an increase in apparent molecular weight by Western analysis (FIG. 9B). No such conjugate was present if wild type TMV (with no solvent exposed cysteine) was used in the conjugation reaction (data not shown). Conjugation by non-specific cross-linking agents, such as glutaraldehyde, leads to higher molecular weight aggregates as is clearly visible in the Western blot. Other conjugation reagents with more specific chemistry, such as SPDP, EDC or other heterobifunctional linkers, generate one to one or directional coat to fusion peptide chemistry, and result in more controlled conjugation reactions.

An alternative strategy is to assemble N cysteine coat into 20S discs, reassemble these discs with other discs that carry functional epitopes (ie, by molecular fusion) onto an RNA, and incubate the fully reassembled mixture with SPDP-associated peptide or moiety in order to add a new functionality. This is especially useful if the SPDP conjugation renders 20S discs chemically inert and unable to reassemble with other discs, or if the peptide that is carried interferes sterically with reassembly. As well, the ability to add a variety of agents after reassembling a monomer or a multimer has great utility. For example, SPDP conjugation of ssDNA such as CpG oligonucleotides may allow for the augmentation of immune modulation, which is greater than simply mixing the CpG with the vaccine. This could lead to better efficacy and or the potential to reduce the dose. This example illustrates the steps S3 (FIG. 1) and S9 (FIG. 2) as well as providing alternative routes to combine chemically and genetically attached epitopes.

### EXAMPLE 4

### Electron Microscopy of TMV Coat Protein Fusions

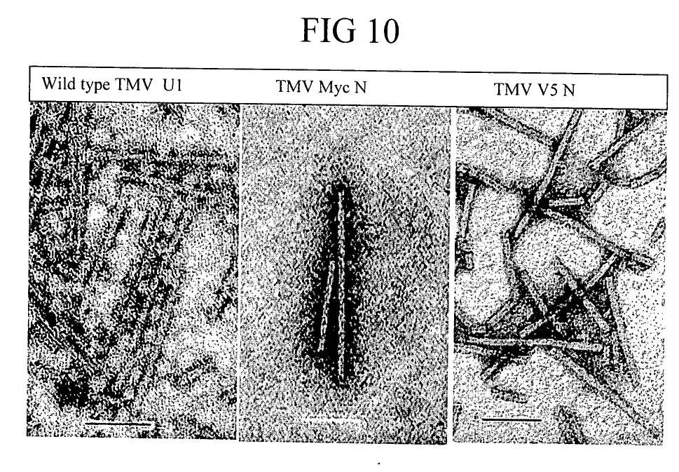

To determine the influence of the fusions on virus structure, transmission electron microscopy (TEM) was performed (FIG. 10). Wild type TMV rods have the dimensions 18-20 nm×300 nm. The N terminal epitope fusions of the model peptides V5 and Myc were visually similar the wild type U1 virus, as were the rod dimensions. This indicates that the fusion does not hinder normal coat protein reassembly in vivo and that the fusions constitute good candidates for in vitro reassembly.

### EXAMPLE 5

### Extraction and Partitioning of Wild Type TMV U1

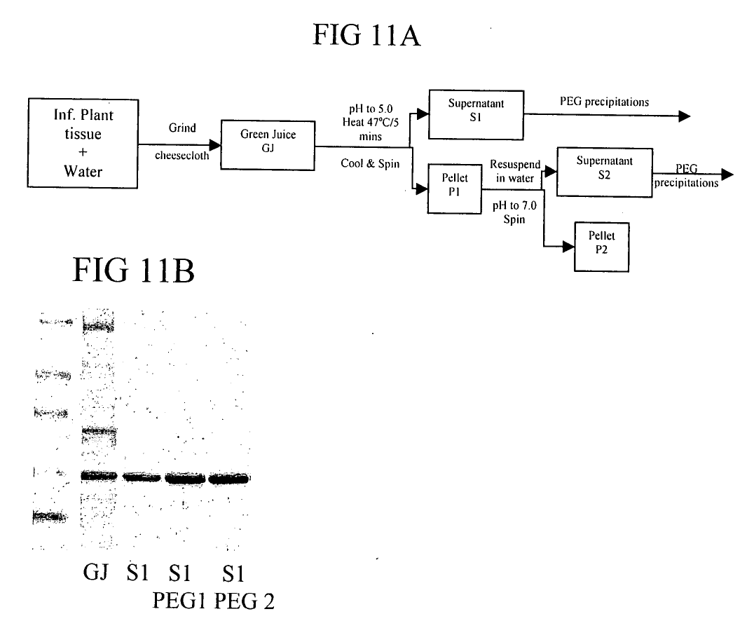

The extraction and processing of TMV U1 has been extensively discussed in the above mention commonly assigned U.S. Pat. Nos. 6,303,779, 6,033,895 and 6,037,456, which are incorporated herein by reference in their entirety. The processing is summarized in FIG. 11A. Briefly, a weighed mass of infected tissue is combined with two volumes of chilled water, containing 0.04% w/v sodium metabisulfite and grinding is preformed in a Waring blender. The homogenate is passed through 4 layers of cheesecloth to remove the fiber, leaving the green juice (GJ). The pH of the GJ is adjusted to 5.0. followed to heating to 47° C. for 5 minutes. After chilling the GJ is spun to precipitate insolubles, yielding a first supernatant. In cases where the virus partitions into the remaining pellet P1, the pellet is resuspended in water and adjusted to pH 7.0. Following a centrifuge spin the virus is recovered in a second supernatant and the final pellet P2 is discarded. To purify and concentrate the virus, two serial selective precipitations are performed on the first and second supernatants processing streams. Precipitation of the virus is achieved by adjusting the supernatants to 4% w/v polyethyleneglycol (PEG) and 4% w/v NaCl, and chilling for 30-60 minutes. Following a centrifuge spin the virus is recovered as a pellet and contaminating proteins remain in the supernatant, which is discarded.

FIG. 11B and Table 3 show representative results for wild type TMV U1 isolated from N. tabacum MD609. The SDS gel clearly demonstrates that the process yields a final virus preparation of high purity. Using BSA as a standard the coat protein bands were quantified densitometrically and a material balance for the process performed to determine recovery (Table 3). From the data it is clear that the majority of the virus partitioned into the S1 process stream and with minimal losses during the PEG precipitation a total process recovery of 76% was achieved.

TABLE 3

| mg | | | Losses | Losses | Mg virus | Total |
|---|---|---|---|---|---|---|
| virus/ | | % in | during S1 | during S1 | recovered/ | process |
| g FW | % in S1 | S2/P1 | PEG1 | PEG2 | g FW | recovery |
| 1.7 | 86% | 14% | 9% | 2% | 1.3 | 76% |

Table 3 Material balance for the isolation of TMV U1 from infected N tabacum MD609 plants. Data was generated from the densitometric analysis of the gel in FIG. 11, using a BSA standard curve.

### EXAMPLE 6

### Influence of Epitope Fusion on Virus Extraction and Partitioning

The process outlined in Example 5 was employed for a selection of the coat protein fusions listed in Table 2. Material balances were performed to determine the partitioning of the virus between the S1 and S2 process streams, in addition to the total process recovery. The identity of each fusion was confirmed by MW MALDI. The results for these purifications are summarized in Table 4. From the table it is clear that the processing characteristics are epitope fusion and location dependent. A material balance on the extraction gave initial recoveries (S1+S2 process streams) from 90-100% (e.g. HPV ep2 N) to lower than 10% (e.g. V5 N). Partitioning between the S1 and S2 streams also varied substantially. Overall recoveries also ranged from 0.5% to 79%. Based on this data the cysteine N, Myc N and V5N coat protein fusions were carried forward to optimization studies to determine conditions which would improve overall process recoveries. This optimization is detailed in Examples 7 and 8 and illustrates process modifications that can be employed in order to isolate TMV virus displaying genetic fusions (step SIS, FIG. 4).

TABLE 4

| | | | | Overall process |
|---|---|---|---|---|
| Fusion | % in S1 | % in S2 | Streams processed | recovery |
| Cysteine N | 10% | 44% | S2 | 0.5% |
| Myc #1 N | 38% | 26% | S1 and S2 | 13% |
| Myc #2 N | ˜20% | 24% | S1 and S2 | 6% |
| Myc C | N/A | N/A | S1 and S2 | 13% |
| V5 #1 N | N/A | N/A | S1 | 2% |
| V5 #2 N | ˜5% | ˜5% | S1 and S2 | 3% |
| HPV ep2 N | 60% | 40% | S1 | 51% |
| OVA N | 26% | 50% | S1 and S2 | 79% |

Table 4 Virus partitioning and overall process recovery for various coat protein fusion epitopes. Fusion location designation; N, N terminus; C, C terminus; GPAT, N terminal to GPAT sequence. # indicates the purification run number for fusions isolated more than once.

### EXAMPLE 7

### Influence of Sodium Chloride on Virus Extraction and Partitioning

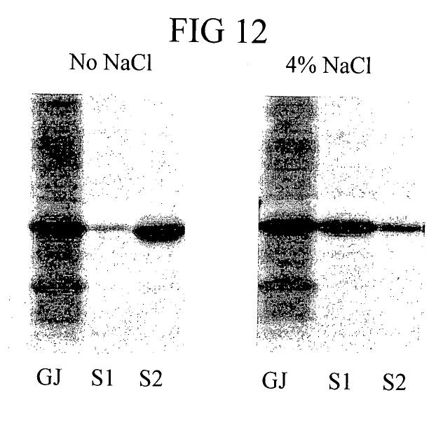

The incorporation of sodium chloride into the extraction buffer was tested as a means to improve virus recovery and alter virus partitioning. GENEWARE-infected N benthamiana plants were harvested and the biomass split, to perform a head to head comparison of extraction in the presence and absence of salt. One half of the plant material was extracted in chilled water containing 0.04% sodium metabisulfite and the remaining biomass was extracted in a 50 mM acetate buffer, pH 5.0, containing 4% w/v NaCl and 0.04% sodium metabisulfite. Processing was performed following the procedure outlined in Example 5. A comparison of the S1 and S2 fractions by SDS-PAGE, for the Cysteine N TMV fusions (FIG. 12), clearly illustrates that the presence of salt forces the virus to partition to the S1 fraction. This is favorable as the virus obtained from this stream is typically less contaminated by plant pigments and impurities. Also, from FIG. 11A it is clear that S1 partitioning is preferential to S2 partitioning as it reduces the number of processing steps.

A material balance for extractions in the presence and absence of salt is given in Table 5. From the data for Cysteine N, it is clear that the overall process recovery was improved substantially with the addition of salt; although the total virus extracted in both cases was identical, the virus loss in the absence of salt was 44% (remained associated with the P2 pellet) compared to only 7% with 4% w/v sodium chloride. Table 5 also has data for recovery and virus partitioning of the Myc N and V5N coat protein fusions during extraction. The benefits of sodium chloride are again evident, indicating that this process modification has general applicability.

TABLE 5

| | | | | | Losses |
|---|---|---|---|---|---|
| | | mg virus/g | | | during |
| Fusion | Buffer | FW | % in S1 | % in S2 | extraction |
| Cysteine N | No NaCl | 1.9 | 10% | 46% | 44% |
| | 4% w/v NaCl | 1.9 | 88% | 6% | 7% |
| Myc N | No NaCl | 3.3 | 38% | 26% | 36% |
| | 4% w/v NaCl | 2.9 | 90% | 8% | 2% |
| V5 N | No NaCl | 1.2 | ˜5% | ˜5% | 90% |
| | 4% w/v NaCl | 1.2 | 63% | 0% | 37% |

Table 5 Material balance for the isolation of viruses displaying multiple epitopes from infected N benthamiana plants. Data was generated from the densitometric analysis of the SDS gels, using a BSA standard curve.

### EXAMPLE 8

### Influence of Salt and PEG Concentration of Virus Precipitation

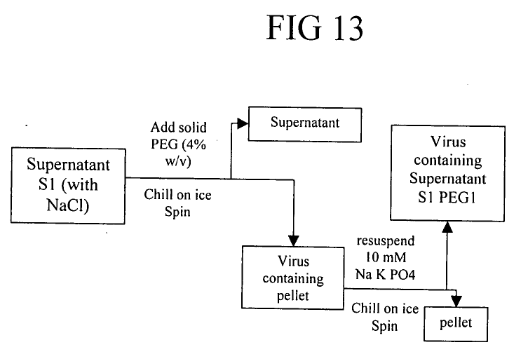

As illustrated in FIG. 11 the virus in either the S1 or S2 processing streams is further purified and concentrated by a series of two PEG precipitations. The steps involved in the first PEG precipitation are outlined in the flow diagram below (FIG. 13). The S1 (or S2) supernatant is adjusted to 4% w/v polyethylene glycol and 4% w/c NaCl. If the supernatant already contains NaCl only solid PEG is added, dissolved with agitation and the sample chilled on ice. The precipitated virus is pelleted by centrifugation and the supernatant discarded. The virus-containing pellet is then resuspended in a low ionic strength buffer and a low speed clarification spin performed. This will pellet any residual pigment and aggregated contaminating plant proteins, leaving the virus in solution. This solution is then resubmitted to a second PEG precipitation by adjusting to 4% w/v PEG and 4% w/v NaCl and repeating the process.

Table 6 compares the recoveries obtained from the two-step PEG precipitation for wild-type TMV U1 and two coat protein fusions, Myc N and V5N. The standard procedure outlined in FIG. 13 resulted in poor recoveries for both coat protein fusions compared to the wild type U1. From the flow diagram the losses can result from incomplete precipitation of the virus by the PEG, or pelleting of the virus during the clarification step. A material balance around each step in the PEG precipitation indicated that for Myc N 4% w/v PEG was insufficient to pellet the virus and the majority remained in the supernatant. In this case an increase in the PEG concentration, to 8% w/v, was required and this modification improved recovery from 6% to 60%. For the V5N virus complete precipitation was achieved with 4% w/v PEG, however, the virus failed to remain in solution during the clarification spin. By resuspending the virus-containing pellet in 10 mM Na K PO₄ containing 4% w/v NaCl, the virus remained soluble and recovery was increased from <1% to 95%. These two examples illustrate how the fusion can influence the virus properties and provide methods to maintain virus solubility during processing.

TABLE 6

| | Stream | | Losses during | Losses during | Recovery PEG |
|---|---|---|---|---|---|
| Fusion | processed | Conditions | S1 PEG1 | S1 PEG2 | precipitation steps |
| Wild type | S1 | Standard | 9% | 2% | 89% |
| U1 | | | | | |
| Myc N | S1 | Standard | 80% | 63% | 6% |
| V5N | S1 | Standard | ˜95% | ˜95% | <1% |
| Myc N | 1 | 8% w/v PEG | 25% | 20% | 60% |
| | | Resuspend in | | | |
| | | 4% w/v NaCl | | | |
| V5 N | 1 | Resuspend in | 5% | 1% | 94% |
| | | 4% w/v NaCl | | | |

Table 6 Optimization of PEG precipitation steps for TMV coat protein fusions

### EXAMPLE 9

### Generation of Free Coat Protein and 20S Disks

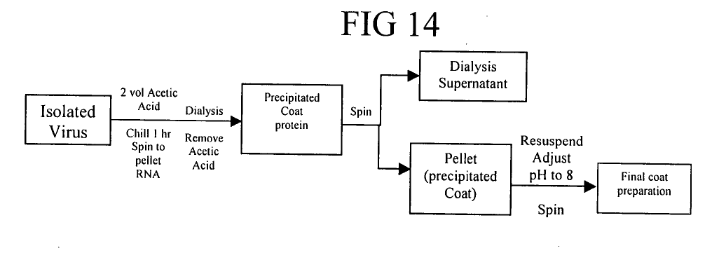

This example illustrates in greater detail steps S7 and S8 (FIG. 2) and steps S16 and S17 (FIG. 4). Coat protein was generated from purified virus using a modified version of the protocol developed by Fraenkel Conrat (Virology 1957, 4, 1-4), which is summarized in FIG. 14. Briefly the virus was combined with 2 volumes of glacial acetic acid and incubated for 1 hour at 4° C., resulting in disassociation of the virus and degradation/precipitation of the RNA. Following centrifugation to remove the degraded RNA, the acetic acid was removed by dialysis. Alternatively an ultrafiltration/diafiltration can be employed to remove the majority of the acetic acid, prior to dialysis. With dialysis the coat protein precipitates at its isoelectric point. The precipitated coat protein was isolated by centrifugation and resuspended in water. By adjusting the pH to 8, the coat protein was resolubilized and subjected to a final spin to remove any remaining aggregated species.

This process was employed to generate a number of free coat protein fusions from purified virus. Table 7 summarizes the process recoveries for a selection of the epitope fusions for which coat protein was generated.

TABLE 7

| | |
|---|---|
| Acetic Acid removal | Overall process |

| Fusion | UF/DF | Dialysis | recovery |
|---|---|---|---|
| HPV ep2 N | | + | 49% |
| ELDKWAS N | | + | 68% |
| Myc C | + | | 50% |
| Myc N | + | | 38% |
| V5 N | + | | 34% |

Table 7. Free coat protein generation for a selection of epitope fusions. Fusion location designation; N, N terminus; C, C terminus.

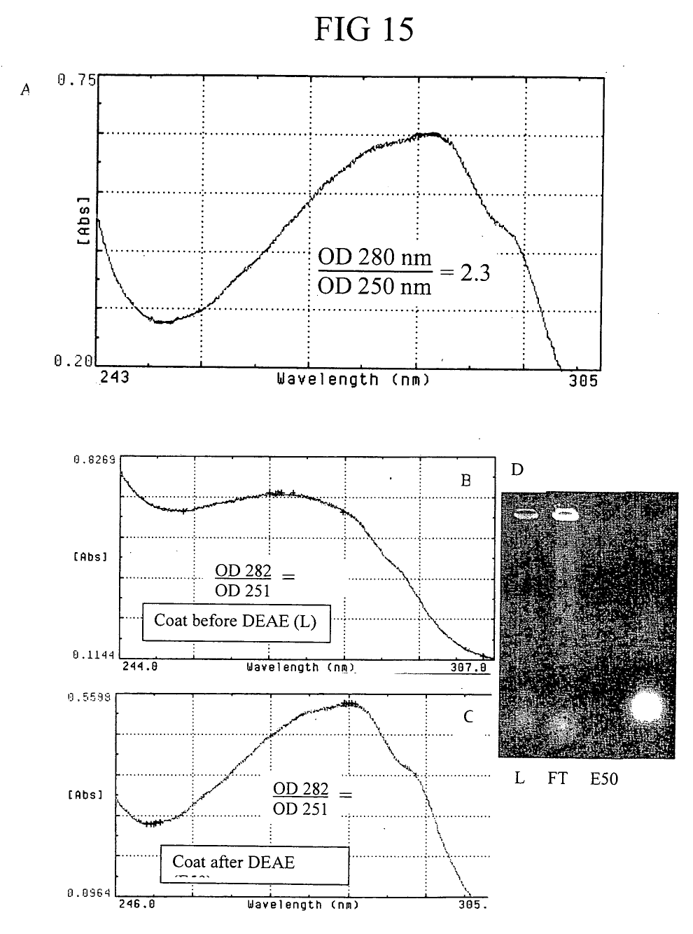

The quality of the coat protein was assessed by its ultraviolet absorption spectrum (Durham, J Mol Biol, 1972, 67: 289). The spectrum should have an absorbance maximum at 282 nm, an absorbance minimum at 251 nm and a maximum to minimum ratio between 2.0 and 2.5. A lower ratio indicates residual RNA contamination of the coat protein preparation. FIG. 15A shows the typical absorption spectrum for wild type TMV U1 coat protein. Table 8 summarizes the absorbance ratio for free coat protein preparations displaying various epitope fusions. In cases where the maximum to minimum ratio was lower than expected, e.g. Myc N, the coat protein preparation was treated with an anion exchange resin, such as DEAE Sepharose. The contaminating RNA associates strongly with the positively charged resin, while the coat protein's association will be lower, permitting selective elution of the coat protein at low chloride ion concentrations. This approach was successful at separating Myc N coat protein from contaminating residual RNA by a 50 mM NaCl elution, to yield a coat protein preparation with a maximum to minimum absorbance ratio greater then two (FIG. 15 B to D)

TABLE 8

| Fusion | OD Ratio |
|---|---|
| HPV ep2 N | 2.1 |
| ELDKWAS N | 2.2 |
| Myc C | 2 |
| Myc N | 1.22 |

Table 8 Ratio of absorbance maximum (282 nm) to absorbance minimum (251 nm) for free coat protein displaying various epitope fusions. Fusion location designation; N, N terminus; C, C terminus.

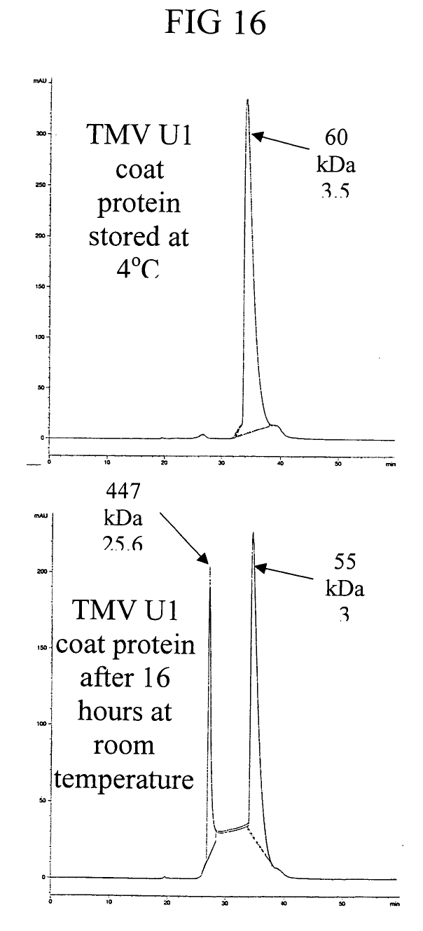

Prior to use in reassembly reactions, or even without reassembly into a whole VLP, the coat protein preparation is converted from 4 S subunits, consisting of 3 to 4 coat proteins, to 20 S disks (see FIG. 5). This is accomplished by incubating the coat protein preparation at room temperature for 24 to 48 hours prior to use, under the correct pH and ionic strength conditions. For example, TMV U1 coat protein, in 0.1 M phosphate buffer, pH 7.0 was allowed to equilibrate to room temperature (20-22° C.), from 4° C., over 16 hours and the initial and equilibrated coat preparation was analyzed by size exclusion chromatography. As seen in FIG. 16, room temperature incubation results in a bimodal distribution, resulting from the formation of 20 S disks. Alternatively, individual coat proteins may be used in lieu of the 20 S disks for mixing and reassembly.

### EXAMPLE 10

### Reassembly of Wild Type TMV Virions from 20S Disks

This Example, together with Examples 11-13, illustrate the methods for the generation of multivalent and bifunctional vaccines i.e. step S18 (FIG. 4) to yield P4 (FIG. 4). The standard conditions for TMV reassembly have been outlined for wild type U1 coat protein and a wild type TMV RNA scaffold (Fraenkel-Conrat, H and Singer, B (1959) Biochim Biophys Acta, 33, 359-370). Typically a 0.1 M phosphate or pyrophosphate buffer at a pH of 7.0 to 7.5 is employed with a mass ratio of coat protein to RNA of 22:1.

TMV U1 coat protein was generated from wild type virus isolated from N. tabacum var. MD609 plants, as described in Example 9. Wild type RNA was isolated from the same virus with the RNeasy Plant Mini Kit (Qiagen, Valencia, Calif.). Reassembly reactions were performed in 200 μl volumes, at a coat protein concentration of 1100 μg/ml and a RNA concentration of 50 μg/ml, in a 96 well plate format. The reactions were buffered with 0.1 M phosphate or pyrophosphate, pH 7.2 and the coat protein preincubated for two days at room temperature prior to use. This preincubation results in the formation of 20S disks from the 4 S subunits (FIG. 16). In addition to the standard conditions, the addition of the ribonuclease inhibitor RNasin to the 0.1 M phosphate buffered reaction was also tested. The reassembly reactions were followed by measuring the change in absorbance at 310 nm over time, which corresponds to the increase in the average length of the reassembly products.

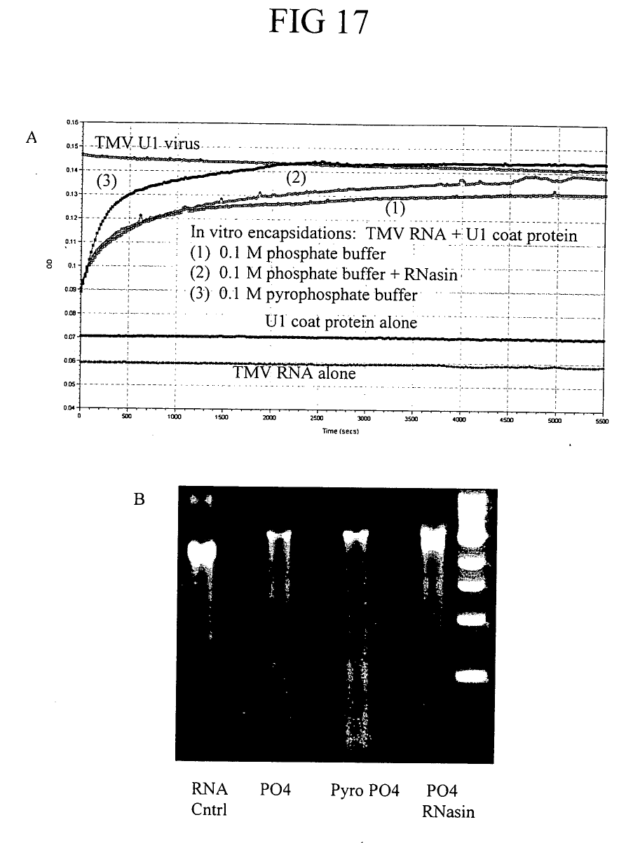

FIG. 17A shows the A310 nm profiles for the reassembly reactions. The wild type virus control was such that the molar RNA concentration was equivalent to that of the reassembly reactions. The use of pyrophosphate in place of phosphate improved the initial rate of reassembly and the OD maximum corresponded to that of the TMV virus control. For the phosphate buffered reassembly reaction the maximum OD was lower than the virus trace (0.12 OD vs. 0.14 OD). Assessing the RNA integrity in the final reassembly reaction by agarose gel electrophoresis (FIG. 17B) indicated that RNA degradation was occurring in both the pyrophosphate and phosphate samples, and to a greater extent in the latter. The addition of different ribonuclease inhibitors to the coat protein was therefore tested. The ribonuclease inhibitor (either RNasin (Promega, Madison, Wis.) with and without additional DTT, or SUPERase (Ambion, Austin, Tex.)) was added to the coat protein preparation 30 minutes prior to RNA addition (0.2-4 U/ul). SUPERase at all concentrations tested was ineffective whereas the RNasin reduced RNA degradation substantially (FIG. 17B). The presence also improved the maximum OD 310 nm attained for the reassembly reaction (FIG. 17A).

To determine the functional significance of the different buffer combinations, aliquots of the reassembly reactions were analyzed by the local lesion host assay (Table 9). The reassembly reactions, naked RNA and virus controls were serially diluted and applied to the leaves of N tobacum ‘Xanthi’ NN plants, with carborundum employed as an abrasive. Five days post inoculation the lesion numbers were counted and provided a semi-quantitative measure of the titer of functional virus in the reassembly reactions.

TABLE 9

| | | Free | | | |
|---|---|---|---|---|---|
| | Virus | RNA | Reassembly | Reassembly | Reassembly |
| Dilution | control | control | PO4 | pyro PO4 | PO4 RNasin |
| 10-2 | | 45 ± 13 | | | |
| 10-3 | | 4 ± 1 | | | |
| 10-4 | 123 ± 41 | 1 ± 1 | 25 ± 7 | 95 ± 31 | 122 ± 44 |
| 10-5 | 14 ± 10 | | 6 ± 4 | 3 ± 1 | 19 ± 7 |

Table 9 Local lesion host assay data for reassembly reactions with wild type U1 coat protein and TMV RNA. PO4, 0.1 M phosphate buffered; pyro PO4, 0.1 M pyrophosphate buffered; PO4 RNasin, 0.1 M phosphate buffered with 0.4 U/μl RNasin ribonuclease inhibitor.

Comparing the infectivity of free RNA to the reassembly reactions, which contained an equivalent molar concentration of RNA, clearly illustrates the improvement in infectivity with RNA encapsidation. Within the reassembly reactions, a marked improvement in infectivity was evident for the phosphate buffer when RNasin was present, which correlated with the improvement in RNA integrity and A310 nm OD maximum. The observed infectivity with RNasin was comparable to that of the virus control. The pyrophosphate buffer also improved infectivity due to the accelerated reassembly, which aided in the protection of the RNA.

### EXAMPLE 11

### Reassembly of Coat Protein Fusions onto TMV RNA

A central aim of this work is the generation of a multifunctional TMV-based reassembly product, which displays epitopes with different functionalities e.g. a cell targeting or immunomodulation sequence together with an antibody or CTL target. As a first step, the ability of various coat protein fusions to reassemble onto TMV RNA was examined. The fusions chosen were ELDKWAS and HPV ep2 at the N terminus and Myc at the C terminus. The reassembly reactions were performed in 200 μl volumes, at a coat protein concentration of 1100 μg/ml and a RNA concentration of 50 μg/ml, in a 96 well plate format. The reactions were buffered with 0.1 M phosphate, pH 7.0 and the coat protein preincubated for two days at room temperature prior to use. In a subset of the reactions the ribonuclease inhibitor RNasin was incorporated. The reassembly reactions were followed by measuring the change in absorbance at 310 nm over time, which corresponds to the increase in the average length of the reassembly products.

A number of other peptides could be used to enhance CTL targeting. T-cell targeting is particularly preferred. However, should one wish to suppress an unwanted preexisting immune response (allergy, autoimmune disease, etc.), one may wish to target T-suppressor cells or other cells of the immune system.

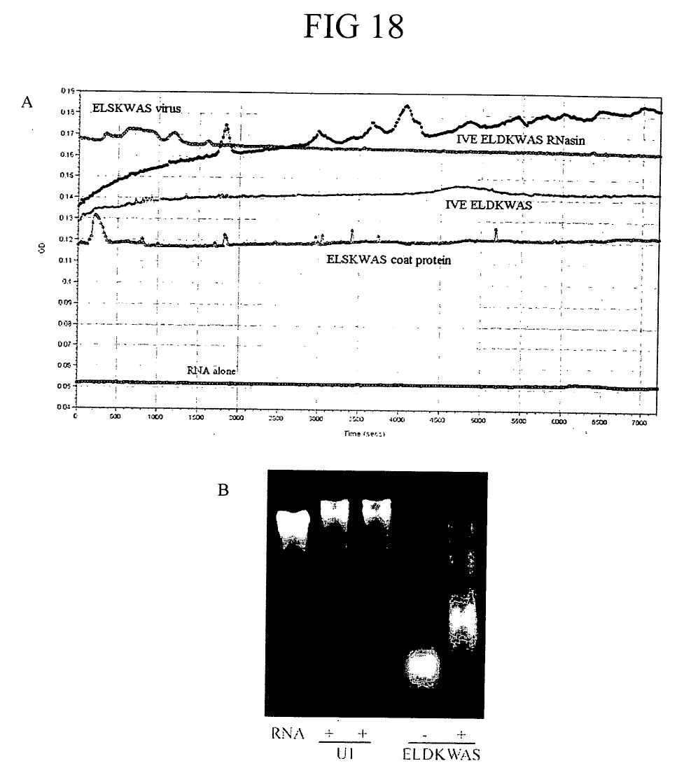

FIG. 18A shows the A310 nm profiles for the reassembly reactions involving the ELDKWAS coat protein fusion. The presence of RNasin in the reaction mixture clearly resulted in an improved absorbance profile with a higher final OD. The RNA integrity of the reassembly reactions was assessed by agarose gel electrophoresis (FIG. 18 B). Although the extent of degradation was substantially higher than for the reassembly reactions involving U1 coat protein, the presence of RNasin did reduce the extent of RNA degradation in the ELDKWAS coat protein reassemblies.

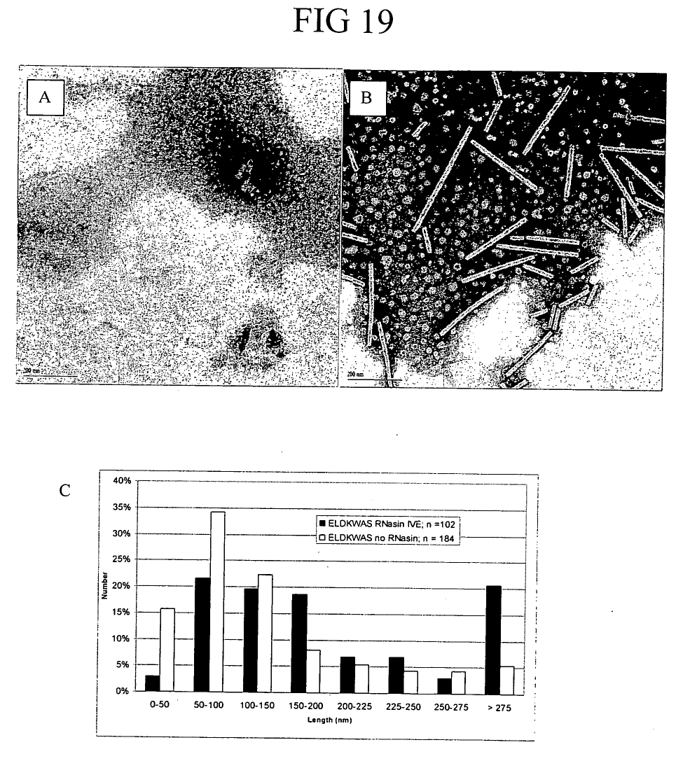

The A310 nm kinetics together with the RNA profile suggest that RNasin increases the proportion of full-length rods formed during reassembly. To confirm this, samples were analyzed by electron microscopy (FIG. 19). Comparing the images for coat protein in the presence and absence of RNA shows that reassembly of the ELDKWAS coat protein fusion onto the TMV RNA scaffold occurred. To assess the influence of RNasin, the normalized particle size distribution, obtained from the electron microscopy images was determined. With RNasin present there was a reduction in the 0-100 nm length rods with a concurrent increase, from 5% to 20% of full length (>275 nm) rods, which correlates with the A310 nm absorbance data.

The reduction in full-length rods presumably results from the reduced pool of full length RNA. This would be expected to reduce the number of functional i.e. infectious reassembly products. Analysis of the reassembly products by the local lesion host assay confirmed this reduction; omission of RNasin reduced the average number of lesions observed by a factor of 9 (Table 10).

TABLE 10

| | | Reassembly | Reassembly PO4 |
|---|---|---|---|
| Coat protein 1 | Coat protein 2 | PO4 | RNasin |
| ELDKWAS (N) | — | 4 ± 6 | 37 ± 16 |
| Myc (C) | — | 2 ± 2 | 31 ± 17 |
| HPV ep2 (N) | — | 2 ± 1 | 11 ± 6 |
| ELDKWAS (N) | HPV ep2 (N) | | 6 ± 4 |
| ELDKWAS (N) | Myc (C) | | 21 ± 13 |
| HPV ep2 (N) | Myc (C) | | 50 ± 43 |

Table 10 Local lesion host assay data for reassembly reactions with multiple coat protein fusion and TMV RNA. PO4, 0.1 M phosphate buffered; PO₄ RNasin, 0.1 M phosphate buffered with 0.4 U/μl RNasin ribonuclease inhibitor. All dilutions were at 10⁻³. At this dilution no lesions were detected for free wild type RNA. N, N terminal fusion; C, C terminal fusion.

Reassembly reactions were also performed with the HPV ep2 and the Myc coat protein fusions, in the presence or absence of RNasin. Similar to the ELDKWAS coat protein fusion, the presence of RNasin during the reassembly resulted in A310 nm profiles with a higher final OD and improved RNA integrity. From a functional standpoint the reassembly products generated in the presence of RNasin showed greater activity by the local lesion host assay (Table 10). For Myc and HPV ep2 the average number of lesions were 15 and 6 fold higher respectively when RNasin was present. These infectivity studies clearly illustrate the ability of a TMV coat protein carrying a solvent exposed epitope to reassemble and encapsidate a functional RNA.

The coat protein preparations do have a plant-derived ribonuclease activity associated with them, which can be partially mitigated by the inclusion of RNasin in the reassembly reaction. Alternative approaches can also be used to reduce the ribonuclease activity associated with the starting virion preparations, from which the coat protein preparations are generated. The virus preparation can be treated with bentonite, which inhibits ribonuclease activity (Jacoli, G., Ronald, W., and Lavkulich, L.: Inhibition of Ribonuclease Activity by Bentonite, Can J Biochem 51, 1558, 1973). Alternatively the virus preparation can be treated with diethylpyrocarbonate (DEPC) at 0.05%-0.1% v/v, which inactivates RNases by reacting specifically with the histidine residues in the enzymatic site. Residual DEPC is removed by dialyzing the treated virus extensively against any buffer containing a primary amine group, e.g. Tris (2-amino-2-hydroxymethyl-1,3-propanediol), with which DEPC reacts.

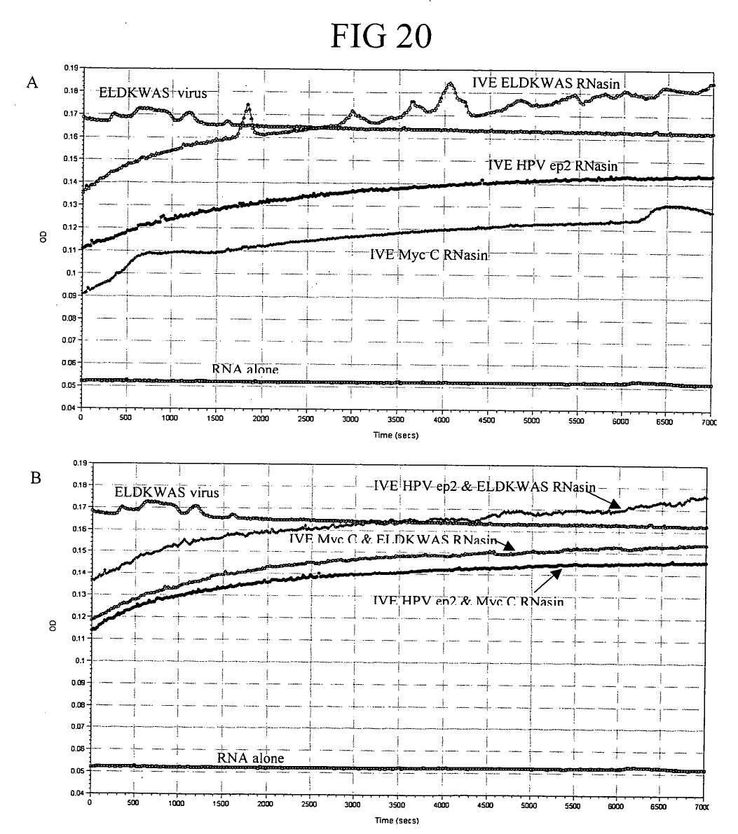

Reassembly reactions to generate a multivalent TMV-based vaccine were performed using a TMV RNA scaffold. The ELDKWAS, Myc and HPV ep2 coat protein fusions were combined pair wise at a 1 to 1 ratio. FIG. 20 compares the A310 nm reassembly kinetics for the bivalent encapsidations to those for the coat protein fusions used individually. The bivalent reactions showed a similar rise in absorbance over time indicating that reassembly was occurring efficiently in the presence of two independent coat protein fusions. To test for the generation of functional bivalent reassembled virions, local lesion host assays were performed (Table 10). Lesion numbers comparable to the monovalent assemblies were obtained, confirming the presence of functional reassembly products.

### EXAMPLE 12

### Multivalent Papillomavirus Prophylactic Vaccine

Introduction

Animals may be protected against infection with papillomaviruses by vaccination with either or both papillomavirus structural proteins, L1 and L2 (Da Silva D M et al., 2001, Journal of Cellular Physiology 186:169-182; Koutsky L A et al., 2002, New England Journal of Medicine 347:1645-51). Protection against papillomavirus infection primarily requires a specific humoral response, which results in production of virus neutralizing antibodies (Nab) directed at epitopes in the structural proteins. A cellular immune response directed against the structural proteins may also contribute to vaccine-induced immunity. Live recombinant virus and DNA vaccine vectors carrying L1, or one or more of the non-structural genes E1, E2, E4, E6, E7 and E8, can induce protective immunity in vaccinated animals; in these cases both cellular and humoral immune responses are detected (Sundaram P et al., 1997, Vaccine 15:664-71; Moore R A et al. J Gen Virol 20:2299-301). It is well established that a humoral response directed against papillomavirus structural proteins is both necessary and sufficient for protective immunity against papillomavirus infection (Embers et al., 2002 Journal of Virology 76:9798-9805). A cellular immune response against virus-encoded proteins will enhance the level and robustness of the protective immune response, but will not prevent initial infection (Tobery T W et al., 2003, Vaccine 21: 1539-47).

Bivalent or Multivalent Reassembled Vaccines

The most important papillomavirus Nabs bind conformational epitopes in L1, and recognize only intact virus, or correctly assembled virus-like particles (VLP). These Nabs recognize epitopes in hypervariable loops on the capsid surface, and generally will only neutralize closely related papillomavirus types. Antibodies that bind linear epitopes in the N-terminal region of L2 may also neutralize virus infectivity. Most importantly, Nabs directed against L2 epitopes show the ability to cross-neutralize distinct viral strains (Embers M E et al., 2002; Journal of Virology 76:9798-9805; Kawana Y et al., 2001, Journal of Virology 75: 2331-2336; Kawana K et al., 1999, Journal of Virology 73:6188-6190; Kawana K et al. 2001, 1496-1502; Roden R B S et al., Virology 270:254-257). Embers et al. (2002; Journal of Virology 76:9798-9805) demonstrated that peptides that represent linear epitopes in the L2 proteins of the rabbit papillomaviruses rabbit oral papillomavirus (ROPV) and cottontail rabbit papillomavirus (CRPV) could induce good protective immunity against challenge with the homologous virus, but not against the heterologous virus.

Recombinant TMV U1 that display the linear, neutralizing rabbit papillomavirus epitopes CRPV L2. 1; CRPV L2.2; ROPV L2.1 and ROPV L2.2 (Embers M E et al., 2002 Journal of Virology were constructed (Table 2). Each recombinant virus will induce neutralizing antibodies that will protect animals against challenge with high titer of homologous virus. However, each vaccine may not induce sufficient titer of Nabs to neutralize the heterologous virus.

Assembling at least two different coat proteins, each of which displays a different peptide, on a structural RNA that contains the TMV OAS, can make a multivalent recombinant vaccine that will induce protective immunity against both CRPV and ROPV. For example, the methods described in Example 9 may be used to isolate free coat protein from recombinant TMV virions that display the CRPV L2.1 peptide at the “GPAT” position proximal to the C terminus of TMV U1. Likewise, free coat protein may be isolated from recombinant TMV virions that display the ROPV L2.1 peptide at the “GPAT” position proximal to the C terminus of TMV U1. Wild type TMV RNA, or a recombinant RNA that contains that TMV U1 origin of assembly sequence (OAS) may be used as the scaffold on which the reassembled bivalent vaccine is built, according to methods described in Example 10 and 11. Similarly, additional recombinant U1 coat proteins that display peptides with the ability to induce Nabs in vaccinated animals may be incorporated into the reassembly reaction to generate a multivalent vaccine virus or virus-like particle. Animals that are vaccinated with bivalent or multivalent vaccines will produce antibodies that recognize the various peptide antigens fused to the recombinant vaccine molecule; these antibodies are capable of neutralizing both CRPV and ROPV. New Zealand white rabbits will thus be protected against infection against two distinct virus species after vaccination with a single vaccine moiety.

Multifunctional Vaccine: Induction of Humoral and Cellular Immunity

The sequences of human papillomavirus type 16 L2 that are homologous with the CRPV L2.1, ROPV L2.1; CRPV L2.2 and CRPV L2.2 peptides are capable of binding to specific receptors, and on binding to the cell surface are able to mediate cellular entry of proteins fused to these sequences by receptor-mediated mechanisms (Kawana Y et al., 2001 Journal of Virology 75: 2331-2336; Yang et al. 2003, Journal of Virology 77:3531-3541). It is thus expected that virions and reassembled virus-like structures that display these sequences will be able to bind to the surface of rabbit cells, and mediate entry of the reassembled virus structure into the cell. The additional cell fusion function of reassembled particles with one or more of the CRPV L2. 1, ROPV L2. 1; CRPV L2.2 and CRPV L2.2 peptides displayed by the assembled virus or virus-like particles allows delivery of a functional RNA payload to the cytoplasm of transduced cells.

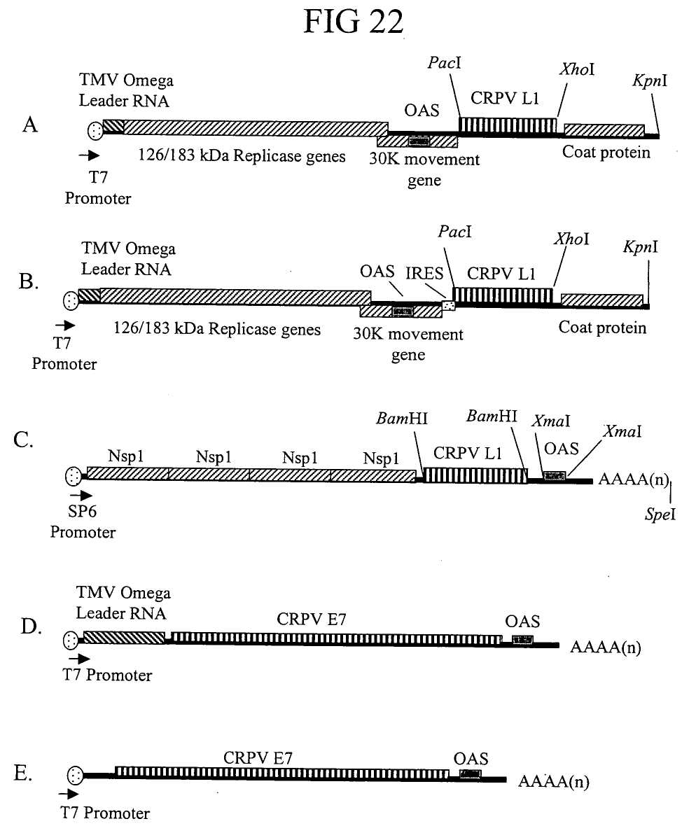

To augment the protective antibody mediated immunity induced by the L2 peptides displayed on the surface of the reassembled viral structure, the RNA scaffold will have additional biological activity. For example, the scaffold RNA is a recombinant RNA molecule that encodes the Semliki forest alphavirus (SFV) RNA sequences that are required for autonomous replication, with the CRPV L1 gene that may be expressed under the control of the 26S RNA promoter from SFV, and the TMV U1 OAS inserted downstream of the CRPV L1 gene. This construct is shown in FIG. 22. Animals are immunized with reassembled virus structures that contain the capped SFV::CRPVLI::OAS RNA molecule as a scaffold, protected by recombinant TMV coat proteins that display one or more of the CRPV L2.1, ROPV L2.1; CRPV L2.2 and CRPV L2.2 epitopes assembled on the scaffold RNA. The recombinant TMV coat proteins perform several important functions: (1) they protect the recombinant SFV RNA molecule from nuclease digestion; (2) they form a particulate, quasicrystalline structure, such as are preferentially recognized and engulfed by macrophages, dendritic cells, and other antigen presenting cells; (3) through specific cell-binding activity, they deliver the recombinant particles to the cytoplasm.

Once the particles are in the cytoplasm, the recombinant RNA molecule is translated, and the RNA undergoes one or more cycles of replication mediated by the SFV non-structural proteins (NSP) replicase activity. The subgenomic RNA encoding the CRPV L1 RNA and TMV OAS is transcribed and the CRPV L1 RNA translated. The intracellularly expressed L1 protein is then available for processing and presentation via MHC Class I to T-cells, thereby priming a cellular immune response against the L1 protein. Replication of the recombinant SFV RNA delivered to the cytoplasm of transduced cells induces the innate immune response, via pathogen surveillance signaling molecules such as the dsRNA-induced protein kinase (PKR), resulting in secretion of inflammatory cytokines such as interferon gamma. This augments the specific cellular immune response induced against the L1 ORF. Thus, a broad, robust immune response against both structural proteins (L1 and L2) is induced. Rabbits vaccinated with these multifunctional vaccines are protected against challenge with both CRPV and ROPV viruses.

An alternative method to generate a functional RNA is to insert an IRES and coding sequence for L1 into TMV RNA, which also expresses a molecular fusion of L2 peptide epitope onto coat protein. This method has the advantage of encapsidating the RNA in vivo, and does not rely on reencapsidation to protect the RNA from degradation until after cellular uptake mechanisms allow for transcription of the gene. In a third strategy, the coat protein also carries an N terminal cysteine for conjugation of a T-helper epitope, a cell fusion epitope, an adjuvant, or the full-length gene product of a non-structural protein such as E7.

Papillomavirus nonstructural proteins, including E1, E2, E4, E6, E7 and E8 are, known to mediate protective immunity, or lesion regression and clearance in vaccinated animals (Han R et al., 2002, Cancer Detect Prev 26:458-67; Han R et al., 2000. Journal of Virology 74: 9712-6). In the same manner as described above, mRNAs or autonomously replicating RNAs encoding other papillomavirus proteins which are known to mediate protective immunity, and which can induce regression or cure of virus infection, may be encapsidated within virus structures (FIG. 22).

### EXAMPLE 13

### Multivalent Melanoma Vaccine

Melanoma antigens that stimulate good protection against tumor growth are typically characterized as CTL epitopes. CTL responses are highly dependant upon the context for antigen presentation, including immunostimulation during vaccine presentation to the immune system. This is characterized by a need for either immunostimulatory cytokines, such as GM-CSF or IFNγ, adjuvants that specifically activate T cells, such as CpG oligo, or immunomodulatory peptides or proteins, such as Il1B or MIP1a or IP10, to be delivered along with the vaccine, or fused directly to the vaccine product. Melanoma CTL epitope fusions, either molecular or chemical conjugates, are reassembled onto wild type TMV RNA and tested for appropriate stimulation of peptide specific CTL responses. The same melanoma CTL epitope fusions are then reassembled onto an RNA that contains both a TMV origin of assembly, and an animal translatable codon for IFNg, GM-CSF, MIP1a, or IP 10. After vaccination with epitope TMV or epitope TMV/IFNγ (for example), the level of CTL response is measured and compared. Translation of the functional RNA produces a protein that results in immune activation, thereby increasing the CTL response.

Alternatively, the RNA encodes a second full-length antigen that primes the cellular or humoral immune response for broader immune coverage. For example, melanoma tumors express several specific antigens that generate both CTL and antibody responses in challenged individuals. In murine tumors, such antigens include p15e, tryrosinase and GP100. Several CTL epitopes, as well as antibody stimulating domains, exist for each tumor specific antigen. Defined CTL epitopes, e.g. the p15e CTL epitope, are fused to the surface of TMV, and the encapsidated RNA encodes the entirety of gp100, or tyrosinase coding sequences. CTL reactivity to the p15e epitopes is measured, and further cellular or humoral reactivity to the gp 100 or tyrosinase epitopes encoded by the RNA demonstrate RNA expression and activity of the resulting gene product.

Cellular or humoral assays indicate the level at which the vaccine is stimulating an immune response. Another way to show immune reactivity is by challenging animals with the tumor encoding those antigens and monitoring the rate of tumor growth, or the morbidity that that tumor causes. Such models exist for melanoma, and are widely used to prototype the effectiveness of melanoma vaccines. The B16 melanoma model expresses p15e, tryosinase, and gp100, and requires an effective CTL response after vaccination to reduce or eliminate the rate of tumor growth. Animals vaccinated with CTL epitope fusion vaccines are challenged with tumor, and an effective immune response will decrease the rate of tumor growth or morbidity compared to controls. If either an immunostimulatory RNA or full-length gene product encapsidated by a TMV coat or a TMV coat fusion is effective, then the rate of tumor growth should decrease compared to a protein vaccine alone, or the overall morbidity should decrease. These finding will corroborate the cellular and humoral response data, considering that these responses are essential to reducing or eliminating tumor.

As described above for papillomavirus applications, the functional encapsidated RNA can be self-replicating, such as an engineered alphavirus containing a TMV origin of assembly, or can contain an IRES, to stimulate translation from an internal site in TMV RNA (see FIG. 22 for examples). Also as described above, the combination of epitope fusions can be made either by molecular or chemical conjugation methods, and need not be limited to peptides. DNA sequences and whole proteins may also be added to reassembled TMV, or to TMV coat fusions that also encode an N-terminal cysteine.

### EXAMPLE 14

### Immunogenicity of TMV Coat Protein Fusions

Immunogenicity to V5 and Myc U1 Coat Fusions: Responses to Antibody Epitopes

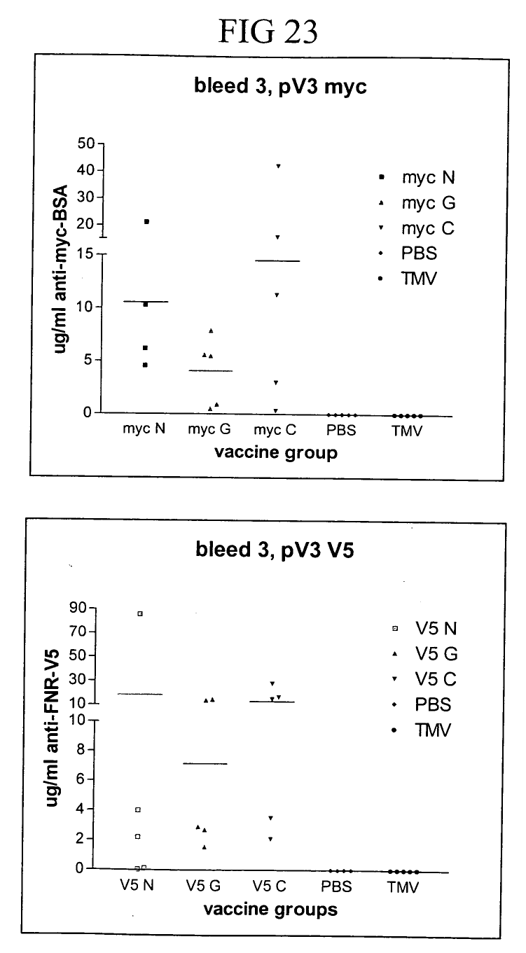

To verify that coat fusion peptides can stimulate appropriate immunity, we tested myc and V5 U1 peptide fusions, with known antibody binding properties, as vaccines in mice, and then looked for anti-myc and anti-V5 antibody responses. V5 and myc TMV U1 coat fusions were prepared by extraction methods, optimized for the recovery of the fusion of interest. Material was quantitated by the BCA protein assay, evaluated for peptide integrity by MALDI-TOF and for purity by SDS-PAGE. 10 μg of TMV protein was then injected into Balb-C mice three times, every two weeks. After the second and third vaccines, animals were bled and sera was collected and analyzed for peptide specific reactivity by ELISA. The results of serum titers after the third vaccination are show in FIG. 23, and are boosted from levels observed after two vaccines.

Results from this study indicate that at all three positions, V5 and myc peptide fusions to TMV U1 coat can elicit the appropriate anti-peptide antibody response even when given without adjuvant. Varied response levels in individual mice are typical of subunit vaccines, and have been observed for other antigen vaccines. Overall, the average response in each vaccine group tested was not significantly different by position of the peptide fusion, even though the maximum response levels differed significantly in each group. Interestingly, responses to the TMV carrier were generally lower in magnitude than the anti-peptide response (data not shown). Of note, these vaccines were administered without adjuvant, and the high levels of responses in each group show that the viral carrier can provide humoral immune stimulation that is antigen specific.

CTL Response Assay Development for Ova Peptide U1 Coat Fusions

In addition to testing the ability of antibody-target peptides to stimulate appropriate humoral responses in vaccinated mice, we also tested the ability of a CTL epitope, derived from the chicken ovalbumin protein, to stimulate appropriate cellular immunity in appropriately MHC restricted mice. 20 μg Ova-N or Ova-G TMV fusions were administered 4 times every two weeks without adjuvant to mice, and then spleens were harvested from vaccinated animals five days after the final vaccine. Cells were isolated, cultured with either media or media plus ova peptide for 5 hours in the presence of the Golgi transport inhibitor Brefeldin A, and then cells were fluorescently stained with FITC conjugated antibodies against surface expression of CD4 and CD8 T cell receptors, in conjunction with PE staining of the intracellular cytokines IFN gamma or TNF alpha. Stimulation with ova peptide should upregulate these cytokines in T cells that are specific for the peptide, and be measured by an increase in cell number by Fluorescence Activated Cell Sorting (FACS). 5×10⁵ events were collected, about 20% of which are T cells.

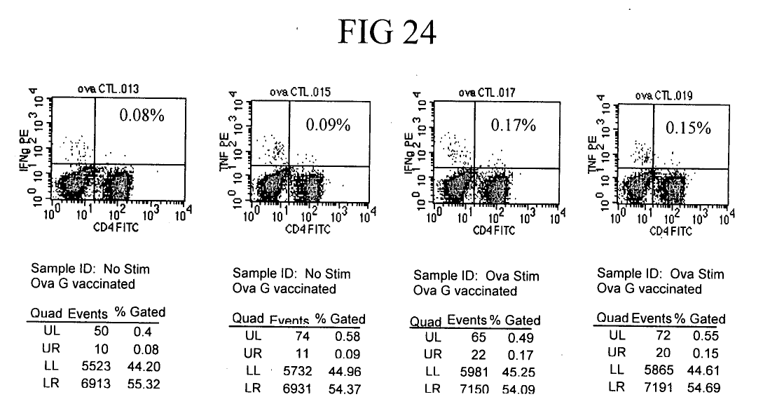

Both CD4 and CD8 cells were monitored for increased intracellular expression of IFN γ (gamma) and TNF α (alpha). As shown in FIG. 24, after a five-hour peptide stimulation, intracellular IFN gamma levels rose in CD4 positive cells (from 0.08% of gated events to 0.17% or 10 to 22 cells), and in CD8 positive cells (from 0.08% of gated events to 0.13% or 11 to 17 cells; data not shown), which represent statistically significant increases. TNF alpha levels rose significantly in CD4+ cells (0.08 to 0.13%) but did not change in CD8+ cells (0.12% to 0.10%; data not shown).

Considering that no adjuvant was administered with the vaccine, these modest increases in cytokine levels suggest that the vaccine is stimulating an appropriate cellular response. Administration of an adjuvant with the vaccine, or the fusion of immunostimulatory peptides to the TMV vaccine, is expected to increase the percentage of activated T cells. For example, the T cell activating adjuvant, single stranded CpG DNA oligo 1758, specifically augments cellular responses in ova and other CTL systems. For our system the nucleotides are either mixed with the vaccine, or fused directly to TMV U1. In other systems, the IL1b peptide has been shown to augment both antibody and CTL responses but only if the IL1b peptide is physically linked to the ova peptide vaccine, such as in a multivalent vaccine.

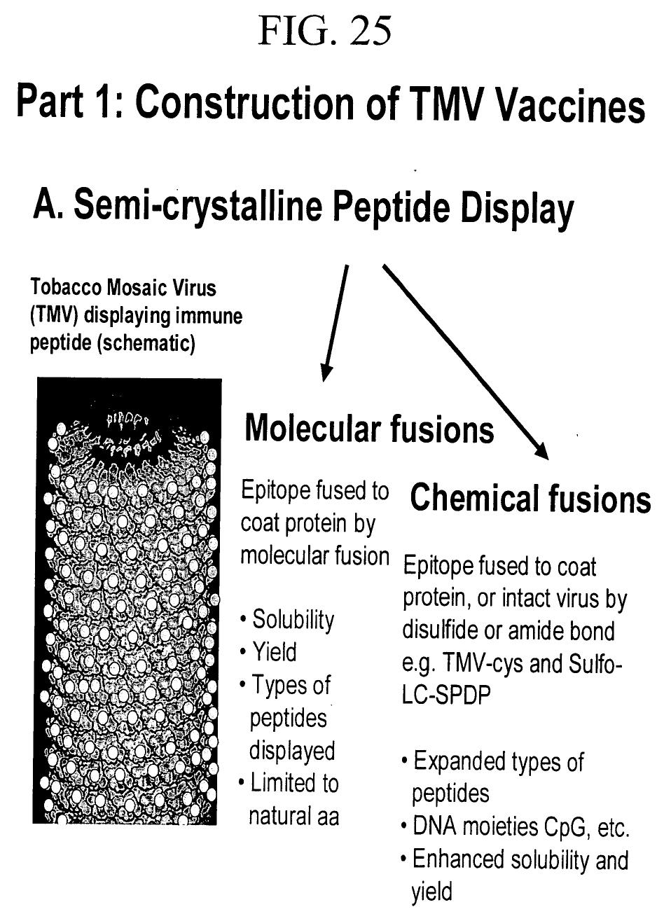

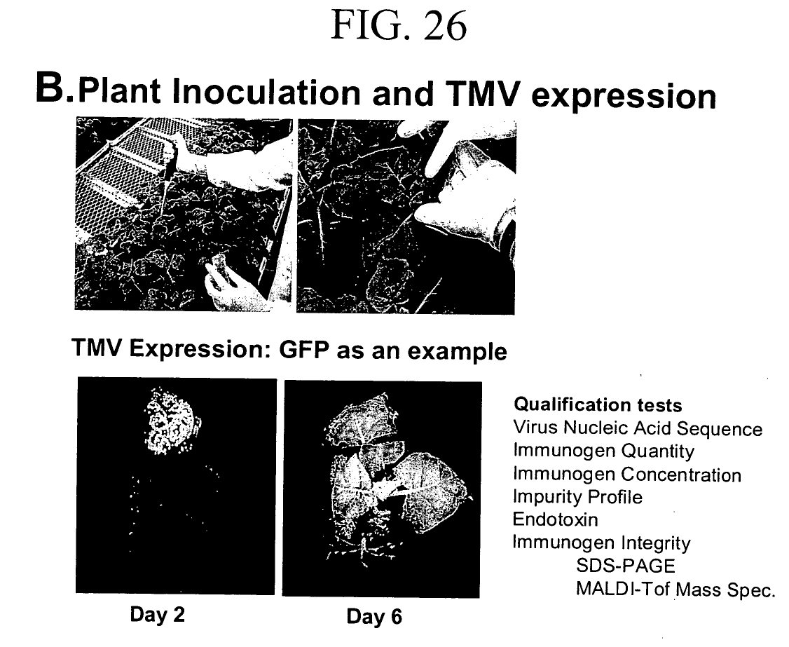

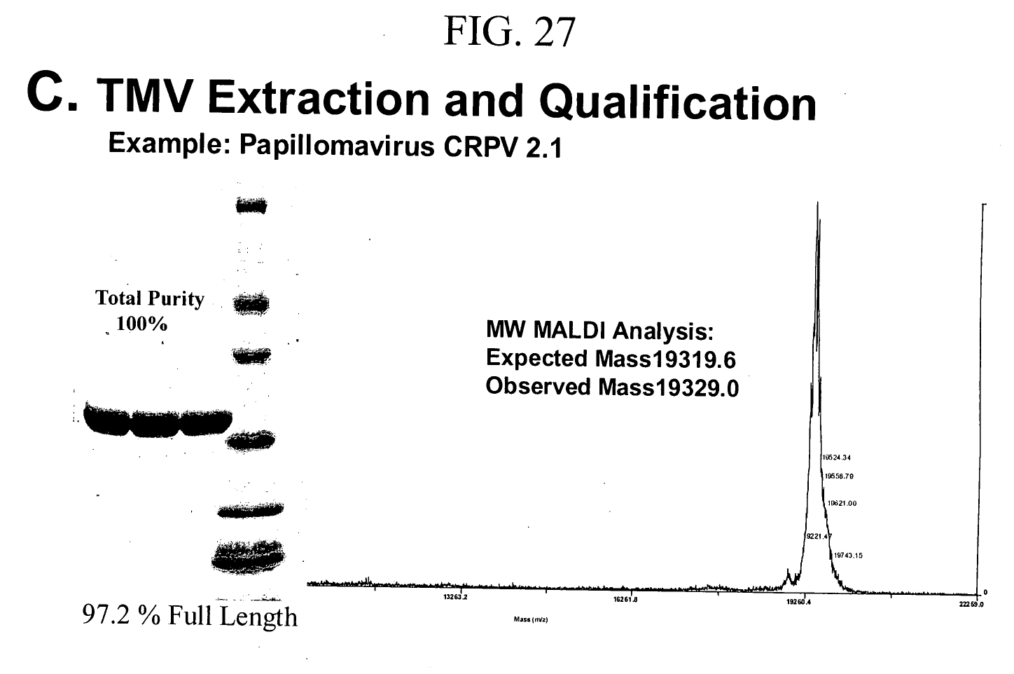

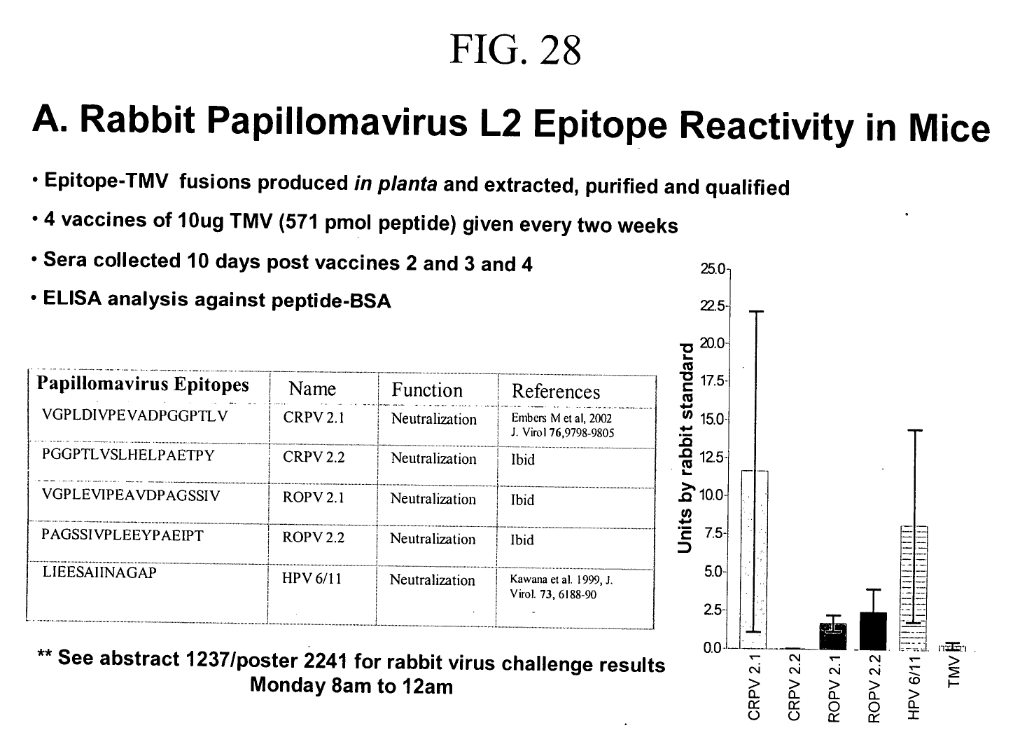

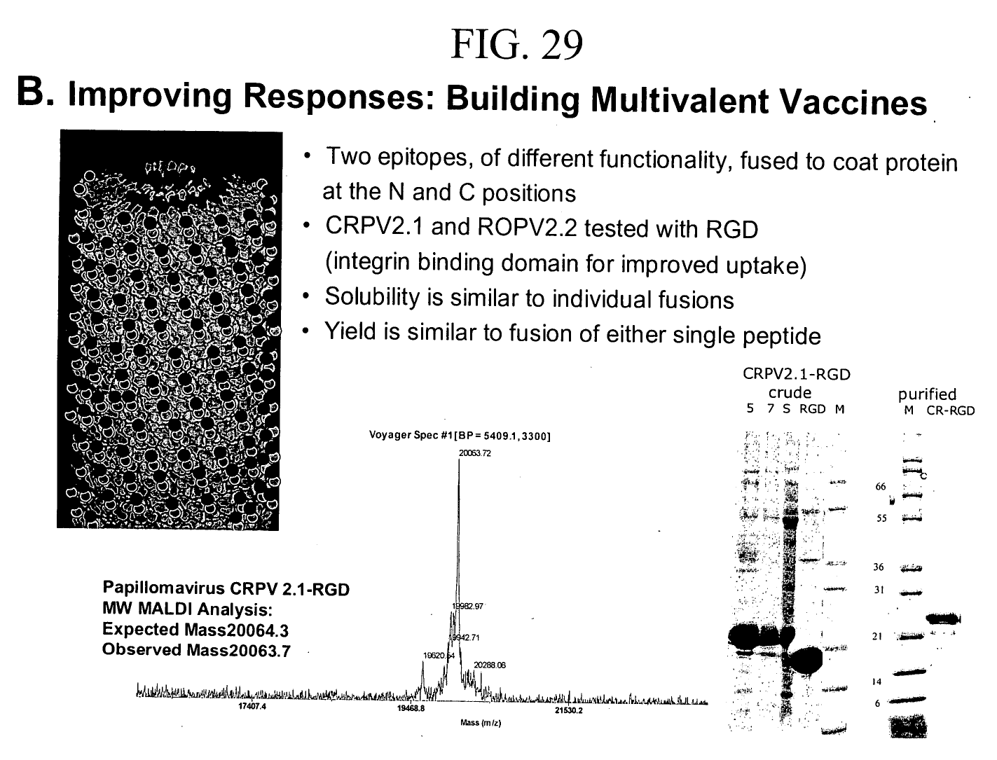

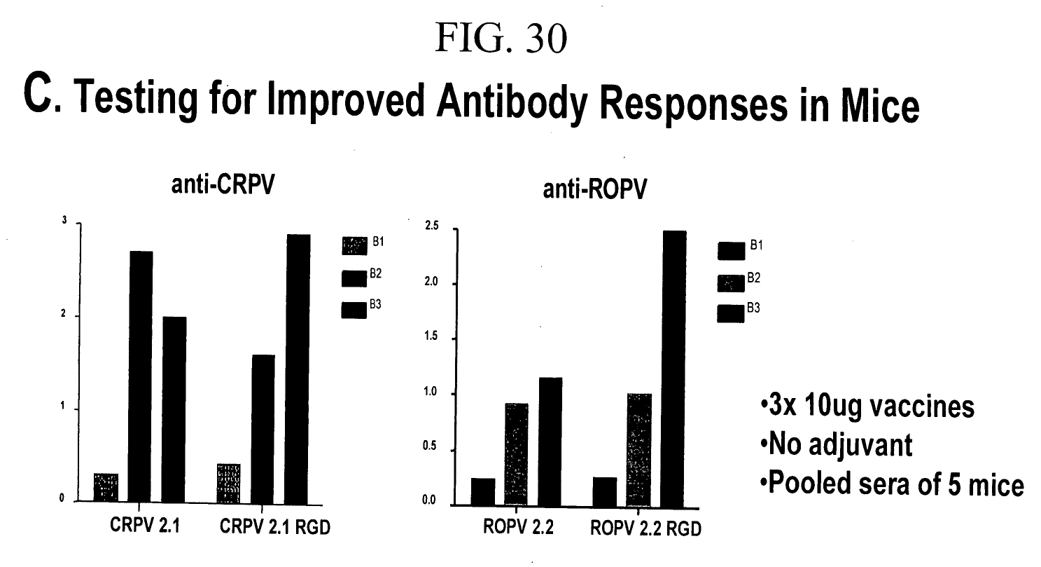

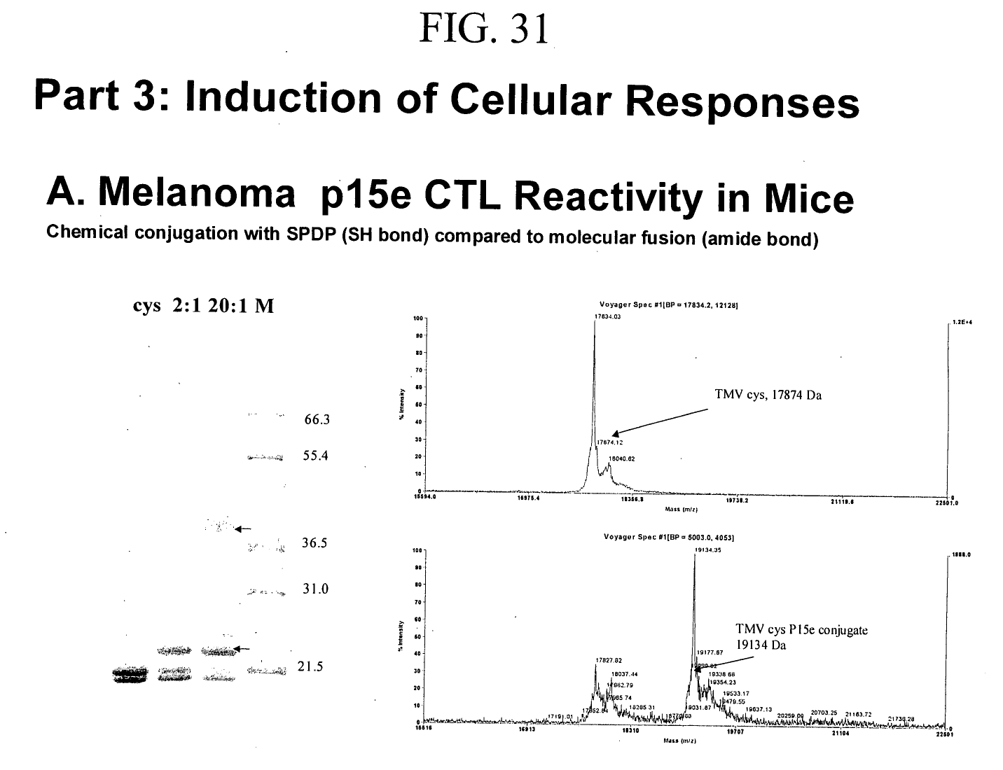

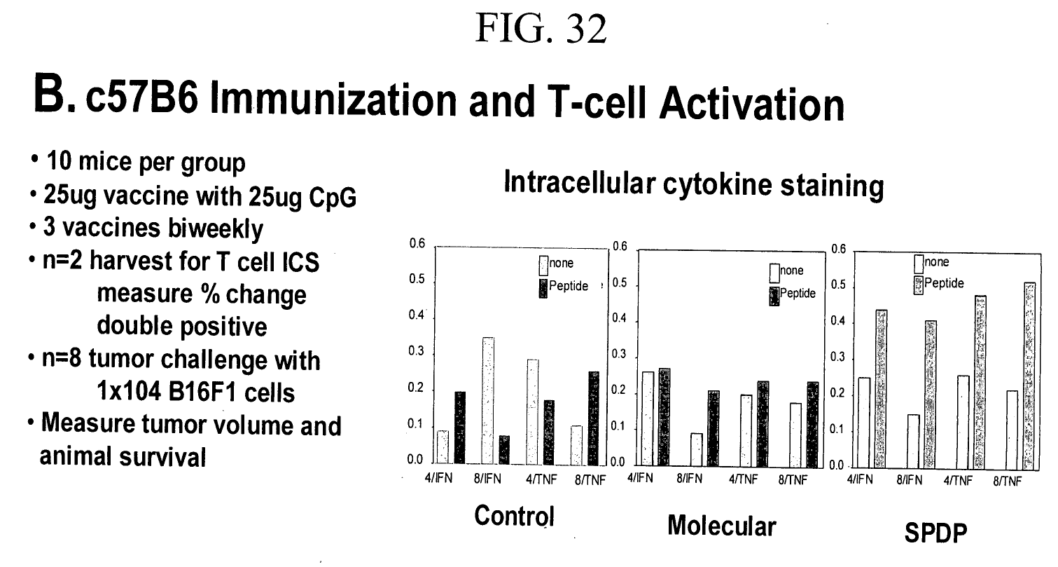

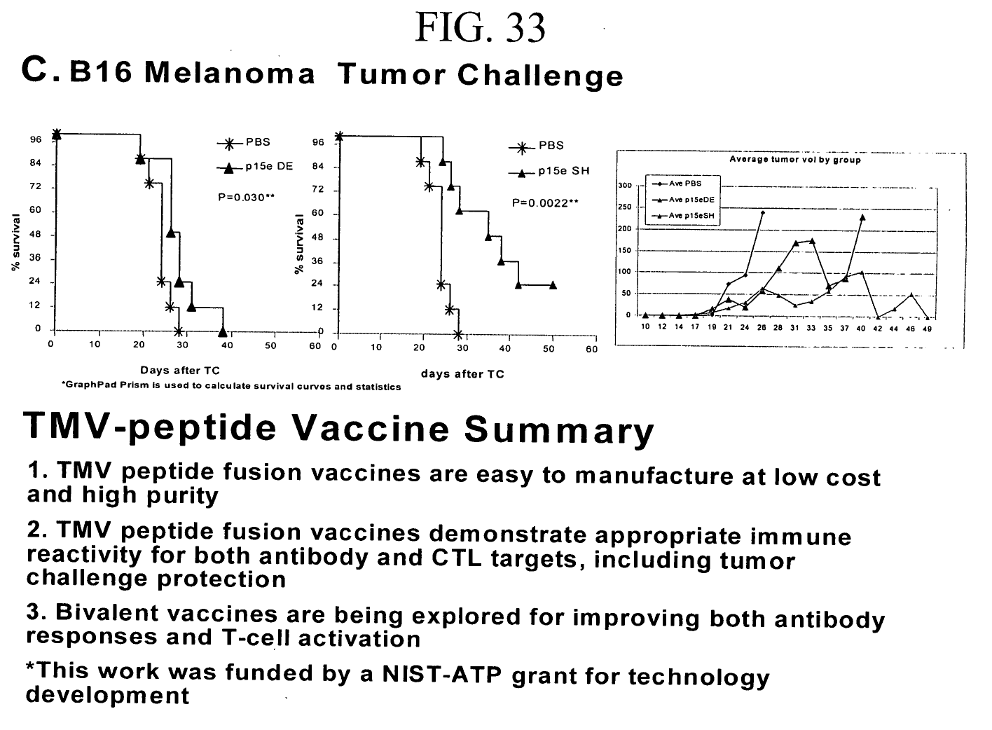

FIGS. 25-33 summarize the use of Tobacco Mosaic Virus epitope display to enhance Papillomavirus L2 antigen presentation, in accordance with the present invention. Goals of the research culminating in the information displayed in FIGS. 25-33 include: tested the Ability of TMV-peptide; fusions to Induce Effective Humoral and Cellular Responses; Built Papillomavirus L2 neutralization epitopes as molecular fusions to TMV coat proteins; >80% fusions up to 17aa are soluble and abundantly expressed in plants; tested for appropriate anti-peptide responses in vaccinated mice; improve immunogenicity using a bivalent (aka dual valent) vaccine strategy; built and testes bivalent fusions; test for augmented immunogenicity; control T cell MHC class I loading: reducible-bond TMV-fusions; p15e self antigen in C57b6 mice with molecular vs SPDP conjugation; Intracellular cytokine T-cell activation; and B16 melanoma tumor challenge.

### EXAMPLE 15

### ELIspot Assay Development for OVA SH

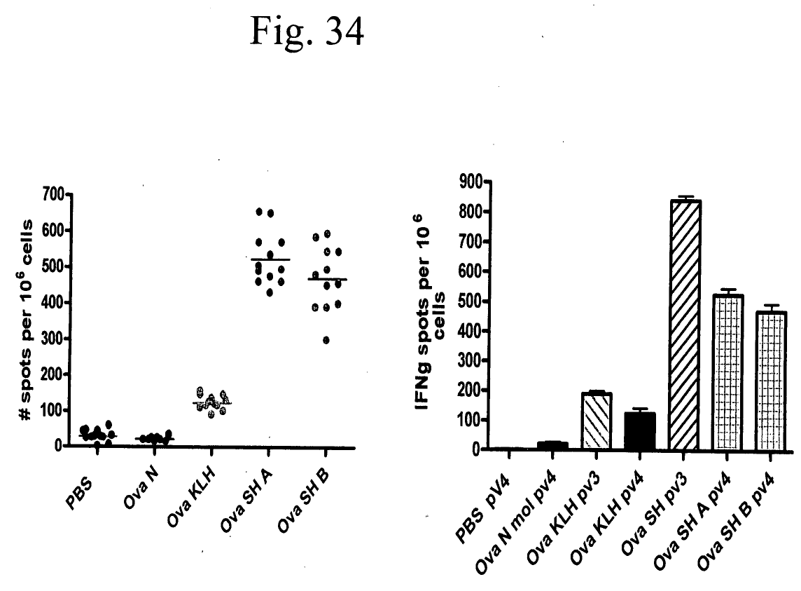

To better understand T cell activation processes relevant to conjugate vaccine immunization, ELISpot Interferon gamma assays on spleen cells from Ova N and Ova SH vaccinated mice were performed. Spleens from immunized mice were removed by sterile excision 5-7 days post vaccination 3 or 4, and processed to single cell suspensions. 2×10⁵ cells were incubated with Ova peptide stimulus for 18 hours on IFNg antibody coated 96 well ELISpot plates. Negative control stimulation with an irrelevant peptide (from bGal;DAPYINTV) generated similar responses to media alone (0-8 spots per 10⁶ cells). Data shown in FIG. 34 indicate that SH conjugate vaccines stimulate high levels of anti-ova T cell responses which are reproducible in two independent animals (Ova SH A and Ova SH B). Shown are multiple replicates of the same cell population (dots), and the median response (bar). Stimulation with an irrelevant peptide generated levels equal to PBS after the third and fourth vaccine. Background levels differed for each assay, but within expected norms. Ova N generated activated T cell responses only after the fourth vaccination. Surprisingly, activated T cell responses are reduced after the fourth Ova SH conjugate vaccination (FIG. 34) and individual animal responses are in close agreement. Ova KLH was used as a positive control vaccine, that also stimulated lower but measurable levels of response after three vaccines, and that level was also reduce upon a fourth immunization. These data confirm that ELISpot method can accurately and reproducibly measure activated T cell responses after vaccination, and that SH conjugate vaccines stimulate a superior level of response as compared to molecular or KLH conjugate vaccines. They also suggest that careful examination of onset and duration of response to SH conjugate vaccine administration is warranted.

### EXAMPLE 16

### Ova Tumor Challenge with SH Monovalent Vaccines

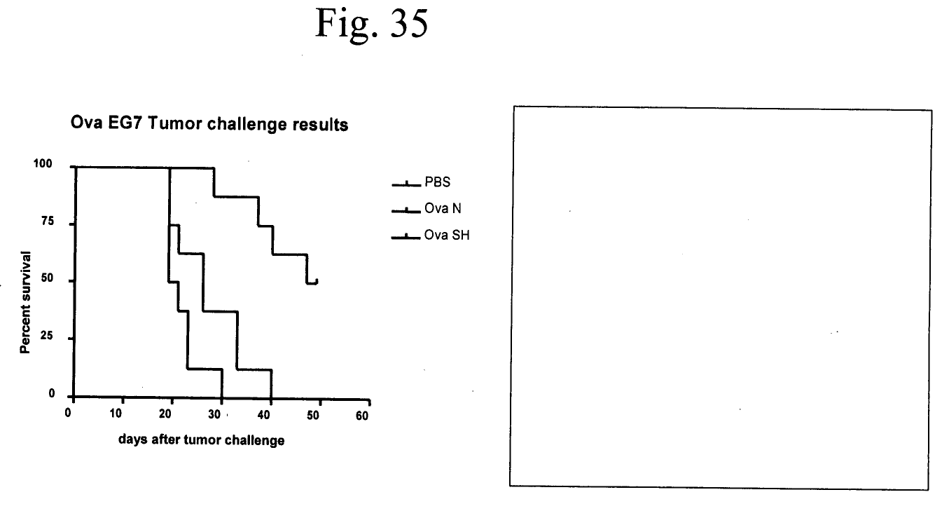

As we have seen in one previous experiment with p15e, SH conjugate vaccines provide superior tumor challenge protection. Ova was used again to show the concept is general and not specific to the antigen or tumor type. After three vaccines of 10 ug each on a biweekly schedule, animals were challenged with a lethal dose of 2×105 OvaEG.7 tumor cells. 10 days following the challenge, animals were monitored and upon tumor onset tumor volume was measured as the longest and widest point. When the 2 dimensional tumor volume reached 2 cm², animals were sacrificed by cervical dislocation or CO2 asphyxiation. Date of death was recorded and Kaplan-Meier survival curves were plotted using GraphPad Prism software. Log-rank statistical analysis of curve comparisons was used to generate “p” values, and significance was set at p=0.05. Shown in FIG. 35, mice given 1×10⁵ tumor cells after PBS control vaccination show and onset of mortality starting at day 18 and 100% mortality by day 30. Animals were immunized three times with the Ova molecular fusion, although providing a 10 day delay in 100% mortality, was not statistically different from the survival of mice given PBS (p=0.055). However, the Ova SH conjugate vaccine with a reducible bond generates superior tumor challenge protection compared to an equivalent dose of Ova molecular vaccine (p=0.0015), and was statistically superior to PBS control mice as well (p=0.00001). These results confirm and support previous findings with p15e, and show that in a second model system, tumor protection is enhanced with a reducible bond TMV fusion.

### EXAMPLE 17

### Time Course for SH Ova Activation

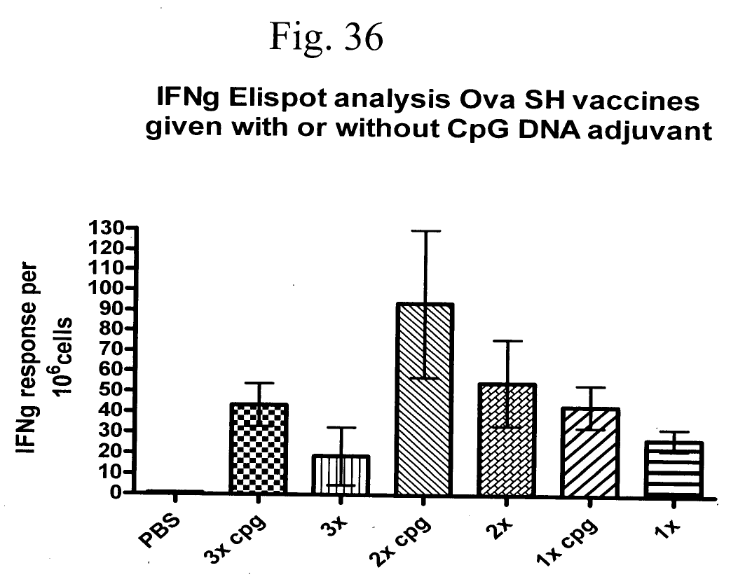

To determine the level of cellular responses to repeated administration of vaccine to induce cellular responses, IFNg Elispot responses after 3, 2 or 1 vaccine, with or without CpG ssDNA adjuvant were measured. Two animals per group were vaccinated with 10 ug Ova SH TMV conjugate vaccine, with or without CpG DNA oligo adjuvant. Two weeks after three, two or a single administration of vaccine, animals were sacrificed, spleens were harvested and 2×10⁵ single cell suspensions were incubated with media, Ova peptide or control peptide overnight on membranes coated with anti-IFNg antibodies. IFNg secretion in response to peptide stimulation was then detected with an HRP conjugated anti-IFNg antibody, and spots counted on an AID spot reader. The data reflects at least six replicates per specific stimulation. Non-specific stimulation was subtracted and numbers were normalized to report IFN activation per 10⁶ cells. The SH chemical conjugate was used, given that the molecular fusion did not show responses until after the fourth immunization, and even then at much lower levels. In FIG. 36, IFNg Elispot results show an overall trend in improved in cellular responses with CpG DNA adjuvant. However, the overall response titers in unadjuvanted vaccine groups followed the same pattern as CpG adjuvanted vaccine groups and with or without adjuvant none of the means varied significantly. Surprisingly, highest titers in each group were found after vaccine 2, and that titer was reduced after vaccine three. It could be possible that fewer activated T cells is a reflection of low affinity T cell anergy, and/or maturation of the response to higher affinity more effective CTL's. Ova tumor challenge after two or three doses should be able to discriminate between the efficacy of larger numbers of or more mature T cells in response to ova conjugate vaccination.

### EXAMPLE 18

### Conjugation Techniques

A SPDP chemical conjugate bond generates superior T cell responses in vaccinated animals compared to a molecular (and not reducible) bond, as measured by both T cell activation in vitro by IFNg Elispot analysis as well as functional T cell responses in tumor challenge models (for both Ova and p15e). One may fuse Ova to TMV by a second chemical linker called SMCC, which uses the same amino acid activation groups, but creates a thiol rather than disulfide bond between the cysteine on the peptide and the sulfhydryl on the linker. Tests show that conjugation efficiencies with the SMCC linker to TMV lysine are approximately equivalent to that obtained with SPDP (˜80%), using the Ova peptide.

### EXAMPLE 19

### Animal Study with Reducible and Non-Reducible Ova Peptide Conjugate and Dissociated SH Conjugate

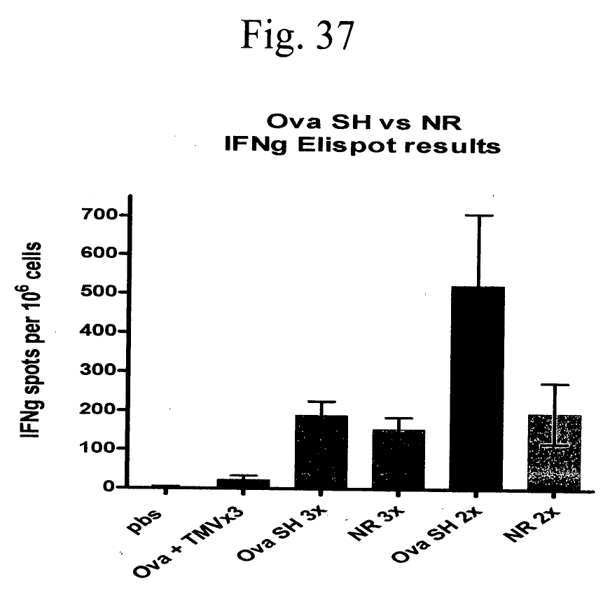

Qualified vaccine material was prepared for SPDP (SH) and SMCC(NR) chemical conjugates, and used in the following vaccine study in C57b/6 mice (FIG. 37). A formulation of TMV Ova that was a mixture of TMV plus an equivalent dose of peptide (Group 2) to use as a vaccine in parallel with conjugate vaccines was prepared.

Study design and IFNg Elispot results for SH vs. NR Ova TMV conjugate vaccines. In FIG. 37, A. Study design to test TMV-SH conjugate made with SPDP, vs. Non-Reducible (NR) TMV conjugate made with SMCC. Ten animals per group were given either 3 (or two doses of 500 ng peptide delivered, associate with between 10-13 ug TMV as either a conjugate or mixed with TMV (Group 2). Eight days after the last vaccine, two mice per group were sacrificed, and single cell spleen suspensions were tested for IFNg secretion after overnight stimulation with Ova peptide. Ova-specific responses were measured by the number of T-cells secreting IFNg, after subtraction of background responses to an irrelevant peptide (in this experiment, an average of 0-5 spots) and normalization to 10⁶ cells. Analysis of mean values indicated that 3× groups were not different, but 2×SH vaccination induced significantly higher levels of activated T cell responses than 2×NR vaccination (p<0.0001).

All animals given TMV peptide conjugates have measurable peptide specific T cells as determined by IFNg secretion. Three doses of TMV mixed with 500 ng peptide gave some response, but significantly lower than other groups, as expected. This demonstrates that linkage to TMV is essential in promoting appropriate cellular immunity. Three doses of TMV Ova conjugates gave statistically similar results, and surprisingly, the TMV Ova vaccine made with the reducible bond also gave similar measures of response. Two doses of Ova SH vaccine gave the highest T-cell activation response levels (as repeated in three independent analyses, and for reasons that are as yet unknown), but the Ova NR fusion stimulated a significantly lower response (p<0.0001).

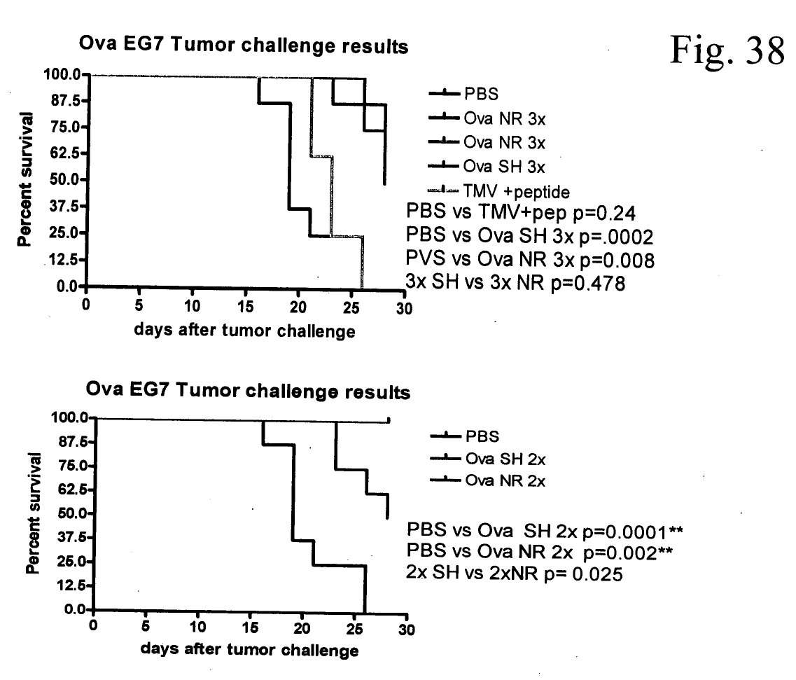

The remaining 8 animals were challenged with 2×10⁵ Ova EG.7 tumor cells, and survival data 28 days post tumor challenge is shown in FIG. 38. It appears that there is a strong correlation between absolute numbers of activated T cells as measured by Elispot and survival. Three doses of TMV mixed with peptide shows a slight delay in the onset of mortality, but is not significantly different than survival of mice given PBS (p=0.24). Mice given three doses of SH or NR, which were not different in T cell activation numbers by Elispot are also not different by tumor challenge curve comparison (p=0.478), and both groups are better than PBS (p<0.01). Two doses of TMV conjugates also reflect Elispot T cell activation analysis, in that both groups have significantly improved protection compared to PBS, and the SH conjugate is significantly better than the NR (p=0.025) and SH 2× was significantly better than 3× (p=0.026).

This data supports the use of TMV vaccines as effective in stimulating T-cell activation, as well as functional T-cell activity against antigen-specific tumor growth. What is surprising is that the type of antigen-TMV linkage has such a great impact on vaccine effectiveness. Reducible disulfide bond linkage appears superior to genetic fusion, and it may be that the linkage is reduced locally after VLP uptake to facilitate release of the peptide at high concentration within the Dendritic cell (DC). An attractive hypothesis is that a specific MHC complex associated thiol reductase enzyme, ERp57 (Hughes, E. A., Cresswell, P., The thiol oxidoreductase ERp57 is a component of the MHC class I peptide-loading complex. Curr Biol, 1998. 8(12): p. 709-712 Dick, T. P., Bangia, N., Peaper, D. R., Cresswell, P., Disulfide bond isomerization and the assembly of MHC class I-peptide complexes. Immunity, 2002. 16(1): p. 87-98.) allows for disulfide reduction and peptide release. The fact that the non-reducible bond fusion has less favorable characteristics seems to support the hypothesis. These data form compelling evidence that the taught new way to target antigen delivery to proteosome-independent pathways of antigen crossover in DC.

### EXAMPLE 20

### Encapsidated SFV RNA

Test SFV Encapsidated RNA for Cellular bGal Expression

As a model replicating RNA, novel Semliki Forest Virus (SFV) expression vectors were used. The SFV genome encodes a 5′ terminal open reading frame translated from the genomic RNA to yield virus replication proteins. The virus structural proteins are produced in animal cells by the action of a subgenomic RNA promoter, similar to that used by TMV in plants, to produce a smaller capped RNA encoding the coat protein and membrane-associated glycoproteins. The bGal gene was inserted in place of the virus structural proteins to generate a non-infectious transcript, and thus bGal expression is a marker for when the SFV vector replicates in a animal cell. A TMV origin of assembly (OAS or ori) was inserted into the SFV-bGal RNA that can then be encapsidated in vitro with wild type U1 TMV coat or a TMV coat fusion. Naked RNA transcripts were transfected into tissue culture cells generating robust bGal expression, suggesting that the addition of the TMV OAS does not interfere with SFV replication.

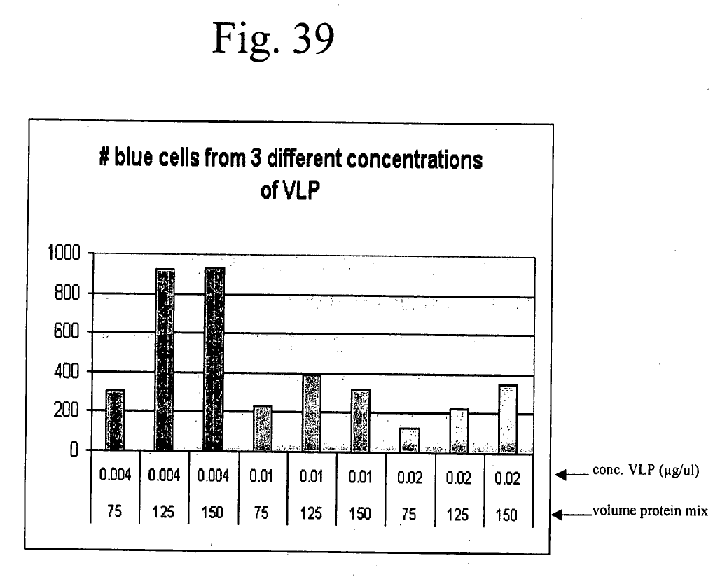

This SFV RNA was then encapsidated in vitro by RNAse free coat preparation of either wild type TMV U1 or U1 RGD, which contains the integrin binding peptide (RGD) known to enhance cellular uptake. Reassembly reactions were qualified, and then tested in uptake experiments into BHK-21 cells, known to express receptors that interact with the RGD motif. (A) BHK-cells were incubated in serum free media with encapsidated SFV-bGal RNA, along with the lipid transfection reagent (Biotrek; Stratagene) at several ratios of VLP to lipid protein mix. After 4 hours, the reaction mixtures were removed and cells were washed and then incubated overnight in complete media. bGal protein was used as a positive control. After 24 hours, cells were formaldehyde fixed, and then stained for bGal reactivity using a substrate that generates a blue precipitate. (B) bGal positive cells were counted as “blue cells”. Hundreds of positive cells were visible in every transfection with TMV coat encapsidated SFV delivered with the lipid reagent, with the best expression levels a the lowest concentration of VLP and the highest level of lipid protein mix (0.004 ug/ul in 150 ul final volume). The data is shown in FIG. 39. Early experiments did not show any spontaneous uptake of TMV, with or without the RGD motif, as indicated by few or no bGal staining cells. Although tissue culture cells are being used as a preliminary test for uptake, they may not contain all of the phagocytic or endocytic mechanisms that characterize mixed populations of primary immune cells in vivo (especially Dendritic, macrophage or B-cells) that would be activated in the presence of a particulate antigen. Therefore the ability of the encapsidated RNA to express bGal after artificial delivery of the TMV-virus like particles into cells was treated using a commercially available lipid protein transfection reagent. The lipid reagent generated robust bGal reporter gene activity in cells incubated with U1 encapsidated SFV RNA. By bypassing uptake requirements, SFV-bGal RNA is uncoated and translated in the cytoplasm of cells. SFV RNA is capable of replication and generates higher levels of protein accumulation, as was evident by increase in the intensity of in blue staining, in comparison to the bGal protein transfection control. Tests to determine immunogenicity of encapsidated SFV-bGal RNA should confirm that bGal expression also occurs in immune cells that take up particulate antigen as described below.

### EXAMPLE 21

### In Vivo B-gal Responses to SFV Vaccines

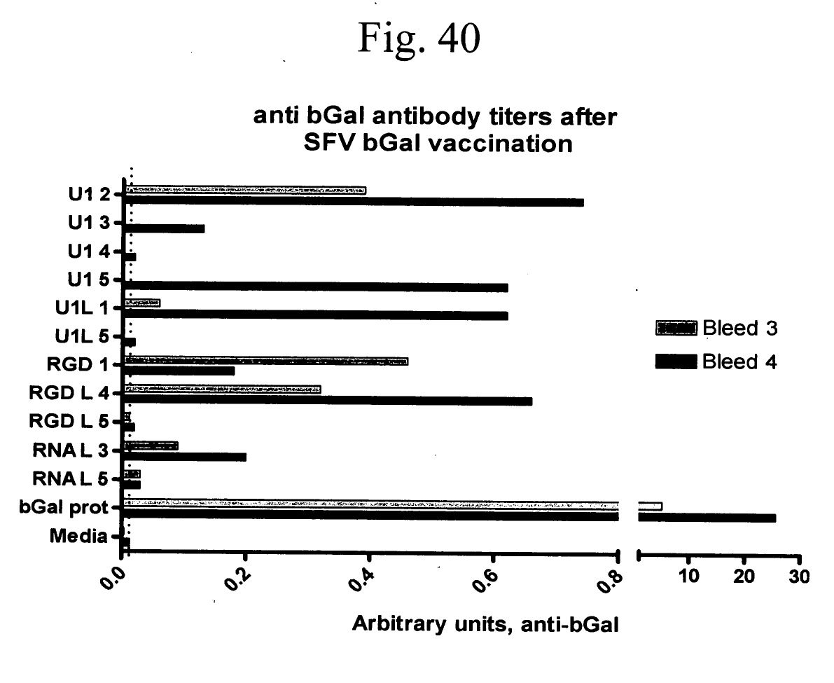

To determine immunogenicity of encapsidated SFV-B-Gal RNA, SFV and control vaccines was produced and qualified to confirm that B-Gal expression also occurs in immune cells that take up particulate antigen. Vaccine compositions included 10 μg RGD TMV coat and U1 TMV coat encapsidated SFV B-Gal RNA. 20 μg of each vaccine type was administered to five animals per group by subcutaneous injection, every 14 days, and sera were collected on day 10 after each vaccine. In some groups, VLP was delivered in lipid at approximately 0.1 μg on the same schedule. Antibody responses to whole protein B-Gal antigen were measured by ELISA. Shown in FIG. 40. Anti-B-Gal responses after SFV B-Gal immunization were shown with responder animals in the SFV vaccine groups compared to RNA plus lipid. Individual animal sera was tested by ELISA for specific reactivity against native B-Gal protein, and an anti-B-Gal monoclonal antibody was used as a positive control as an arbitrary standard. Average responses are shown for positive control vaccine B-Gal protein, and for negative control vaccine media. Results were, measured 10 days after the fourth vaccine, although responses were detected after the second immunization in most groups.

As shown in FIG. 40, individual animal responses to TMV SFV-B-Gal vaccine groups were varied, with about half the animals responding at higher levels and half responding at detectable levels and just above the minimum cut off of two fold over background. In U1 encapsidated SFV vaccine groups, four out of five animals demonstrated specific anti-B-Gal antibody responses after four immunizations, with two animal responding at low levels and two animals responding at high levels, one of which was boosted strongly after the second and the third immunizations (mouse U1 2). Although the VLP was administered at a much lower dose, addition of lipid also stimulated appropriate anti-B-Gal responses in 2 of 5 animals. One animal responded to RGD encapsidated SFV vaccination, and that was improved slightly with lipid co-administration with two responding animals per group. RNA plus lipid vaccine groups showed low but measurable responses in three out of five mice, while immunization with naked RNA or CMV promoter driven DNA did not generate antibody titer responses above background (data not shown).

Confirming early in vitro data, these results clearly demonstrate the ability of TMV encapsidated SFV-B-Gal RNA to uncoat and become accessible for translation. Translation products are then presented to the immune system, resulting in accumulation of anti-B-Gal antibodies. These examples show that a TMV encapsidated RNA can deliver a functional translatable nucleic acid to recipient cells.

### EXAMPLE 22

### SFV Encapsidated RNA Can Stimulate Appropriate Cellular Immunity

Encapsidated RNA may be used in a therapeutic application to stimulate cytotoxic T cell activation from an MHC class I restricted epitope from an internally expressed protein product. A fifth immunization was administered for a select set of animals, including one mouse each from control groups (media, B-Gal DNA, or B-Gal protein) and one mouse each from experimental U1 SFV or RGD SFV vaccine groups. Five days following the immunization, spleens were excised and single cell suspensions were stimulated with the the second C57b/6 restricted CTL peptide derived from B-Gal (ICPMYARV; Oukka et al. k, 1996; J. Immunol 156:968), or whole B-Gal protein stimulation, and IFNg secretion was measured by ELISpot as compared to unstimulated controls. As shown in FIG. 8, all groups immunized with B-Gal showed robust IFNg secretion when stimulated with either peptide or whole B-Gal protein. Protein immunized mice showed the highest response to whole protein immunization, while all other B-Gal immunization groups were approximately equivalent except for U1, which responded less well to protein stimulus than peptide.

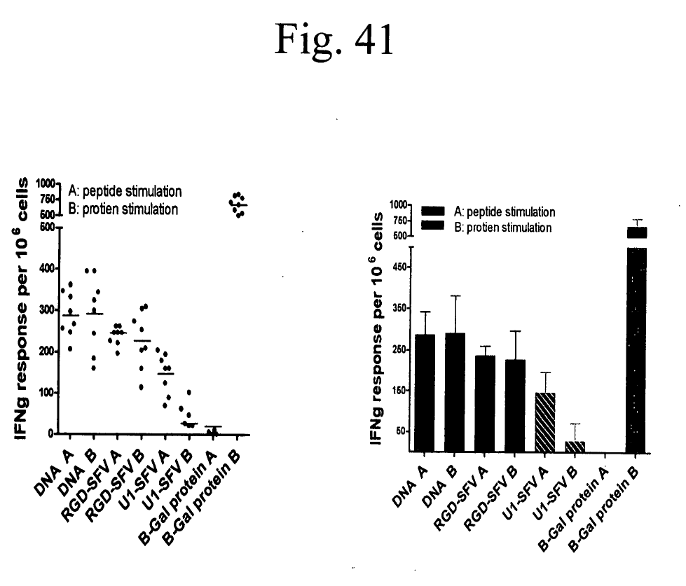

Five days after the last immunization, spleens from immunized mice were removed by sterile excision and processed to single cell suspensions. 2×10⁵ cells were incubated with 1 μM B-Gal peptide (A) or 50 μM B-Gal protein (B) stimulus for 22 hours on IFNg antibody coated 96 well ELISpot plates. Average response to media alone were subtracted from each group. The data is shown in FIG. 41.

Peptide stimulated cells showed strikingly different responses, with nucleic acid immunization groups demonstrating high response levels, and protein immunization demonstrating no response, indicating that the IFNg secretion response to protein stimulation is most likely non-T cell mediated. Even though antibody responses to B-Gal were at background levels for nucleic acid immunization groups, T-cell activation was still present. This suggests that intracellular expression of the B-Gal protein is stimulating appropriate MHC class I peptide loading, and that T cells that are capable of binding peptide are present prior to stimulation, but can also respond to whole antigen.

### EXAMPLE 23

### SFV bGal Prime Boost Results, ELISA and IFNg Elispot

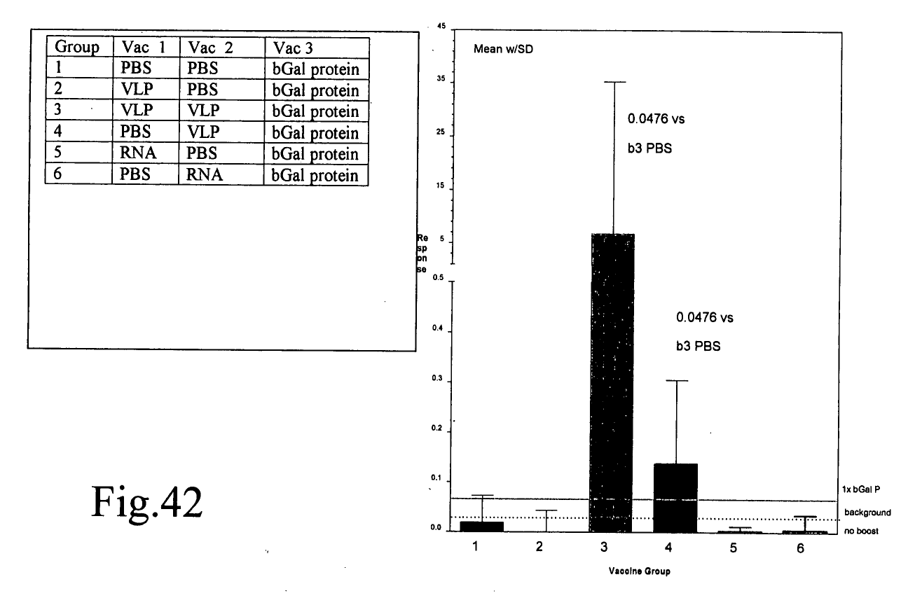

The first test of encapsidated SFV RNA efficacy demonstrated that SFV RNA can release TMV upon cell uptake, localize to the cytoplasm of cells, and be translated with cellular ribosomes into functional protein. Using the marker translation product beta Galactosidase (bGal), bGal activity was observed in cells in vitro and anti-bGal antibodies and cellular responses after vaccination in vivo as demonstrated and described above. A prime boost strategy that examined the ability of TMV-encapsidated SFV bGal RNA to effectively prime a single subsequent protein boost. Without knowing the kinetics of priming, three schedules were tested; a single TMV-SFVbGal immunization four weeks prior to protein boosting (Group 2; or VPb), a dual TMV-SFVbGal vaccine four and two weeks prior to protein boosting (Group 3; or VVb), and a single TMV-SFVbGal prime two weeks prior to protein boosting (Group 4; or pVb). Naked RNA priming was also tested as a control (Groups 5 and 6), and all groups were compared to bGal protein given once with no priming (Group1). See the data in FIG. 42.

Group descriptions and ELISA results from an SFV prime bGal protein boost study. To study the effect of SFV priming followed by protein boosting, the following study was designed (A). Five animals per group were given 20 ug TMV encapsidated lacZ encoding SFV RNA (VLP) by s.c. injection, or PBS control, or the molar equivalent of naked RNA (1 ug). A second immunization was given, either with encapsidated SFV VLP, RNA or PBS as shown. The last immunization was a single dose of 25 ug bGal protein (from Sigma). As shown in B, are the anti-bGal ELISA results obtained from sera collected 10 days post vaccine 3. Responses are measured as arbitrary units, as compared to a known amount of Goat anti-bGal anti-sera. The background with no boost (post vaccine 2) is shown as a dotted line, and the anti-bGal response after a single protein immunization are shown as a solid grey line (1xbGalP).

Antibody responses were tested by ELISA analysis against bGal protein using sera collected 10 days after vaccine 2 (data not shown; responses at background levels) and after the protein boost (FIG. 11B). Groups given VLP twice (Group 3), or VLP once two weeks prior to protein boost (Group 4) responded with significant antibody titers compared to PBS. Although mean responses vary by significant amounts between groups 3 and 4, p value as calculated by an unpaired t-test (Mann-Whitney) show they are not significantly different p=0.06). VLP given four weeks prior (Group 2), or given naked RNA priming failed to induce measurable responses over that of a single dose of bGal protein alone (11B, grey line). Of note, two VLP priming doses followed by a single bGal protein boost gave mean titers at 6 units, as compared to average values of 0.4 or 0.6 after 3 or 4 doses of VLP alone without a protein boost (FIG. 42), with three of five animals responding at 7, 10 or 66 units, and two animals responding at 0.2 or less. These results demonstrate that VLP vaccines can prime effectively.

Cellular responses were also measured in two mice per group at day 10 after the protein boost. Antibody responses suggest favorable boosting with SFV bGal priming two times, and cellular responses analyzed in two mice per group indicated that two doses of VLP prior to protein boost responses (Group 3 VVb; VLP/VLP/bGal) were significantly better (p<0.0001) than a single dose of VLP either four (Group 2 VPb; VLP/PBS/bGal) or two weeks prior to protein boosting (Group 4; PVb). No response was measured with a single protein boost.

### EXAMPLE 24

### Stimulation of IFNg Secretion by bGal Peptide after SFVbGal VLP Prime Boost

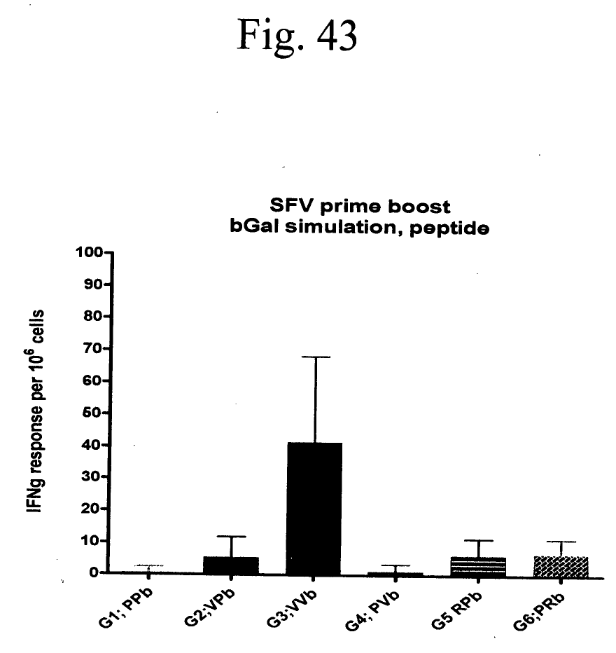

Spleens from two mice were made into single cell suspensions, and then stimulated overnight with bGal peptide. IFNg secretion was detected by IFNg Elispot, and responses were normalized to 10⁶ cells. Background stimulation with an irrelevant peptide was essentially zero for all groups. See the data in FIG. 43.

1. A method of making a virus-like particle (VLP) containing a nucleic acid encoding a gene that is capable of expression in a eukaryotic cell, the method comprising the steps of:
a) disassembling virus to coat protein or encapsulation intermediate;
b) optionally forming one or more groups of encapsidation intermediate populations;
c) mixing portions of one or more groups of coat protein and/or encapsidation intermediates;
d) forming intact VLP of one or more coat proteins or encapsidation intermediates surrounding nucleic acid which encodes a gene such that the arrangement of sequences allows translation and gene expression in an animal cell, and stabilization of the nucleic acid for delivery to animal cells or tissues by a VLP structure.

2. A method for making a virus-like particle (VLP) containing multiple, different in composition, peptides or proteins, said method comprising the steps of:
a) disassembling separate virus or VLP populations, each displaying a distinct peptide or protein via genetic fusion;
b) forming coat protein or encapsidation intermediate populations each displaying a distinct peptide or protein;
c) mixing coat proteins or encapsidation intermediates from different populations;
d) forming intact VLP surrounding a nucleic acid core that is composed of different coat proteins or encapsidation intermediates such that the VLP displays more than one peptide or protein.

3. A method as set forth in claim 2, wherein:
said VLP is a tobacco mosaic virus (TMV) virus-like particle (VLP) containing multiple, different composition peptides or proteins;
in said disassembling step the separate virus or VLP populations are TMV populations; and
in said first forming step, said mixing, and said second forming step, the encapsidation intermediate populations of 20S disks are used.

4. A method for making a virus-like particle (VLP) containing multiple, different composition peptides, proteins or nucleic acid moieties, said method comprising the steps of:
a) disassembling a virus or
VLP population that has a surface residue for chemical conjugation, provided by genetic fusion;
b) forming coat proteins or encapsidation intermediates;
c) effecting chemical conjugation of unique peptide, protein, nucleic acid and/or other moieties to each of several separate coat protein or encapsidation intermediate populations;
d) mixing coat proteins and/or encapsidation intermediates from different populations in the presence of a nucleic acid scaffold;
e) forming intact VLP surrounding a nucleic acid core that is composed of different encapsidation intermediates such that the VLP displays more than one moiety, such as a peptide, protein, nucleic acid, other moiety or combination thereof.

5. A method for making a virus containing one or more, different composition peptides, proteins, nucleic acids and/or other moieties displayed, said method including the steps of:
a) constructing an expression vector with
i) a gene for expression in animal cells placed downstream, 3′, of an internal ribosome initiation sequence (IRES) that lies either within the gene or separately placed downstream, 3′, of the gene;
ii) a coat protein expressed from a non-native subgenomic promoter downstream of the gene for expression in animal cells;
iii) the coat protein having either a genetic fusion for the expression of a peptide sequence, protein and/or a surface residue for chemical conjugation, provided by genetic fusion;
b) purifying the viruses expressing peptide, protein and/or surface residue;
c) optionally effecting chemical conjugation of unique peptide, protein, nucleic acid and/or other moieties to purified virus containing a surface residue for chemical conjugation;
d) using virus with genetic fusion and/or chemical conjugation peptides, proteins, nucleic acids and/or other moieties for the stabilization and delivery of the RNA expression construct into animal cells or tissues.

6. A method as set forth in claim 4, wherein:
said VLP is a tobacco mosaic virus (TMV) virus-like particle (VLP) containing multiple, different composition peptides or proteins;
in said disassembling step the separate virus or VLP populations are TMV populations; and
in said first forming step, said mixing, and said second forming step, the encapsidation intermediate populations of 20S disks are used.

7. A method as set forth in claim 4, wherein in said forming step, the VLP displays at least three peptides, proteins, nucleic acids and/or other moieties.

8. A method for making a virus-like particle (VLP) containing multiple, different composition peptides, proteins, nucleic acids and/or other moieties displayed by a process comprising the steps of:
a) disassembling separate VLP populations, each displaying a distinct peptide or protein via genetic fusion;
b) disassembling a separate VLP population that has a surface residue for chemical conjugation, provided by genetic fusion;
c) optionally forming encapsidation intermediate populations such that:
i) each displays a distinct peptide or protein and
ii) each displays a surface residue for chemical conjugation;
d) effecting chemical conjugation of unique peptide, protein, nucleic acid and/or other moieties to separate populations of coat proteins or encapsidation intermediates displaying surface residue for chemical conjugation;
e) mixing the coat proteins or encapsidation intermediates from different populations displaying peptides or proteins by genetic fusion or displaying peptides, proteins, nucleic acids and/or other moieties by chemical conjugation;
f) forming an intact VLP surrounding a nucleic acid core that is composed of different coat proteins or encapsidation intermediates such that the VLP displays more than one moiety, be it peptide, protein, nucleic acid, other moiety or some combination of these moieties.

9. A method as set forth in claim 8, wherein the VLPs are TMV virus and the encapsidation intermediates are 20S disks.

10. A VLP produced by any one of the methods recited in claims 1-9, wherein multiple different peptides, proteins, nucleic acid and/or other moieties are displayed on said VLP such that said VLP induces an immune response against two or more organisms.

11. A VLP produced by any one of the methods recited in claims 1-9, wherein multiple different peptides, proteins, nucleic acids and/or other moieties are displayed on said VLP such that said VLP induces in a host an immune response to one or more epitopes.

12. A VLP produced by any one of the methods recited in claims 1-9, wherein multiple different peptides, proteins, nucleic acids and/or other moieties are displayed on said VLP such that said VLP exhibits an enhanced cellular uptake in the host.

13. A VLP produced by any one of the methods recited in claims 1-9, wherein multiple different peptides, proteins, nucleic acids and/or other moieties are displayed on said VLP such that said VLP exhibits immune stimulation or modulation functions thereof in a host.

14. A VLP made by the methodology as set forth in any one of claims 1-9, said VLP including a nucleic acid moiety that contains a gene for one or more of the following functions: induction of humoral immune responses, induction of cellular immune responses, or stimulation or modulation of host immune responses.

15. A VLP made by the methodology as set forth in any one of claims 1-9, said VLP including a nucleic acid moiety that contains a gene for one or more of the following: an intact or partial viral antigen, an intact or partial bacterial antigen, an intact or partial mycoplasm antigen, an intact or partial eukaryotic pathogen antigen, a cytokine, a chemokine, and a portion of a chemokine, cytokine or cellular receptor that could modulate host immune response.

16. A VLP made by the methodologies as set forth in any one of the claims 1-9, that by virtue of the moieties displayed on the surface and/or the functionality associated with the nucleic acid core, has the properties required to function as a vaccine, an anti-allergy medication, a diagnostic reagent or a combinatorial chemistry reagent.

17. A VLP made by any one of the methods set forth in claim 1, comprising:
an RNA moiety comprising any one from the following group:
an expression vector containing a gene for inducing or modulating host immune responses via expression in mammalian cells;
an expression vector containing an internal ribosome initiation sequence (IRES) upstream of a gene for inducing or modulating host immune responses via expression in mammalian cells;
an origin of assembly (OAS) and a gene for inducing or modulating host immune responses via expression in animal cells;
an Omega RNA leader, origin of assembly (OAS) and a gene for inducing or modulating host immune responses via expression in mammalian cells;
an alphavirus replicon an origin of assembly (OAS) and a gene for inducing or modulating host immune responses via expression gene in mammalian cells;
a rubivirus replicon an origin of assembly (OAS) and a gene for inducing or modulating host immune responses via expression gene in mammalian cells;
a nodavirus replicon containing an origin of assembly (OAS) and a gene for inducing or modulating host immune responses via expression gene in mammalian cells; and
a flavivirus replicon containing an origin of assembly (OAS) and a gene for inducing or modulating host immune responses via expression gene in mammalian cells.

18. A viral coat protein comprising a surface presented, unpaired cysteine residue on the surface of a virus coat protein, constructed by genetic expression of a unpaired cysteine residue at the N, C and/or a surface exposed loop of the coat protein, to augment specific chemical conjugation reactions.

19. A viral coat protein comprising a surface presented, lysine residue on the surface of the virus coat protein, constructed by genetic expression of a lysine residue at the N, C and/or a surface exposed loop of the coat protein, to augment specific chemical conjugation reactions.

20. A viral coat protein fusion, comprising:
a peptide or protein of interest genetically fused to a viral coat protein and flanked by additional charged amino acids introduced to improve viral coat protein fusion solubility, accumulation and/or extraction from an infected plant host.

21. A virus or VLP comprising the viral coat protein fusion as set forth in claim 20.

22. A VLP comprising a plurality of the viral coat proteins of claim 18.

23. A VLP comprising a plurality of the viral coat proteins of claim 19.

24. An encapsidation intermediate comprising a plurality of different viral coat proteins of claim 18.

25. An encapsidation intermediate comprising a plurality of different viral coat proteins of claim 19.

26. An encapsidation intermediate comprising a plurality of viral coat proteins produced by any one of the methods recited in claims 1-9, wherein multiple different peptides, proteins, nucleic acids and/or other moieties are displayed on said encapsidation intermediate such that said encapsidation intermediate induces in a host an immune response to two or more epitopes.

27. A viral coat protein having at least two different peptides, proteins, nucleic acids and/or other moieties epitopes are displayed on the viral coat protein surface at different locations on the viral coat protein molecule, such that the viral coat protein induces in a host an immune response to at least two of the epitopes.

28. A method for making a virus containing two or more, different composition peptides, proteins, nucleic acids and/or other moieties displayed on a viral coat protein, said method comprising;
synthesizing a viral nucleic acid having two copies of a viral coat protein gene, wherein each gene copy contains a different epitope of a peptide, protein, or a surface residue for chemical conjugation, provided by genetic fusion and encoded therein,
replicating the virus in a host cell,
optionally, effecting chemical conjugation of unique peptide, protein, nucleic acid and/or other moieties to the surface residue for chemical conjugation and,
recovering the virus.

29. A virus containing two or more, different composition peptides, proteins, nucleic acids and/or other moieties displayed on two or more viral coat proteins produced by the method of claim 28.


SEQUENCE LISTING (22 sequences)

SEQ ID NO: 1 - protein, 15 aa, Simian Virus 5
GKPIPNPLLGLDSTK

SEQ ID NO: 2 - protein, 10 aa, Influenza virus
YPYDVPDYAK

SEQ ID NO: 3 - protein, 11 aa, human antibody
EQKLISEEDLK

SEQ ID NO: 4 - protein, 19 aa, cottontail rabit papillomavirus
VGPLDIVPEVADPGGPTLV

SEQ ID NO: 5 - protein, 18 aa, cottontail rabit papillomavirus
PGGPTLVSLHELPAETPY

SEQ ID NO: 6 - protein, 19 aa, rabbit oral papillomavirus
VGPLEVIPEAVDPAGSSIV

SEQ ID NO: 7 - protein, 18 aa, rabbit oral papillomavirus
PAGSSIVPLEEYPAEIPT

SEQ ID NO: 8 - protein, 9 aa, Human papillomavirus
AALQAIELM

SEQ ID NO: 9 - protein, 9 aa, mouse tyrosinase
SVYDFFVWL

SEQ ID NO: 10 - protein, 8 aa, mouse
KSPWFTTL

SEQ ID NO: 11 - protein, 8 aa, chicken
SIINFEKL

SEQ ID NO: 12 - protein, 7 aa, Human immunodeficiency virus
ELDKWAS

SEQ ID NO: 13 - protein, 16 aa, respiratory syncytial virus
CEYNVFHNKTFELPRA

SEQ ID NO: 14 - protein, 17 aa, tetanus
QYIKANSKFIGITELKK

SEQ ID NO: 15 - protein, 9 aa, Human IL1beta
VQGEESNDK

SEQ ID NO: 16 - protein, 19 aa, Measles virus
FAGVVLAGAALGVATAAQI

SEQ ID NO: 17 - protein, 7 aa, human integrin
SGRGDSG

SEQ ID NO: 18 - protein, 6 aa, human laminin
GYIGSR

SEQ ID NO: 19 - DNA, 489 bp, Tobacco mosaic virus
atgggatgtggatcttacagtatcactactccatctcagttcgtgttcttgtcatcagcgtgggccgacccaatagagttaattaatttatgtactaatgccttaggaaatcagtttcaaacacaacaagctcgaactgtcgttcaaagacaattcagtgaggtgtggaaaccttcaccacaagtaactgttaggttccctgacagtgactttaaggtgtacaggtacaatgcggtattagacccgctagtcacagcactgttaggtgcattcgacactagaaatagaataatagaagttgaaaatcaggcgaaccccacgactgccgaaacgttagatgctactcgtagagtagacgacgcaacggtggccataaggagcgcgataaataatttaatagtagaattgatcagaggaaccggatcttataatcggagctctttcgagagctcttctggtttggtttggacctctggtcctgcaacttga

SEQ ID NO: 20 - DNA, 489 bp, Tobacco mosaic virus
atgggaaaaggatcttacagtatcactactccatctcagttcgtgttcttgtcatcagcgtgggccgacccaatagagttaattaatttatgtactaatgccttaggaaatcagtttcaaacacaacaagctcgaactgtcgttcaaagacaattcagtgaggtgtggaaaccttcaccacaagtaactgttaggttccctgacagtgactttaaggtgtacaggtacaatgcggtattagacccgctagtcacagcactgttaggtgcattcgacactagaaatagaataatagaagttgaaaatcaggcgaaccccacgactgccgaaacgttagatgctactcgtagagtagacgacgcaacggtggccataaggagcgcgataaataatttaatagtagaattgatcagaggaaccggatcttataatcggagctctttcgagagctcttctggtttggtttggacctctggtcctgcaacttga

SEQ ID NO: 21 - protein, 15 aa, Artificial Sequence
  Created from Seq ID No. 10 inserted into TMV
AMKSPWFTTLAGPAT

SEQ ID NO: 22 - protein, 17 aa, Artificial Sequence
  Created from Seq ID No 10 inserted into TMV
AMDEKSPWFTTLAGPAT